(19) United States
(12) Patent Application Publication
(10) Pub. No.: US 2012/0088262 A1
(43) Pub. Date: Apr. 12, 2012

(54) CYANINE COMPOUNDS, CONJUGATES AND METHOD OF USE

(75) Inventors: Ali Dehghani, Campbell, CA (US); Kamala Tyagarajan, Fremont, CA (US); Kimvan Tran, Hayward, CA (US)

(73) Assignee: Millipore Corporation, Billerica, MA (US)

(21) Appl. No.: 13/267,127

(22) Filed: Oct. 6, 2011

Related U.S. Application Data
(60) Provisional application No. 61/408,519, filed on Oct. 29, 2010.
(60) Provisional application No. 61/390,606, filed on Oct. 6, 2010.

(51) Int. Cl. C12Q 1/02; C07D 209/08; C07D 209/58; C07H 19/00; C07K 2/00; C07K 14/00; C07H 99/00; C07K 16/00; C07D 263/56; C12Q 1/00

(52) U.S. Cl. 435/29; 548/217; 548/455; 548/427; 435/40.5; 530/300; 530/350; 536/123.1; 530/391.3; 536/22.1; 530/359; 530/396

(57) ABSTRACT

Cyanine compounds having the general formula I, conjugates, complexes, and compositions comprising the cyanine compounds are provided. Fluorescence resonance energy transfer (FRET) dye pairs and viability dyes are also provided.

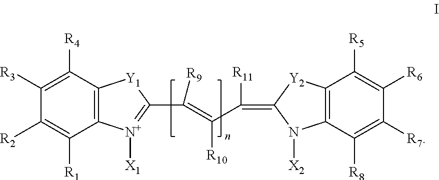

### CROSS REFERENCES TO RELATED APPLICATIONS

This application claims the priority to and benefit of U.S. Provisional Application No. 61/390,606, filed Oct. 6, 2010 and entitled “Fluorescence Resonance Energy Transfer Dye Pairs, Viability Dyes, and Method of Use,” and U.S. Provisional Application No. 61/408,519, filed Oct. 29, 2010 and entitled “Cyanine Compounds, Conjugates and Method of Use,” the disclosures of all of which are incorporated herein by reference in its entirety.

### BACKGROUND

This invention relates in general to cyanine compounds and in particular to optically active cyanine compounds, conjugates comprising such compounds, methods of making and using them.

Cyanine compounds have been widely used in industries e.g. in photography, textile dyeing, and in CD-R and DVD-R media. Cyanine compounds also find use as fluorescent labels in bioassays, either as single labels or in energy transfer schemes employing multiple labels. The explosion of bioinformatics, array technology, and genome projects over the last decade has led to a great need for environmentally acceptable labels such as fluorophores to provide information on the physical sequence of biomolecules, on the expression of genes at the polynucleotide and protein level, and on the actual location of biomolecules in cells, tissues and organisms. Fluorescent labels can also be used to detect cell-specific markers and characterize and separate specific cell populations and subpopulations using cytometric methods. These techniques typically use a fluorescent molecule such as a cyanine conjugated to a biomolecule such as an antibody or a nucleotide or dye terminator.

Fluorescence resonance energy transfer (FRET) schemes have frequently been used in bioassays. FRET assays rely on measuring the rate of non-radioactive transfer from the excited state of one fluorophore (donor) to another fluorophore (acceptor), which may emit detectable energy or transfer it to a subsequent species. The key to any FRET assays is the selection of dye pairs as acceptor and donor fluorophores. A dye pair when brought in molecular proximity must possess sufficient spectral overlap of the emission spectrum of the donor and the excitation spectrum of the acceptor so that they can cause re-emission in their own characteristic wavelengths.

A continuing need in this field is the development of FRET dye pairs. There is a need for FRET dye pairs that can be excited by the sources that are commonly found in flow cytometry or other imaging systems. There is a need for FRET dye pairs whose emission can be detected in the detection windows commonly found in these instruments.

Assessment of the percentage of live cells in a sample is important in flow cytometry to accurately interpret test results. Dead cells or cells with damaged membranes may nonspecifically bind to probes, causing misinterpretations of test results.

Conventional DNA intercalating viability or dead cell dyes such as propidium iodide (PI) and 7-AAD etc. cannot be used with wash or fixation and permeabilization conditions. Their usage is further diminished due to limited spectral windows. Amine reactive viability dyes have advantages in the evaluation of cell's viability when assay conditions require washing or treatment with fixation and permeabilization reagents. They are based on the principle that an intact cell has fewer exposed proteins thus fewer amino groups on the cell surface. When the cell membrane is compromised or damaged, a larger number of inward-facing or intracellular amino groups are exposed and these cells depict a high level of staining with amine reactive fluorescent dyes.

A continuing need is the development of viability or dead cell dyes which can be used in combination with intracellular or extracellular markers where washing or fixation and permeabilization conditions may be required. The availability of viability dyes in a variety of excitation and emission spectra would provide the flexibility when designing staining panels for multicolor flow cytometry to provide more comprehensive and accurate identifications of appropriate cell populations.

### SUMMARY

Cyanine compounds, compositions containing them, and methods of making and using them are provided. The cyanine compounds comprise one or more carboxyl groups or derivatives thereof that are indirectly attached to an aryl ring on an alkylaryl cyanine substituent. Also provided are conjugates of the disclosed cyanine compounds and one or more other substances. Complexes comprising the disclosed cyanine compounds, and compositions and articles comprising the cyanines are also provided. Fluorescent dye pairs useful in FRET assays are provided. Cyanine based amine reactive viability dyes are also provided. Other embodiments are described further herein.

### BRIEF DESCRIPTION OF THE DRAWINGS

These and various other features and advantages will become better understood upon reading of the following detailed description in conjunction with the accompanying drawings and the appended claims provided below, where:

FIG. 1 is a fluorescence excitation and emission spectrum for Compound No. 4 (Ex Peak 560 nm, Em 579 nm) in accordance with one embodiment;

FIG. 2 is a fluorescence excitation and emission spectrum for Compound No. 5 (Ex Peak 560 nm, Em 580 nm) in accordance with another embodiment;

FIG. 3 is a fluorescence excitation and emission spectrum for Compound No. 6 (Ex Peak 650 nm, Em 682 nm) in accordance with another embodiment;

FIG. 4 is a fluorescence excitation and emission spectrum for Compound No. 7 (Ex Peak 650 nm, Em 682 nm) in accordance with another embodiment;

FIG. 5 is a fluorescence excitation and emission spectrum for Compound No. 9 (Ex Peak 489 nm, Em 507 nm) in accordance with another embodiment;

FIG. 6 is a fluorescence excitation and emission spectrum for Compound No. 10 (Ex Peak 489 nm, Em 507 nm) in accordance with another embodiment;

FIG. 7 is a fluorescence excitation and emission spectrum for Compound No. 11 (Ex Peak 510 nm, Em 542 nm) in accordance with another embodiment;

FIG. 8 is a fluorescence excitation and emission spectrum for Compound No. 12 (Ex Peak 510 nm, Em 541 nm) in accordance with another embodiment;

FIG. 9 is a fluorescence excitation and emission spectrum for Compound No. 13 (Ex Peak 609 nm, Em 641 nm) in accordance with another embodiment;

FIG. 10 is a fluorescence excitation and emission spectrum for Compound No. 14 (Ex Peak 609 nm, Em 642 nm) in accordance with another embodiment;

FIG. 11 a is fluorescence excitation and emission spectrum for Compound No. 16 (Ex Peak 609 nm, Em 642 nm) in accordance with another embodiment;

FIG. 12 is a fluorescence excitation and emission spectrum for Compound No. 18 (Ex Peak 551 nm, Em 574 nm) in accordance with another embodiment;

FIG. 13 is a fluorescence excitation and emission spectrum for Compound No. 19 (Ex Peak 650 nm, Em 674 nm) in accordance with another embodiment;

FIG. 14 shows the impact of different Dye to Protein ratios on the fluorescence of M1 antibody conjugates in accordance with some embodiments;

FIG. 15 shows the utility of M1 conjugates in cellular applications in accordance with some embodiments;

FIG. 16 shows the photobleaching characteristics of M1 antibody conjugates in accordance with some embodiments;

FIG. 17 illustrates H-1 NHS conjugates which can be excited by a blue laser in accordance with some embodiments;

FIG. 18 illustrates H-3 NHS conjugates which can be excited by a blue laser in accordance with some embodiments;

FIG. 19 shows the absorbance and fluorescence spectra of the FRET constructs in accordance with some embodiments;

FIG. 20 illustrates detection of CD45 and isotype on Jurkat cells using goat anti-mouse antibody constructs in accordance with some embodiments;

FIG. 21 illustrates the performance of antibody-FRET pair as a tandem probe in accordance with some embodiments;

FIG. 22 shows detection of live and dead cell populations using Compound N-1 in accordance with some embodiments;

FIG. 23 shows the use of a viability dye in both blue laser based instruments and green laser or yellow laser based instruments in accordance with some embodimetns;

FIG. 24 shows the performance of a viability dye in the cell viability detection where fixation and permeabilization treatments were performed in accordance with some embodiments;

FIG. 25 shows the performance of a viability dye in the yellow channel from a blue and green laser instruments in accordance with some embodiments;

FIG. 26 shows that the percentage of cells and the fluorescence detected were unchanged at 48 hour after fixation in accordance with some embodiments;

FIG. 27 shows the performace of a viability dye in a flow cytometer equipped with a red laser in accordance with some embodiments;

FIG. 28 shows the use of a viability dye in cellular viability experiments in accordance with some embodiments;

FIG. 29 shows the performace of a viability dye in viability assays in accordance with some embodiments;

FIG. 30 shows the performance of a viability dye in the Near-IR channel from a red laser in accordance with some embodiments; and

FIG. 31 shows the good retention of a viability dye in accordance with some embodiments.

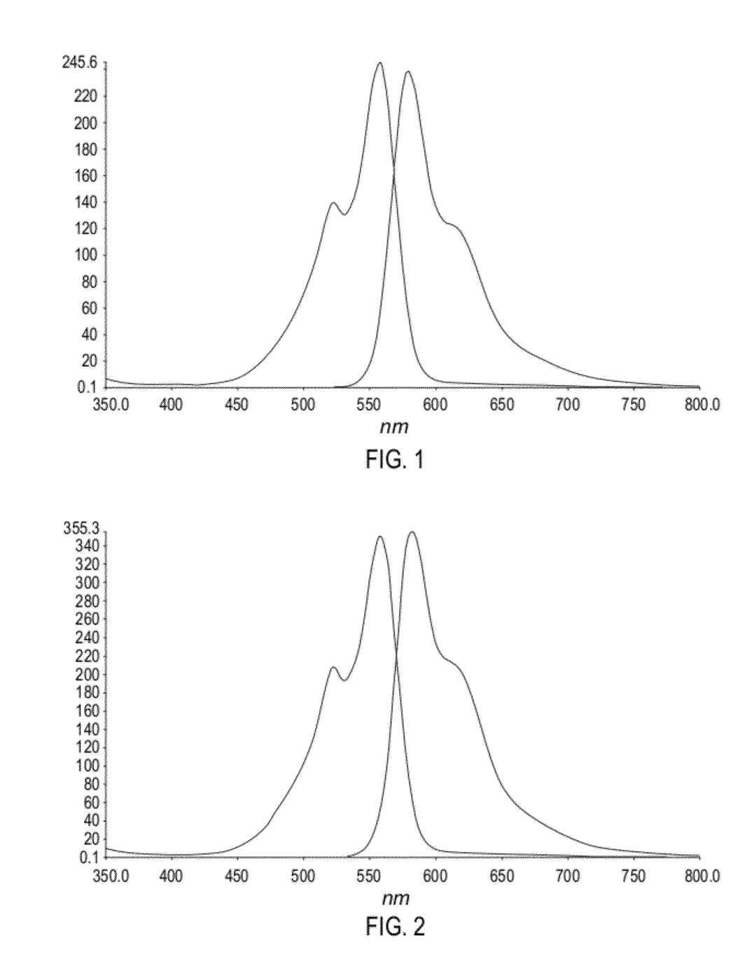

### DETAILED DESCRIPTION

### Definitions

In describing the present invention, the following terms may be employed and are defined as below.

“Conjugates” or “conjugated system” refers to molecular entities in which a group or chain of atoms bears valence electrons that are not engaged in single-bond formation and that modify the behavior of each other. Conjugated polymers are polymers exhibiting such delocalized bonding. Typically conjugated systems can comprise alternating single and double or multiple bonds form conjugated systems, and can be interspersed with atoms (e.g., heteroatoms) comprising nonbonding valence electrons. In some embodiments, conjugated polymers can comprise aromatic repeat units, optionally containing heteroatom linkages.

A “coupling pair” refers to two chemical moieties which can react to form a bond. One or both members of a coupling pair may be activating groups. A member of a coupling pair may be a functional group in a species of interest, and may be formed during initial synthesis or introduced subsequently. Exemplary functional groups include carboxylic and sulfonic acids, amines, hydroxyls, thiols, aldehydes, cyano, and tyrosine. Exemplary bonds that a coupling pair may form include amide, amine, ester, thiol, thioester, disulfide, carbonyl, ether and polymeric linkages.

“Activated” or “activating” as used herein, for example in connection with the terms “group,” “alkyl group” and “carboxylic acid ester,” refers to groups comprising at least one reactive moiety useful for attachment to other molecules (e.g., having available functional groups, for example amino, hydroxy and/or sulfhydryl groups). Exemplary reactive moieties include such groups containing isothiocyanate, isocyanate, monochlorotriazine, dichlorotriazine, mono- or di-halogen substituted pyridine, mono- or di-halo substituted diazine, maleimide, aziridine, sulfonyl halide, acid halide, acid anhydride, hydroxysuccinimide ester, hydroxysulfosuccinimide ester, imido ester, hydrazine, azidonitrophenyl, azide, 3-(2-pyridyl dithio)-proprionamide, glyoxal, aldehyde, and a polymerizable group.

“Multiplexing” herein refers to an assay or other analytical method in which multiple analytes can be assayed simultaneously.

“Optional” or “optionally” means that the subsequently described event or circumstance may or may not occur, and that the description includes instances where said event or circumstance occurs singly or multiply and instances where it does not occur at all. For example, the phrase “optionally substituted alkyl” means an alkyl moiety that may or may not be substituted and the description includes both unsubstituted, monosubstituted, and polysubstituted alkyls.

“Alkyl” refers to a straight or branched or cyclic saturated hydrocarbon group of 1 to 24 carbon atoms optionally substituted at one or more positions, and includes polycyclic compounds. Examples of alkyl groups include optionally substituted methyl, ethyl, n-propyl, isopropyl, n-butyl, s-butyl, t-butyl, n-pentyl, isopentyl, neopentyl, n-hexyl, n-heptyl, n-octyl, n-decyl, hexyloctyl, tetradecyl, hexadecyl, eicosyl, tetracosyl and the like, as well as 10 cycloalkyl groups such as cyclopropyl, cyclobutyl, cyclopentyl, cyclohexyl, cycloheptyl, cyclooctyl, adamantyl, and norbornyl. The term “lower alkyl” refers to an alkyl group of 1 to 6 carbon atoms, preferably 1 to 4 carbon atoms. Exemplary substituents on substituted alkyl groups include hydroxyl, cyano, alkoxy, ═O, ═S, —NO₂, halogen, haloalkyl, heteroalkyl, carboxyalkyl, amine, amide, thioether and —SH.

“Alkoxy” refers to an “—O-alkyl” group, where alkyl is as defined above. A “lower alkoxy” group intends an alkoxy group containing one to six, more preferably one to four, carbon atoms.

“Alkenyl” refers to an unsaturated straight or branched or cyclic hydrocarbon group of 2 to 24 carbon atoms containing at least one carbon-carbon double bond and optionally substituted at one or more positions. Examples of alkenyl groups include ethenyl, 1-propenyl, 2-propenyl (allyl), 1-methylvinyl, cyclopropenyl, 1-butenyl, 2-butenyl, isobutenyl, 1,4-butadienyl, cyclobutenyl, 1-methylbut-2-enyl, 2-methylbut-2-en-4-yl, prenyl, pent-1-enyl, pent-3-enyl, 1,1-dimethylallyl, cyclopentenyl, hex-2-enyl, 1-methyl-1-ethylallyl, cyclohexenyl, heptenyl, cycloheptenyl, octenyl, cyclooctenyl, decenyl, tetradecenyl, 25 hexadecenyl, eicosenyl, tetracosenyl and the like. Preferred alkenyl groups herein contain 2 to 12 carbon atoms. The term “lower alkenyl” intends an alkenyl group of 2 to 6 carbon atoms, preferably 2 to 4 carbon atoms. The term “cycloalkenyl” intends a cyclic alkenyl group of 3 to 8, preferably 5 or 6, carbon atoms. Exemplary substituents on substituted alkenyl groups include hydroxyl, cyano, alkoxy, ═O, ═S, —NO₂, halogen, haloalkyl, heteroalkyl, amine, thioether and —SH.

“Alkenyloxy” refers to an “−O-alkenyl” group, wherein alkenyl is as defined above.

“Alkylaryl” refers to an alkyl group that is covalently joined to an aryl group. Preferably, the alkyl is a lower alkyl. An alkylaryl group may optionally be substituted on either or both of the alkyl and aryl components with substituents, as described herein. Exemplary alkylaryl groups include benzyl, phenethyl, phenopropyl, 1-benzylethyl, phenobutyl, 2-benzylpropyl, 3-naphthylpropenyl and the like.

“Alkylaryloxy” refers to an “−O-alkylaryl” group, where alkylaryl is as defined above.

“Alkynyl” refers to an unsaturated straight or branched hydrocarbon group of 2 to 24 carbon atoms containing at least one —C≡C— triple bond, optionally substituted at one or 10 more positions. Examples of alkynyl groups include ethynyl, n-propynyl, isopropynyl, propargyl, but-2-ynyl, 3-methylbut-1-ynyl, octynyl, decynyl and the like. Preferred alkynyl groups herein contain 2 to 12 carbon atoms. The term “lower alkynyl” intends an alkynyl group of 2 to 6, preferably 2 to 4, carbon atoms, and one —C≡C— triple bond. Exemplary substituents on substituted alkynyl groups include hydroxyl, cyano, alkoxy, ═O, ═S, —NO₂, halogen, haloalkyl, heteroalkyl, amine, thioether and —SH.

“Amide” refers to —C(O)NR′R″ and to —S(O)₂NR′R″, where R′ and R″ are independently selected from hydrogen, alkyl, aryl, and alkylaryl.

“Amine” refers to an —N(R′)R″ group, where R′ and R″ are independently selected from hydrogen, alkyl, aryl, and alkylaryl.

“Aryl” refers to a group that has at least one aromatic ring having a conjugated pi electron system with delocalized pi electrons satisfying Huckel's rule, and includes carbocyclic, heterocyclic, bridged and/or polycyclic aryl groups, and can be optionally substituted at one or more positions. Typical aryl groups contain 1 to 5 aromatic rings, which may be fused and/or linked. Exemplary aryl groups include phenyl, furanyl, azolyl, thiofuranyl, pyridyl, pyrimidyl, pyrazinyl, triazinyl, biphenyl, indenyl, benzofuranyl, indolyl, naphthyl, quinolinyl, isoquinolinyl, quinazolinyl, pyridopyridinyl, pyrrolopyridinyl, purinyl, tetralinyl and the like. Exemplary substituents on optionally substituted aryl groups include alkyl, alkoxy, alkylcarboxy, alkenyl, alkenyloxy, alkenylcarboxy, aryl, aryloxy, alkylaryl, alkylaryloxy, fused saturated or unsaturated optionally substituted rings, halogen, haloalkyl, heteroalkyl, —S(O)R, sulfonyl, —SO₃R, —SR, —NO₂, —NRR′, —OH, —CN, —C(O)R, —OC(O)R, NHC(O)R, —(CH₂)ₙCO₂R or —(CH₂)ₙCONRR′ where n is 0-4, and wherein R and R′ are independently H, alkyl, aryl or alkylaryl.

“Aryloxy” refers to an “−O-aryl” group, where aryl is as defined above.

“Carbocyclic” refers to an optionally substituted compound containing at least one ring and wherein all ring atoms are carbon, and can be saturated or unsaturated.

“Carbocyclic aryl” refers to an optionally substituted aryl group wherein the ring atoms are carbon.

“Halo” or “halogen” refers to fluoro, chloro, bromo or iodo. “Halide,” “fluoride,” “chloride” and the like refer to the anionic form of a halogen when used with reference to a noncovalently bound halogen anion; “acid halide” and the like refers to moieties in which a hydroxyl group of a corresponding acid is replaced with a halogen, typically forming an activated species useful for coupling reactions.

“Haloalkyl” refers to an alkyl group substituted at one or more positions with a halogen, and includes alkyl groups substituted with only one type of halogen atom as well as alkyl groups substituted with a mixture of different types of halogen atoms. Exemplary haloalkyl groups include trihalomethyl groups, for example trifluoromethyl.

“Heteroalkyl” refers to an alkyl group wherein one or more carbon atoms and associated hydrogen atom(s) are replaced by an optionally substituted heteroatom, and includes alkyl groups substituted with only one type of heteroatom as well as alkyl groups substituted with a mixture of different types of heteroatoms. Heteroatoms include oxygen, sulfur, and nitrogen. As used herein, nitrogen heteroatoms and sulfur heteroatoms include any oxidized form of nitrogen and sulfur, and any form of nitrogen having four covalent bonds including protonated and alkylated forms. An optionally substituted heteroatom refers to a heteroatom having one or more attached hydrogens optionally replaced with alkyl, aryl, alkylaryl and/or hydroxyl. The term “lower heteroalkyl” refers to a heteroalkyl group of 1 to 6 carbon and heteroatoms, preferably 1 to 4 carbon and heteroatoms.

“Heterocyclic” refers to a compound containing at least one saturated or unsaturated ring having at least one heteroatom and optionally substituted at one or more positions. Typical heterocyclic groups contain 1 to 5 rings, which may be fused and/or linked, where the rings each contain five or six atoms. Examples of heterocyclic groups include piperidinyl, morpholinyl and pyrrolidinyl. Exemplary substituents for optionally substituted heterocyclic groups are as for alkyl and aryl at ring carbons and as for heteroalkyl at heteroatoms.

“Heterocyclic aryl” refers to an aryl group having at least 1 heteroatom in at least one aromatic ring. Exemplary heterocyclic aryl groups include furanyl, thienyl, pyridyl, pyridazinyl, pyrrolyl, N-lower alkyl-pyrrolo, pyrimidyl, pyrazinyl, triazinyl, tetrazinyl, triazolyl, tetrazolyl, imidazolyl, bipyridyl, tripyridyl, tetrapyridyl, phenazinyl, phenanthrolinyl, purinyl and the like.

“Hydrocarbyl” refers to hydrocarbyl substituents containing 1 to about 20 carbon atoms, including branched, unbranched and cyclic species as well as saturated and unsaturated species, for example alkyl groups, alkylidenyl groups, alkenyl groups, alkylaryl groups, aryl groups, and the like. The term “lower hydrocarbyl” intends a hydrocarbyl group of one to six carbon atoms, preferably one to four carbon atoms.

A “substituent” refers to a group that replaces one or more hydrogens attached to a carbon or nitrogen. Exemplary substituents include alkyl, alkylidenyl, alkylcarboxy, alkoxy, alkenyl, alkenylcarboxy, alkenyloxy, aryl, aryloxy, alkylaryl, alkylaryloxy, —OH, amide, carboxamide, carboxy, sulfonyl, ═O, ═S, —NO₂, halogen, haloalkyl, fused saturated or unsaturated optionally substituted rings, —S(O)R, —SO₃R, —SR, —NRR′, —OH, —CN, —C(O)R, —OC(O)R, —NHC(O)R, —(CH₂)ₙCO₂R or —(CH₂)ₙCONRR′ where n is 0-4, and wherein R and R′ are independently H, alkyl, aryl or alkylaryl. Substituents also include replacement of a carbon atom and one or more associated hydrogen atoms with an optionally substituted heteroatom.

“Sulfonyl” refers to —S(O)₂R, where R is alkyl, aryl, —C(CN)═C-aryl, —CH₂CN, or alkylaryl.

“Thioamide” refers to —C(S)NR′R″, where R′ and R″ are independently selected from hydrogen, alkyl, aryl, and alkylaryl.

“Thioether” refers to —SR, where R is 5 alkyl, aryl, or alkylaryl.

The terms “polynucleotide,” “oligonucleotide,” “nucleic acid” and “nucleic acid molecule” are used herein to include a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms are used interchangeably herein. These terms refer only to the primary structure of the molecule. Thus, these terms include, for example, 3′-deoxy-2′,5′-DNA, oligodeoxyribonucleotide N3′ P5′ phosphoramidates, 2′-O-alkyl-substituted RNA, double and single-stranded DNA, as well as double- and single-stranded RNA, and hybrids thereof including for example hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

Furthermore, modifications to nucleotidic units include rearranging, appending, substituting for or otherwise altering functional groups on the purine or pyrimidine base which form hydrogen bonds to a respective complementary pyrimidine or purine. The resultant modified nucleotidic unit optionally may form a base pair with other such modified nucleotidic units but not with A, T, C, G or U. A basic sites may be incorporated which do not prevent the function of the polynucleotide. Some or all of the residues in the polynucleotide can optionally be modified in one or more ways.

“Nucleic acid probe” and “probe” are used interchangeably and refer to a structure comprising a polynucleotide as defined above that contains a nucleic acid sequence that can bind to a corresponding analyte. The polynucleotide regions of probes may be composed of DNA, and/or RNA, and/or synthetic nucleotide analogs.

“Complementary” or “substantially complementary” refers to the hybridization or base pairing between nucleotides or nucleic acids, such as, for instance, between the two strands of a double stranded DNA molecule or between a polynucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%.

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984). Stringent hybridization conditions will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. Other factors may affect the stringency of hybridization, including base composition and length of the complementary strands, presence of organic solvents and extent of base mismatching, and the combination of parameters used is more important than the absolute measure of any one alone.

“Aptamer” (or “nucleic acid antibody”) is used herein to refer to a single- or double-stranded polynucleotide that recognizes and binds to a molecule of interest by virtue of its shape.

“Polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include polypeptides containing [post-translational] modifications of the polypeptide, for example, glycosylations, acetylations, phosphorylations, and sulphations. In addition, protein fragments, analogs (including amino acids not encoded by the genetic code, e.g. homocysteine, ornithine, D-amino acids, and 25 creatine), natural or artificial mutants or variants or combinations thereof, fusion proteins, derivatized residues (e.g. alkylation of amine groups, acetylations or esterifications of carboxyl groups) and the like are included within the meaning of polypeptide. By “modified” with reference to proteins (including antibodies), and other biomolecules, is meant a modification in one or more functional groups, for example any portion of an amino acid, the structure and/or location of a sugar or other carbohydrate, or other substituents of biomolecules, and can include without limitation chemical modifications (e.g., succinylation, acylation, the structure and/or location of disulfide bonds), as well as noncovalent binding (e.g., of a small molecule, including a drug).

“Amino acid” includes both natural amino acid and substituted amino acids. “Natural amino acid” refers to any of the commonly occurring amino acids as generally accepted in the peptide art and represent L-amino acids unless otherwise designated (with the exception of achiral amino acids such as glycine), including the canonical 20 amino acids encoded directly by the genetic code, as well as selenocysteine, selenomethionine, and ornithine. “Substituted amino acid” refers to an amino acid containing one or more additional chemical moieties that are not normally a part of the amino acid. Such substitutions can be introduced by a targeted deriviatizing agent that is capable of reacting with selected side chains or terminal residues and via other art-accepted methods. For example, cysteinyl residues most commonly are reacted with alpha-haloacetates (and corresponding amines), such as chloroacetic acid or chloroacetamide, to give carboxymethyl or carboxyamidomethyl derivatives. Cysteinyl residues can also be derivatized by reaction with bromotrifluoroacetone, α-bromo-β-(5-imidozoyl)propionic acid, chloroacetyl phosphate, Nalkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Carboxyl side groups (aspartyl or glutamyl) can be selectively modified by reaction with carbodiimides such as 1-cyclohexyl-3-(2-morpholinyl-(4-ethyl) carbodiimide or 1-ethyl-3(4 azonia 4,4-dimethylpentyl) carbodiimide. Aspartyl and glutamyl residues can be converted to asparaginyl and glutaminyl residues by reaction with ammonium ions. Glutaminyl and asparaginyl residues can be deamidated to the corresponding glutamyl and aspartyl residues. Alternatively, these residues can be deamidated under mildly acidic conditions. Other modifications include hydroxylation of proline and lysine, phosphorylation of hydroxyl groups of seryl or theonyl residues, methylation of the alpha-amino groups of lysine, arginine and histidine side chains (see, e.g., T. E. Creighton, Proteins: Structure and Molecule Properties, W. H. Freeman & Co., San Francisco, pp. 79-86 (1983)), acetylation of the N-terminal amine, and amidation of C-terminal carboxyl groups. Blocking groups and/or activating groups can also be incorporated.

As used herein, the term “binding pair” refers to first and second molecules or first and second molecular segments in a molecule that bind specifically to each other with greater affinity than to other components in the sample. The binding between the members of the binding pair is typically noncovalent. Exemplary binding pairs include immunological binding pairs (e.g. any haptenic or antigenic compound in combination with a corresponding antibody or binding portion or fragment thereof, for example digoxigenin and anti-digoxigenin, fluorescein and anti-fluorescein, dinitrophenol and anti-dinitrophenol, bromodeoxyuridine and anti-bromodeoxyuridine, mouse immunoglobulin and goat anti-mouse immunoglobulin), IgG and protein A, IgG and protein G, IgG and protein L, and nonimmunological binding pairs (e.g., biotin and a biotin binding substance [including avidin, streptavidin, or a derivative of either thereof], nucleotides and nucleotide-binding proteins, hormone [e.g., thyroxine and cortisol]-hormone binding protein, receptor-receptor agonist or antagonist (e.g., acetylcholine receptor-acetylcholine or an analog thereof) IgG-protein A, lectin-carbohydrate, enzyme-enzyme cofactor, enzymeenzyme-inhibitor, an organic or inorganic molecule and a biomolecule that binds to the molecule, and two polynucleotides capable of forming nucleic acid duplexes and/or higher order structures) and the like. One or both members of the binding pair can be conjugated to additional molecules.

The term “antibody” as used herein includes antibodies obtained from both polyclonal and monoclonal preparations, as well as: hybrid (chimeric) antibody molecules (see, for example, Winter et al. (1991) Nature 349:293-299; and U.S. Pat. No. 4,816,567); F(ab′)2 and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human hybridomas or from murine hybridomas made from mice expression human immunoglobulin chain genes or portions thereof. See, e.g., Cote, et al. Monoclonal Antibodies and Cancer Therapy, Alan R. Liss, 1985, p. 77.

### Cyanine Compounds

The present invention provides compounds having the general formula I or an isomer, ester, amide, acid halide, acid anhydride, and/or salt thereof, or a mixture of any thereof:

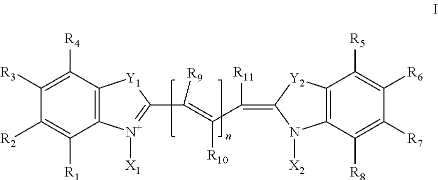

where:

R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;

R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;

Y₁ and Y₂ are independently selected from the group consisting of O, N, S, and —CR′R″— where R′ and R″ are independently H or C₁-C₁₈ alkyl;

X₁ and X₂ are independently selected from the group consisting of optionally substituted alkyl, optionally substituted heteroalkyl, and optionally substituted alkylaryl, wherein at least one of X₁ and X₂ is substituted alkylaryl comprising on the aryl component a substituted alkyl or heteroalkyl comprising a carboxylic acid substituent; and

n is 1, 2, or 3.

The disclosed compounds exhibit optical activity, showing absorption and/or emission of electromagnetic energy. The molecules may desirably fluoresce, and may participate in energy exchange reactions in a variety of formats. The disclosed compounds may desirably exhibit useful properties for a variety of applications, including good solubility in aqueous or predominantly aqueous media, good purification characteristics, ease of conjugation to other substances, and good solubility and purification properties of conjugates thus produced.

Of particular interest are compounds where one or both of X₁ and X₂ are substituted alkylaryl comprising on the aryl component a substituted alkyl or heteroalkyl substituent comprising a carboxylic acid substituent or a derivative thereof. It was found that p-carboxy substitution on an alkylaryl substituent at positions corresponding to X₁ and X₂ could lead to a decrease in cyanine emission. As carboxyl groups impart desirable solubility and coupling properties to these compounds, working embodiments were synthesized to move the carboxyl group from direct attachment to the aryl ring and thereby disrupt any effect resulting from conjugation of the carbonyl moiety with the aryl ring. Moving the carboxyl group from direct attachment to the aryl ring was found to impart fluorescence emission to a corresponding structure that lacked fluorescence when the carboxyl group was directly bound to the aryl group.

Thus, also of interest are those embodiments where one or both of the substituted alkylaryl groups at X₁ and/or X₂ comprise benzyl, phenethyl, or 3-naphthylpropenyl.

The substituted alkylaryl groups are optionally substituted at other positions on their alkyl and aryl components. Other substituents of interest on the substituted aryl moiety of such groups include 1-4 additional groups selected from ═O, ═S, acyl, acyloxy, alkyl, alkenyl, alkynyl, heteroalkyl, optionally substituted alkoxy, optionally substituted amino, optionally substituted aryl, optionally substituted aryloxy, azido, carboxylic acid, (optionally substituted alkoxy)carbonyl, (optionally substituted amino)carbonyl, cyano, halogen, optionally substituted heteroaryl, optionally substituted heteroaryloxy, optionally substituted heterocyclyl, optionally substituted heterocyclooxy, hydroxyl, nitro, sulfanyl, sulfinyl, sulfonyl, sulfonic acid, and a member of a coupling pair. Of particular interest are those aryl substituents selected from alkyl, heteroalkyl, alkoxy, amino alkyl, halo, trihalomethyl, and a member of a coupling pair.

Also of particular interest are embodiments where one or both of Y₁ and Y₂ are O, N, S, or —CR′R″— where R′ and R″ are independently H or C₁-C₁₈ alkyl. It was found that incorporation of O, N, or S at one or both of Y₁ and Y₂ positions can tune the excitation and/or emission wavelengths of the cyanine compounds. Also of interest are those embodiments where n is 1, 2, or 3.

Also of interest are those embodiments where at least one or at least two of R₁ to R₈ are SO₃H, or a derivative thereof. Particular embodiments of interest include those where one or both of R₃ and R₆ are —SO₃H or a derivative thereof. Exemplary derivatives include esters, amides, acid halides, and salts. Sulfonic acids or their derivatives may impart desirable solubility to the cyanine compounds. By varying the number of sulfonic acid group or derivatives thereof on the structure the solubility of the cyanine compounds can be tuned.

In some embodiments provided are compounds of the general formula I where:

R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;

R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;

Y₁ and Y₂ are independently selected from the group consisting of O, N, S, and —CR′R″— where R′ and R″ are independently H or C₁-C₆ alkyl;

n is 1, 2, or 3;

X₁ represents a group having the formula II:

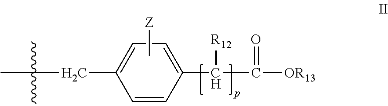

where Z is selected from the group consisting of H, SO₃H, optionally substituted alkyl, and optionally substituted phenyl; p is a number from 1 to 18; R₁₂ is H or C₁-C₁₈ alkyl, and R₁₃ is selected from the group consisting of the formulas III-a, III-b, III-c, III-d, and III-e:

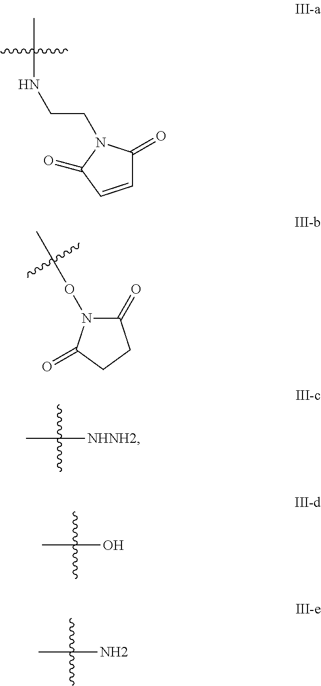

X₂ is the same as X₁, or a group of the formula IV below:

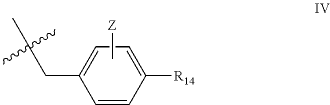

where R₁₄ is an optionally substituted alkyl or optionally substituted phenyl group, and Z is selected from the group consisting of H, SO₃H, optionally substituted alkyl, and optionally substituted phenyl.

In some preferred embodiments provided are compounds of the general formula I where X₁ is a group of the formula II-a:

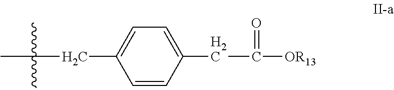

where R₁₃ is selected from the group consisting of the formulas III-a, III-b, III-c, III-d, and III-e shown above, and

X₂ is the same as X₁, or a group of

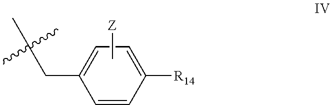

where R₁₄ is an optionally substituted alkyl or optionally substituted phenyl group, and Z is selected from the group consisting of H, SO₃H, optionally substituted alkyl, and optionally substituted phenyl.

In some preferred embodiments provided are compounds of the general formula I where one or both of X₁ and X₂ are a group of the formula II-a, n is 1, 2, or 3, R₃ and R₆ are independently H or SO₃H, and R₁-R₂ and R₆-R₁₁ are independently H.

In some preferred embodiments provided are compounds of the general formula I where n is 1, 2 or 3, R₃ and R₄, and R₅ and R₆ taken together respectively form a 6-membered ring optionally substituted by SO₃H or a derivative thereof, and R₁-R₂ and R₇-R₁₁ are independently H.

Of particular interest are the compounds provided in Table 1. In Table 1R₁₃ is selected from the group consisting of the formulas III-a, III-b, III-c, III-d, and III-e, and R₁₄ is an optionally substituted alkyl or optionally substituted phenyl group.

TABLE 1

| Structure | No. |
|---|---|
| 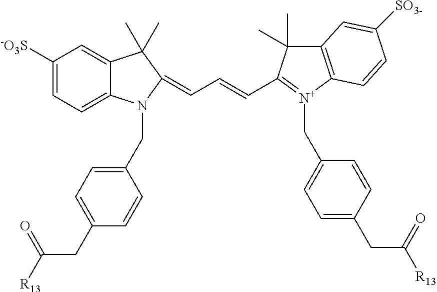 | N-1 |
| 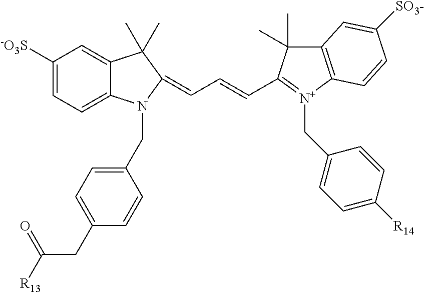 | N-2 |
| 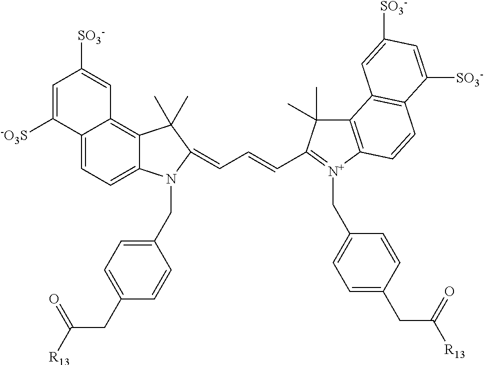 | N-3 |
| 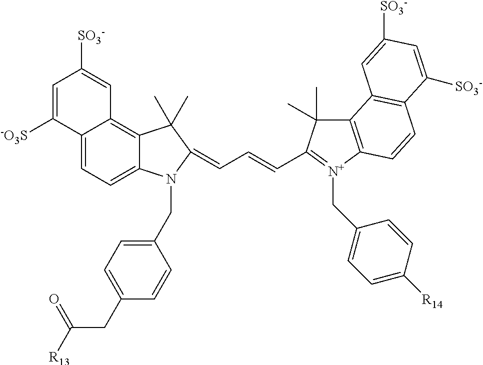 | N-4 |
| 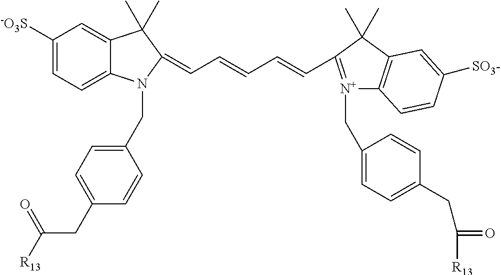 | N-5 |
| 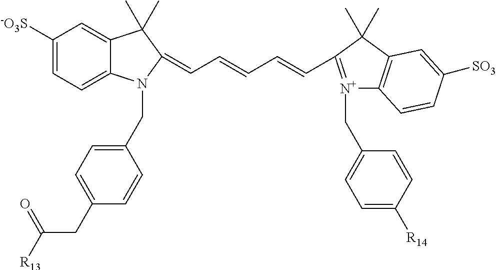 | N-6 |
| 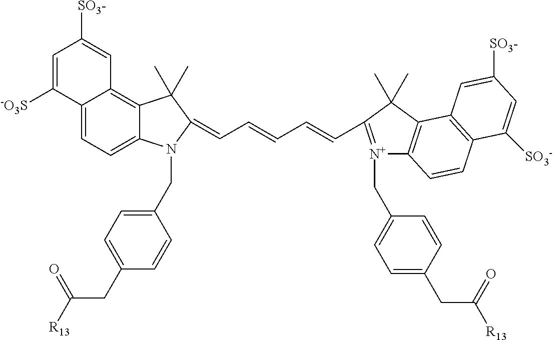 | N-7 |
| 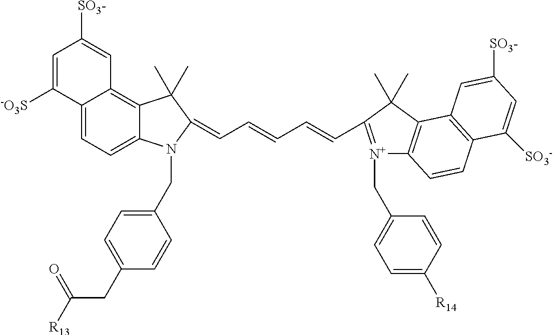 | N-8 |
| 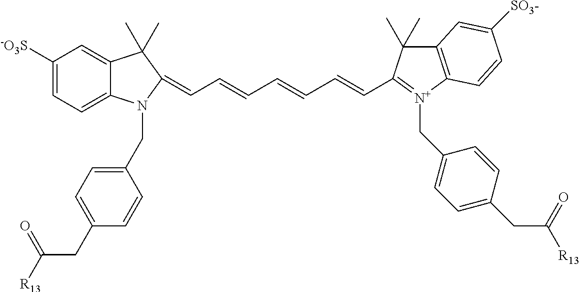 | N-9 |
| 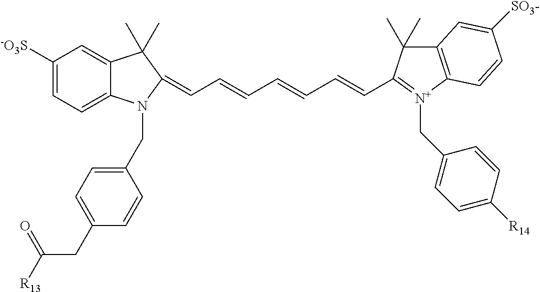 | N-10 |
| 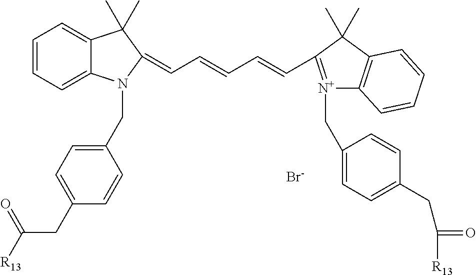 | N-11 |

In some preferred embodiments provided are compounds of the general formula I where X₁ is a group of the formula II-b:

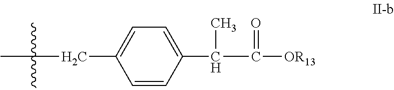

where R₁₃ is selected from the group consisting of the formulas of III-a, III-b, III-c, III-d, and III-e shown above, and

X₂ is the same as X₁ or a group of

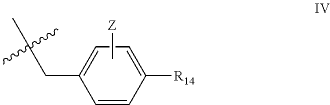

where R₁₄ is an optionally substituted alkyl or optionally substituted phenyl group, and Z is selected from the group consisting of H, SO₃H, optionally substituted alkyl, and optionally substituted phenyl.

In some preferred embodiments provided are compounds of the general formula I where X₁ is a group of the formula II-b, n is 1, 2, or 3, R₃ and R₆ are independently H or SO₃H, and R₁-R₂ and R₆-R₁₁ are independently H.

In some preferred embodiments provided are compounds of the general formula I where X₁ is a group of the formula II-b, n is 1, 2, or 3, R₃ and R₄, and R₅ and R₆ taken together respectively form a 6-membered ring optionally substituted by SO₃H or a derivative thereof, and R₁-R₂ and R₇-R₁₁ are independently H.

Of particular interest are compounds provided in Table 2. In the formulas in Table 2 R₁₃ is selected from the group consisting of the formulas of III-a, III-b, III-c, III-d, and III-e, and R₁₄ is an optionally substituted alkyl or optionally substituted phenyl group

TABLE 2

| Structure | No. |
|---|---|
| 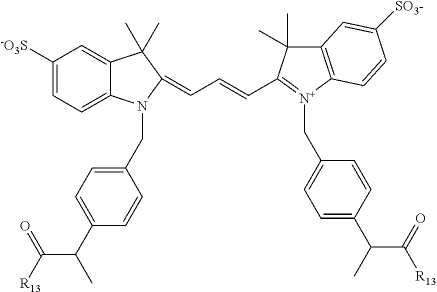 | M-1 |
| 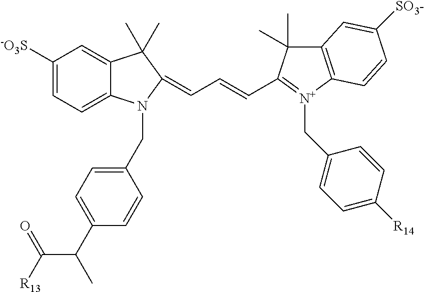 | M-2 |
| 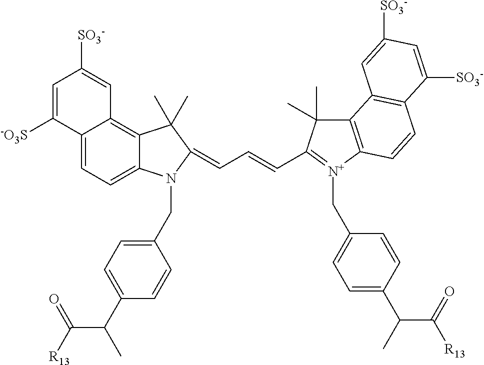 | M-3 |
| 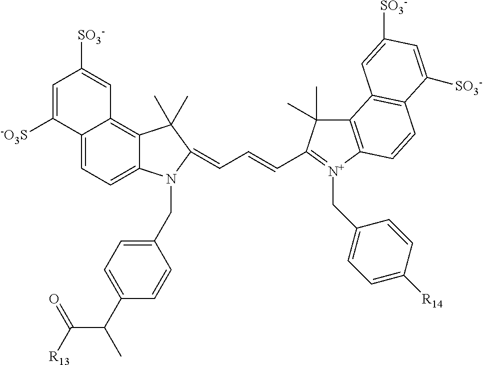 | M-4 |
| 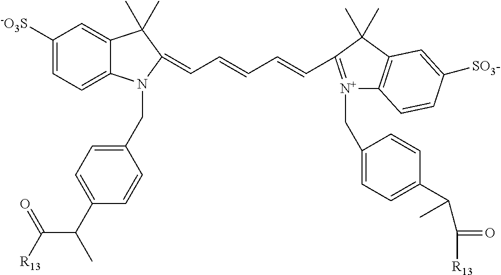 | M-5 |
| 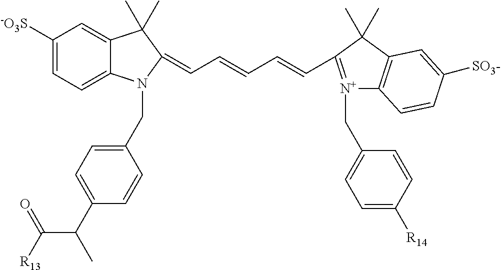 | M-6 |
| 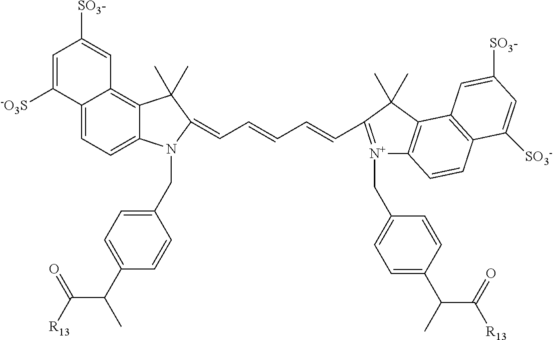 | M-7 |
| 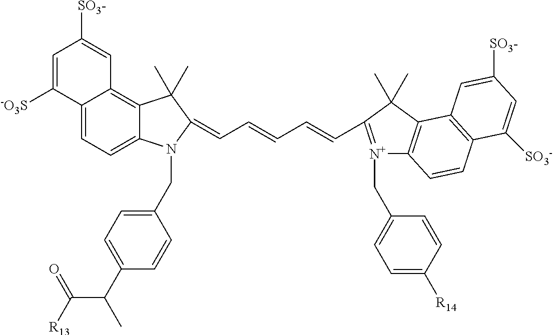 | M-8 |
| 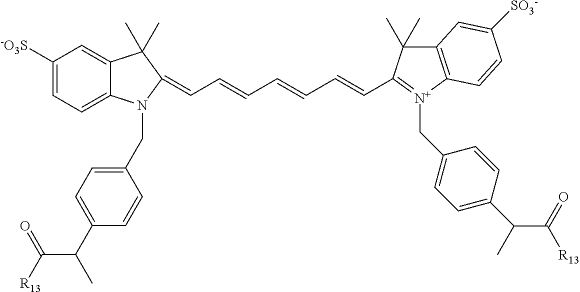 | M-9 |
| 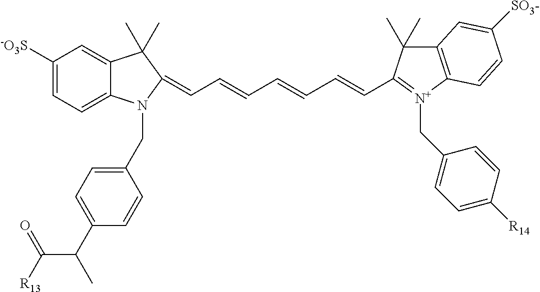 | M-10 |

In some embodiments provided are compounds of the general formula I where:

R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;

R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;

Y₁ and Y₂ are independently selected from the group consisting of O, N, S, and —CR′R″— where R′ and R″ are independently H or C₁-C₁₈ alkyl;

n is 1, 2, or 3; and

X₁ and X₂ are independently selected from the group consisting of NU-1 to NU-30 provided in Table 3. In Table 3, R₁₃ is selected from the group consisting of the formulas III-a, III-b, III-c, III-d, and III-e.

TABLE 3

| Structure | No. |
|---|---|
| 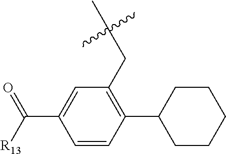 | NU-1 |
| 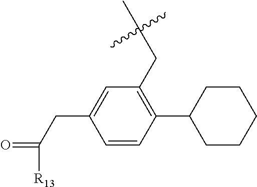 | NU-2 |
| 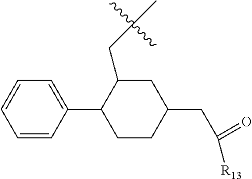 | NU-3 |
| 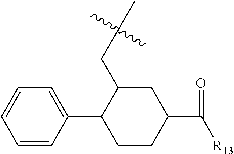 | NU-4 |
| 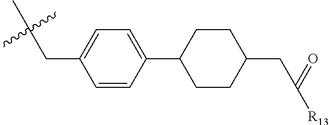 | NU-5 |
| 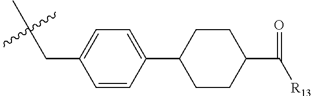 | NU-6 |
| 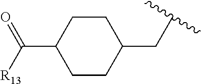 | NU-7 |
| 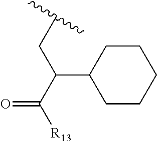 | NU-8 |
| 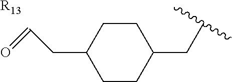 | NU-9 |
| 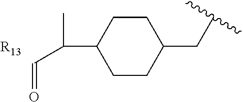 | NU-10 |
| 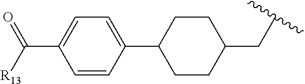 | NU-11 |
| 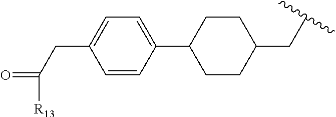 | NU-12 |
| 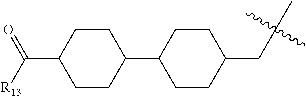 | NU-13 |
| 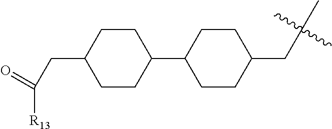 | NU-14 |
| 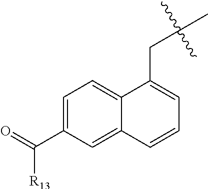 | NU-15 |
| 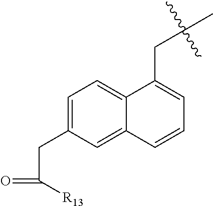 | NU-16 |
| 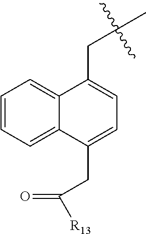 | NU-17 |
| 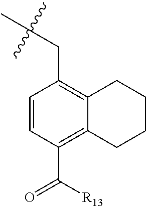 | NU-18 |
| 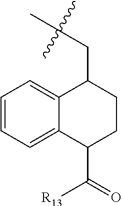 | NU-19 |
| 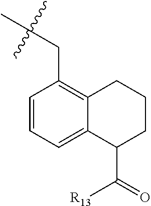 | NU-20 |
| 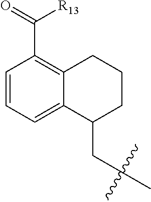 | NU-21 |
| 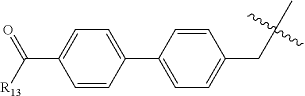 | NU-22 |
| 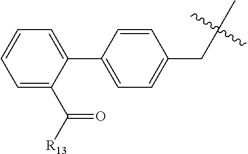 | NU-23 |
| 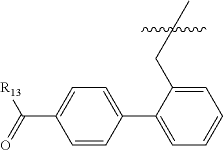 | NU-24 |
| 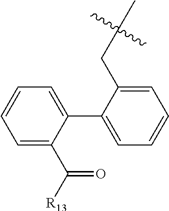 | NU-25 |
| 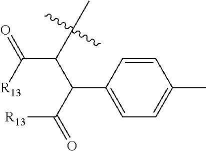 | NU-26 |
| 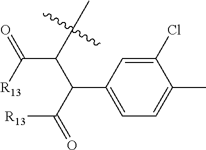 | NU-27 |
| 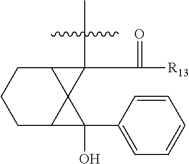 | NU-28 |
| 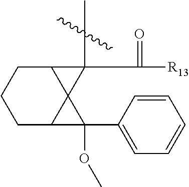 | NU-29 |
| 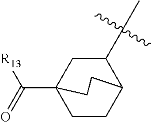 | NU-30 |

Of particular interest are the embodiments where X₁ and X₂ are the same and selected from the group consisting of NU-1 to NU-30 listed in Table 3.

Also of interest are the embodiments where X₁ is selected from the group consisting of NU-1 to NU-30 listed in Table 3, and X₂ is a group of the formula IV:

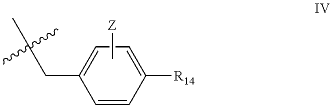

where R₁₄ is an optionally substituted alkyl or optionally substituted phenyl group, and Z is selected from the group consisting of H, SO₃H, optionally substituted alkyl, and optionally substituted phenyl.

In some preferred embodiments compounds having the general formula I-a are provided:

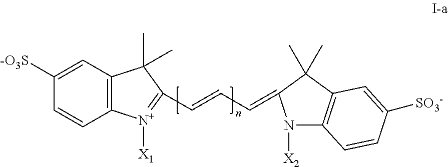

where n is 1, 2, or 3, and X₁ and X₂ are the same and selected from the group consisting of NU-1 to NU-30 listed in Table 3.

In some preferred embodiments compounds having the general formula I-a are provided where X₁ is selected from the group consisting of NU-1 to NU-30 listed in Table 3, and X₂ is a group of the formula IV:

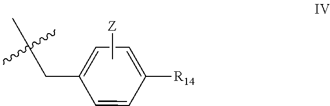

where R₁₄ is an optionally substituted alkyl or optionally substituted phenyl group, and Z is selected from the group consisting of H, SO₃H, optionally substituted alkyl, and optionally substituted phenyl.

In some preferred embodiments compounds having the general formula I-b are provided:

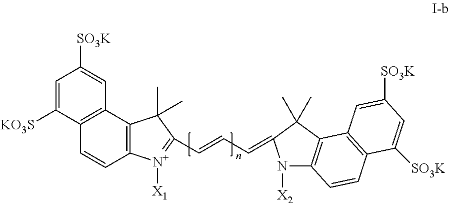

where n is 1, 2, or 3, and X₁ and X₂ are the same and selected from the group consisting of NU-1 to NU-30 listed in Table 3.

In some preferred embodiments compounds having the general formula I-b are provided where X₁ is selected from the group consisting of NU-1 to NU-30 listed in Table 3, and X₂ is a group of the formula IV

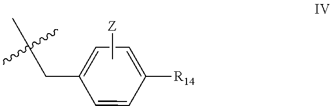

where R₁₄ is an optionally substituted alkyl or optionally substituted phenyl group, and Z is selected from the group consisting of H, SO₃H, optionally substituted alkyl, and optionally substituted phenyl.

The above exemplary compounds having a core structure of formula I-a or formula I-b are provided by way of illustration. It will be appreciated that one or two groups selected from NU-1 to NU-30 shown in Table 3 can be attached to the positions at X₁ and/or X₂ of any core structure having the general formula I.

In some embodiments provided are compounds of the general formula I where:

R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;

R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;

X₁ and X₂ are independently selected from the group consisting of optionally substituted alkyl, optionally substituted heteroalkyl, and optionally substituted alkylaryl, wherein at least one of X₁ and X₂ is substituted alkylaryl comprising on the aryl component a substituted alkyl or heteroalkyl comprising a carboxylic acid substituent;

n is 1, 2, or 3; and

Y₁ and Y₂ are independently selected from the group consisting of O, N, S, and —CR′R″— where R′ and R″ are independently H or C₁-C₁₈ alkyl, and at least one of Y₁ and Y₂ is O, S, or N.

Of particular interest are compounds of the general formula I where both of Y₁ and Y₂ are O, and X₁ and X₂ are the same and represent a group of the formula II.

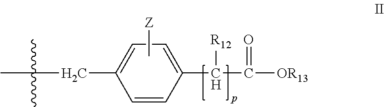

where Z is selected from the group consisting of H, SO₃H, optionally substituted alkyl, and optionally substituted phenyl; p is a number from 1 to 18; R₁₂ is H or C₁-C₆ alkyl, and R₁₃ is selected from the group consisting of the formulas of III-a, III-b, III-c, III-d, and III-e shown above.

Also of particular interest are compounds of the general formula I where both of Y₁ and Y₂ are O, one of X₁ and X₂ is a group of the formula II, and one of X₁ and X₂ is a group of the formula IV:

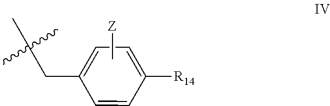

where R₁₄ is an optionally substituted alkyl or optionally substituted phenyl group, and Z is selected from the group consisting of H, SO₃H, optionally substituted alkyl, and optionally substituted phenyl.

Also of particular interest are compounds of the general formula I where one of Y₁ and Y₂ is O, one of Y₁ and Y₂ is C(CH₃)₂, and X₁ and X₂ are the same and represent a group of the formula II.

Also of particular interest are compounds of the general formula I where one of Y₁ and Y₂ is O, one of Y₁ and Y₂ is C(CH₃)₂, one of X₁ and X₂ is a group of the formula II, and one of X₁ and X₂ is a group of the formula IV.

In some preferred embodiments provided are compounds of the general formula I wherein one or both of Y₁ and Y₂ are O, and at least one of R₁ to R₈ is SO₃H, or an isomer, ester, amide, acid halide, and/or salt thereof, or a mixture of any thereof.

In some preferred embodiments provided are compounds of the general formula I wherein one or both of Y₁ and Y₂ are O, R₃ and R₄, and R₅ and R₆ taken together respectively form a 6-membered ring optionally substituted by SO₃H or a derivative thereof, and R₁-R₂ and R₇-R₁₁ are independently H.

In some preferred embodiments provided are compounds having the general formula I-c:

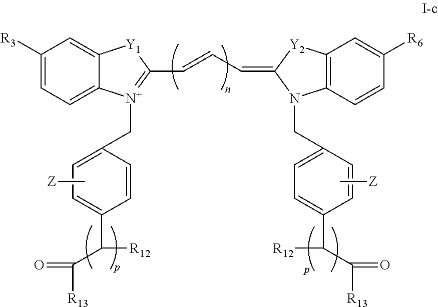

where:

R₃ and R₆ are independently H or SO₃H;

Y₁ and Y₂ are independently O, N, S, or —CR′R″— where R′ and R″ are independently H or C₁-C₁₈ alkyl,

n is 1, 2, or 3,

Z is selected from the group consisting of H, SO₃H, optionally substituted alkyl, and optionally substituted phenyl;

p is a number from 1 to 18;

R₁₂ is H or C₁-C₆ alkyl; and

R₁₃ is selected from the group consisting of the formulas III-a, III-b, III-c, III-d, and III-e:

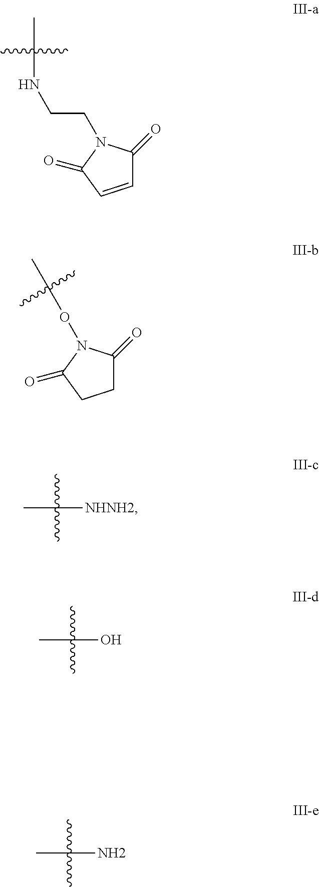

In some preferred embodiments both of Y₁ and Y₂ in the compounds of the general formula I-c are independently O, N, or S. In some embodiments one of Y₁ and Y₂ is O, N, S and one of Y₁ and Y₂ is —CR′R″— where R′ and R″ are independently H or C₁-C₁₈ alkyl. In some embodiments both of Y₁ and Y₂ are —CR′R″— where R′ and R″ are independently H or C₁-C₁₈ alkyl.

In some preferred embodiments provided are compounds having the general formula I-d:

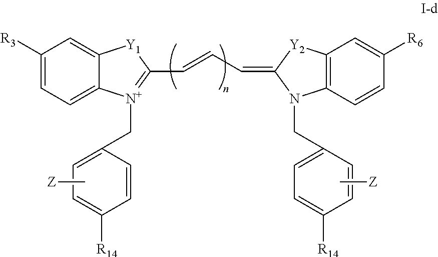

where:

R₃ and R₆ are independently H, optionally substituted alkyl, optionally substituted phenyl;

Y₁ and Y₂ are independently O, N, S, or —CR′R″— where R′ and R″ are independently H or C₁-C₁₈ alkyl;

n is 1, 2, or 3,

Z is selected from the group consisting of H, optionally substituted alkyl, and optionally substituted phenyl; and

R₁₄ is an optionally substituted alkyl or optionally substituted phenyl group.

In some preferred embodiments both of Y₁ and Y₂ in the compounds of the general formula I-d are independently O, N, or S. In some embodiments one of Y₁ and Y₂ is O, N, or S, and one of Y₁ and Y₂ is —CR′R″— where R′ and R″ are independently H or C₁-C₁₈ alkyl. In some embodiments both of Y₁ and Y₂ are —CR′R″— where R′ and R″ are independently H or C₁-C₁₈ alkyl.

Table 4 provides exemplary compounds of particular interest. In Table 4, R₁₃ is selected from the group consisting of formulas III-a, III-b, III-c, III-d, and III-e shown above, and R₁₄ is an optionally substituted alkyl or optionally substituted phenyl group.

TABLE 4

| | |
|---|---|
| 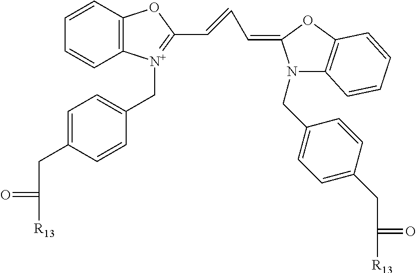 | H-1 |
| 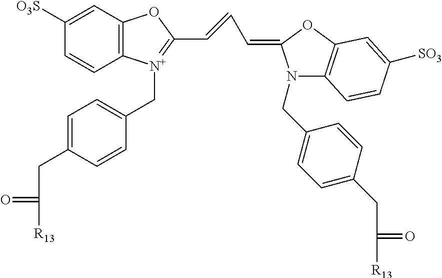 | H-2 |
| 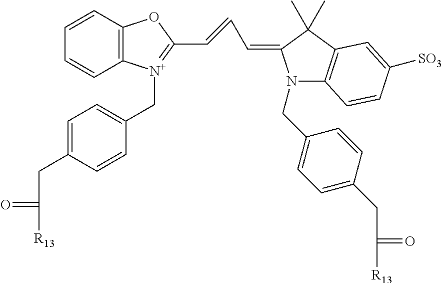 | H-3 |
| 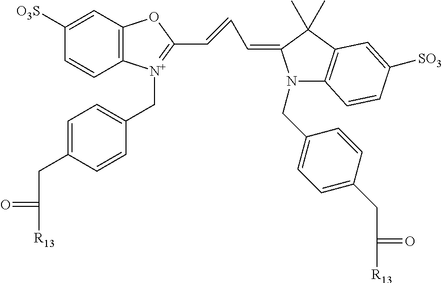 | H-4 |
| 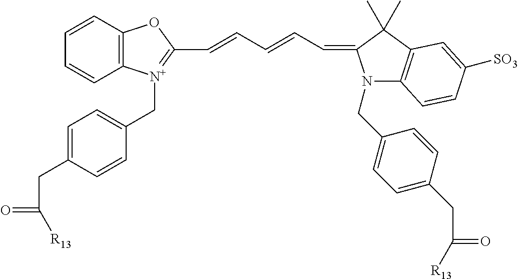 | H-5 |
| 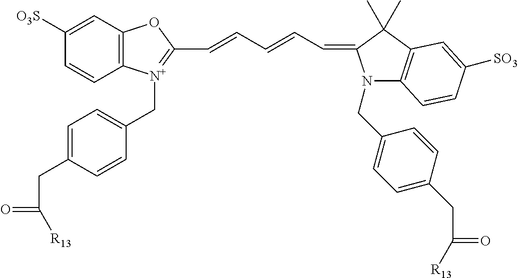 | H-6 |
| 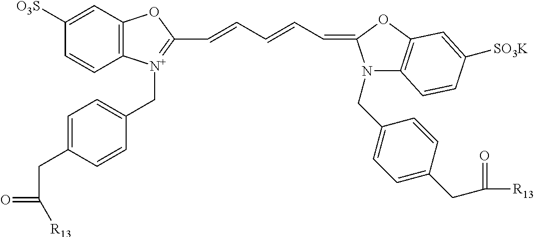 | H-7 |
| 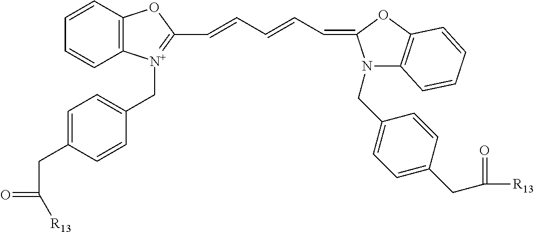 | H-8 |
| 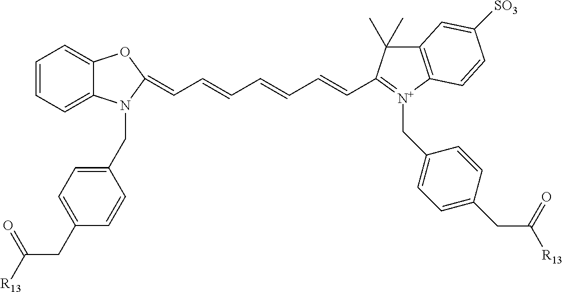 | H-9 |
| 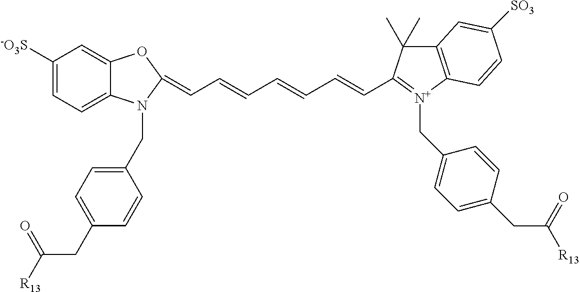 | H-10 |
| 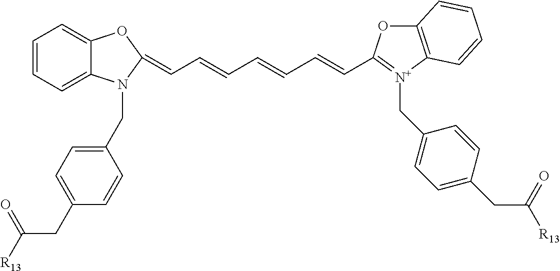 | H-11 |
| 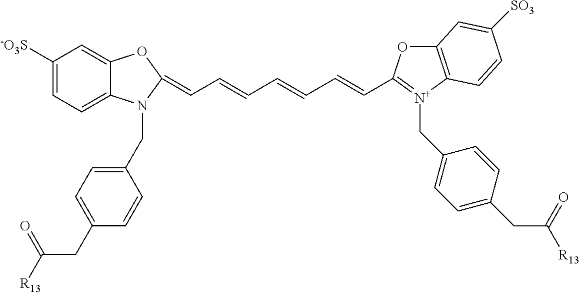 | H-12 |
| 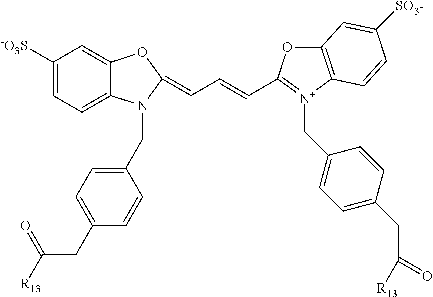 | H-13 |
| 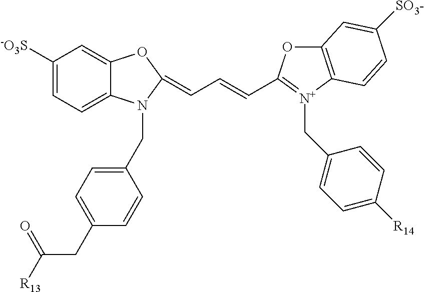 | H-14 |
| 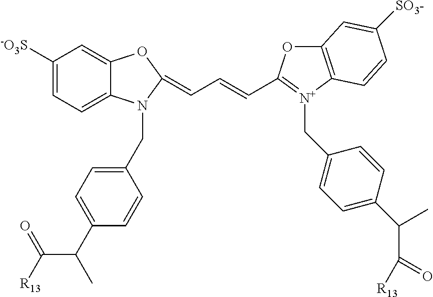 | H-15 |
| 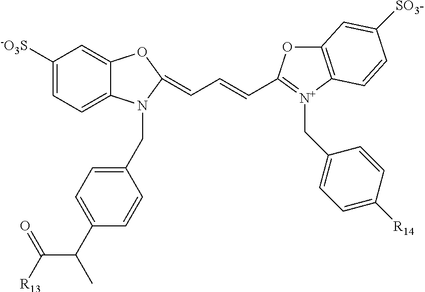 | H-16 |
| 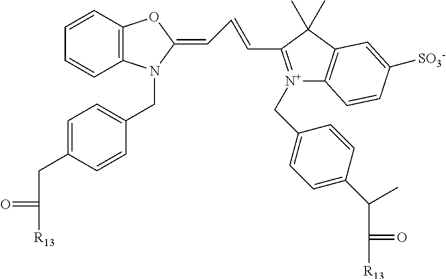 | H-17 |

The compounds of the invention are desirably optically active, and exhibit absorption and/or emission properties. The compounds can thus be utilized in applications where absorption of energy of particular wavelengths is desired, in applications where emission of energy is desired, and in applications where both absorption and emission are desired. The compounds may be used as labels for substances including biomolecules by coupling or recruiting the compounds to particular substances and/or locations. The compounds may be used to form conjugates with other substances. The compounds may be used in energy transfer experiments, and may be provided in transfer complexes or in forms which can be recruited to the location of other optically active substances with which they exhibit energy transfer. The compounds can be used as photographic sensitizers, in dye lasers, as saturable absorbers for passively switching lasers, and as molecular probes of membrane potential. The compounds may be used in a variety of applications in which labels of biomolecules are used, including in labeling of primary, secondary (or subsequent) antibodies, in labeling of nucleotides that can be incorporated into labeled polynucleotides, including sequencing reactions (e.g., in labeled nucleotides and dye-terminators), in proximity assays used to determine the proximity of two optically active substances in a variety of settings, in apoptotic assays, in photobleaching recovery experiments, in fluorescence correlation spectroscopy, in microarray experiments, in transcriptomics, and in proteomics. Desirably, the compounds of the invention exhibit good solubility in aqueous media.

The compounds may be provided as isolated compounds, as solvates, as solutions, as conjugates, and in other forms as described. Also provided are compositions and articles that comprise the compounds in an excited state, attained either by direct excitation with an electromagnetic source or by energy transfer from another excited species. These articles include conjugated sensors as well as detection complexes employing excited cyanines.

Salts of the described compounds can be prepared through techniques known in the art. By “exchanging,” “replacing,” “substituting” and the like with relation to the counterions associated with a cyanine is meant exchanging at least 80% of the associated counterions at the desired position. Preferably at least 85% of the counterions are exchanged, more preferably at least 90%, and most preferably 95% or more of the counterions are exchanged. In some cases there may be no detectable levels of the original counterions associated with the compound. Counterion association can be determined by any suitable technique, for example by XPS spectroscopy and/or mass spectrometry. The counterions may be exchanged by any appropriate method known or discoverable in the art. Exemplary ion exchange methods include mass action, dialysis, chromatography, and electrophoresis. After counterion exchange, the new salt form(s) of the cyanine can optionally be purified and/or isolated. Any suitable method(s) that leads towards the purification and/or isolation of the salt of interest can be used. Exemplary methods include crystallization, chromatography (e.g., exclusion, HPLC, FPLC), precipitation, and extraction. Similarly, esters, amides and acid halides can be formed from the described compounds, and purified and/or characterized if desired, using known techniques.

Also of interest are the solvates and solutions produced by dissolving the compounds of the invention in a solvent or solvent mixture. In some embodiments, the cyanines described herein are soluble in aqueous solutions and other highly polar solvents, and can be soluble in water. By “water-soluble” is meant that the material exhibits solubility in a predominantly aqueous solution, which, although comprising more than 50% by volume of water, does not exclude other substances from that solution, including without limitation buffers, blocking agents, cosolvents, salts, metal ions and detergents. Additional solvents which may be used to form solutions, either alone or in combination, include DMSO, DMF, and lower alcohols. Solutions may be provided in a container of any suitable form. Solutions may be packaged in a container designed for incorporation into a solution processing apparatus, for example a printer. In some embodiments, the solution may be provided in an inkjet cartridge designed to be used with a printing device.

### Conjugates

Conjugates of the cyanine compounds are provided by coupling one or more disclosed cyanine compounds to one or more other substances. Exemplary conjugates of interest include those comprising a disclosed cyanine and a biomolecule, a substrate, a probe, a linker, a target, a low affinity false target, a small molecule, one member of a binding pair, a polymer and/or an optically active species (particularly one with which the cyanine may exchange energy), inert surfaces, beads, nanoparticles etc., or a combination of any thereof. Advantegously the labeled biomolecular probes can be used for detection of cells, proteins, metabolite, nucleic acids etc. and used as markers.

Probes and targets form members of binding pairs as described. Targets can include cells, cell fragments, and cell surface molecules, for example immune system molecules, receptors, and markers indicative of specific cell populations or subpopulations. Biomolecules include any species or mixture of species that can be produced by or obtained from a living organism, including cell or bacterial cultures. Exemplary biomolecules include proteins, peptides, polynucleotides, polysaccharides, antibodies, triglycerides, lipoproteins, and lectins.

One or more probes may be employed that bind to particular species of targets. The probe and the target may form a binding pair that specifically binds to each other. A sensor biomolecule can be used as a probe that can bind to a target biomolecule. A sensor polynucleotide can be branched, multimeric or circular, but is typically linear, and can contain normatural bases. The sensor may be a peptide nucleic acid, the molecular structures of which are well known.

Any polymer can be used to form a conjugate either as a discrete entity or by incorporation into another material of interest, including incorporation on or into a substrate. Exemplary polymers of interest include hydrocarbon polymers (e.g., formed from optionally substituted alkenes and/or optionally substituted alkynes), hydrophilic polymers, heteroalkyl polymers, including polyalkylene oxides including polytheylene oxide and polypropylene oxide, polyamines, and dendrimers. Polymers may be or may incorporate other optically active species.

In some instances, the polymer and/or biomolecule can serve as a carrier for a cyanine of the invention, and may prolong its halflife when used in a physiological setting. For example, a cyanine may be coupled to a serum albumin (e.g., bovine, rabbit, mouse, human), a globulin (e.g., alpha, beta, gamma or immunoglobulins), or a hydrophilic polymer (e.g., a polyalkylene oxide such as polyethylene glycol, polypropylene glycol, or a copolymer thereof).

Optically active species of interest include those with which the cyanine can exchange energy, either directly or through intermediate species. Optically active species can include synthetic dyes, semiconductor nanocrystals, lanthanide chelates, polymers, proteins and/or optically active fragments thereof. Exemplary proteins include green fluorescent protein, alternatively colored derivatives thereof, Renilla luciferase, phycoerythrin (PE), phycoerythrin B, phycoerythrin R, B or Y, phycocyanin, and allophycocyanin (APC), and derivatives of any thereof. Some particular conjugates of interest include PE-APC-NGy7, PE-NGy5, and NGy3-APC. Conjugates may include more than one additional optically active species.

Conjugates can be formed by reaction of moieties on their components, and may include linking groups useful for coupling and/or spacing the components appropriately. The components which are used as precursors for the conjugates include or are derivatized to include one or more members of coupling pairs that can react with corresponding members of coupling pairs on other conjugate components. Appropriate blocking strategies can be used as needed to protect functional groups that are not to be used in conjugate formation, as known in the art.

Exemplary coupling schemes include amine coupling, thiol coupling, aldehyde coupling, tyrosine coupling, polymeric coupling, and bifunctional (or polyfunctional) crosslinking agents. Amine coupling can be accomplished through reaction of an amine group on one component with an activated ester on another component (for example an N-hydroxysuccinimide ester). Thiol coupling can be accomplished by reaction of an activated thiol group (e.g., a 2-pyridinyldithio moiety) on one component with a thiol group on another component. Alternatively, a thiol group on one component can be reacted with a maleimide or iodoacetyl group on another component. Aldehyde coupling can be accomplished by reaction of an aldehyde group on one component with a hydrazide group on another component. Tyrosine groups can be coupled to diazo groups. Polymeric coupling can be accomplished by preparing a derivatized monomer or repeat unit linked to a component of interest and then performing a polymerization reaction that incorporates that derivatized monomer. The coupling members may be natively present on the species to be coupled, or may be introduced; chemical schemes for introducing such groups are known. For example, aldehyde groups can be introduced by oxidation of cis-diols (as found in many polyols including sugars, polysaccharides and glycoconjugates) with sodium metaperiodate. Bifunctional and heterobifunctional crosslinking agents can also be used to link functional groups that cannot be otherwise directly; for example, glutaraldehyde may be used to crosslink two amine groups, and maleimide hydrazide can be used to link thiol and formyl groups (Heindel et al., Bioconj. Chem. 2(6):427-30, 1997, November-December).

“Linking groups,” or “linkers,” can be conjugated to the cyanines of the invention, and can be used to conjugate any of the species of the conjugate to each other. The particular composition of the linking group is not critical. Exemplary linking groups include alkyls, heteroalkyls (e.g., polyethers, alkylamines, polyamines), aryls, heteroaryls, alkylaryls, synthetic polymers, naturally occurring polymers, amino acids, a carbohydrates, polypeptides, or combinations thereof, each optionally substituted as described herein with regard to the components of the linking group. In some embodiments, a linking group may be symmetric, rigid and/or sterically hindered, or may comprise a region having one or more of these properties. The linker may be designed to impose a separation distance suitable for energy transfer between two species to which it is coupled, for example imparting a separation from about 10 to about 100 angstroms. The linker may impose a distance of less than about 100 angstroms, less than about 70 angstroms, less than about 30 angstroms, or less than about 20 angstroms. The linking group may comprise one or more different members of coupling pairs, and typically contains at least two members of a coupling pair to allow for linking of at least two substances.

In some embodiments, the cyanine may be deposited on, coupled or otherwise linked to a substrate. The substrate can comprise a wide range of material, either biological, nonbiological, organic, inorganic, or a combination of any of these. In some embodiments, the substrate can be transparent. The substrate can be a rigid material, for example a rigid plastic or a rigid inorganic oxide. The substrate can be a flexible material, for example a transparent organic polymer such as polyethyleneterephthalate or a flexible polycarbonate. The substrate can be conductive or nonconductive.

The cyanine compounds can be deposited on a substrate in any of a variety of formats. For example, the substrate may be a polymerized Langmuir Blodgett film, functionalized glass, Si, Ge, GaAs, indium doped GaN, GaP, SiC (Nature 430:1009, 2004), SiO2, SiN4, semiconductor nanocrystals, modified silicon, or any of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), 20 polyethyleneterephthalate, polysiloxanes, polymeric silica, latexes, dextran polymers, epoxies, polycarbonates, agarose, poly(acrylamide) or combinations thereof. Conducting polymers and photoconductive materials can be used. The substrate can take the form of a photodiode, an optoelectronic sensor such as an optoelectronic semiconductor chip or optoelectronic thin-film semiconductor, or a biochip.

In some embodiments, the substrate may be particles that are non-uniform/irregular in shape. The particles may have at least two different (X-, Y- and/or Z-) dimensions, and may have three (or more, for unusually shaped particles) different dimensions. The particles are therefore nonspherical, having a shape other than that of a solid sphere. In some embodiments, the particles exhibit an increased surface area over a sphere or other solid shape occupying the same volume. Desirably, the non-uniform particles exhibit an irregular surface (on a macro- and/or micro-scale) that produces a large increase in surface area. The particles desirably exhibit at least a two-fold increase in surface area, and may exhibit at least a three-fold, five-fold, 10-fold or 20-fold increase in surface area. The particles may exhibit up to a 30-fold, 40-fold, 50-fold, 100-fold, or 200-fold increase in surface area over a similarly sized smooth spherical particle. The particles m 5 ay exhibit an increased binding capacity over a similarly-sized spherical particle, which may result from the increased surface area and/or from an increase in the density of capture moieties (or derivatizable functionalities) used to bind analyte. Desirably, at least one, two or three (or all) dimensions of the particle may be less than about 30 or 40 microns, as is compatible with flow cytometric systems, and may be less than about 20 microns, less than about 10 microns, or less than about 2 microns in such dimensions. With reference to these dimensions, it is understood that such particles are typically provided as distributions of different sizes, and that particles will exhibit mean distributions meeting this limitation, such that an average particle in a population will meet such limitation(s). The particles may be generally bead like, although lacking a uniform spherical surface, and may be porous, microporous or macroporous, or may be nonporous. Particles having a mean diameter of less than 2 microns may be desirable, as they can exhibit improved suspension properties which can lead to increased contact with the test sample and/or higher binding capacities.

Conjugates can comprise more than one additional substance in addition to the cyanine. For example, a conjugate may comprise a cyanine, an optically active species with which the cyanine can exchange energy, and a probe or sensor for a target of interest. Such a conjugate may also include a low affinity false target, which blocks the probe/sensor component in the absence of the target of interest and binds at a lower affinity than the target, and thereby can reduce or eliminate background signals formed from spurious binding of the probe/sensor region in the absence of target.

### Articles of Manufacture

The disclosed cyanine compounds can be incorporated into articles of manufacture described herein as well as in articles in which cyanines have previously been used. Exemplary articles of manufacture include conjugates such as derivatized particles or beads, derivatized members of binding pairs, antibody conjugates, derivatized small molecules, biosensors, stains, and can be used in array or microarray form. The cyanines may be used in holographic gratings in combination with synthetic polymers (e.g., vinyl polymers) in information storage devices, for example in CD-R and DVD-R media.

Cyanine labeled species (probes and/or targets) can form detection complexes incorporating the probe, its target, and one or more conjugated cyanines. Also provided are compositions and articles comprising cyanines of the invention in an excited state, attained either by direct excitation with an electromagnetic source or by energy transfer from one or a series of different molecules. These articles include conjugated sensors as well as detection complexes comprising excited cyanines.

Solution processing methods can be used to incorporate cyanines into articles of manufacture where appropriate. Printing techniques may advantageously be used to deposit the cyanines in certain settings, e.g., inkjet printing, offset printing, etc. Where desired, after deposition of a solution comprising a cyanine, the solvent can be removed. Any available method or combination of methods may be used for removing the solvent. Exemplary solvent removal methods include evaporation, heating, extraction, and subjecting the solution to a vacuum, and combinations comprising any thereof.

Embodiments of the invention include articles of manufacture utilizing cyanines of the invention. For example, a plurality of labeled sensors comprising cyanines can be used simultaneously in an array. Multiplex embodiments may employ 2, 3, 4, 5, 10, 15, 20, 25, 50, 100, 200, 400, 1000, 5000, 10000, 50000 or more distinct articles incorporating one or 20 more embodiments described herein. Other aspects of the invention are discussed further herein.

### Methods of Use

The cyanine compounds described herein can be used in a variety of methods, as known for other cyanines and other fluorescent compounds. The cyanine compounds may be used for direct labeling, detection, and/or quantitation of a substance of interest. The cyanine compounds can be used in binding assays, including competitive binding assays, by labeling one member of a binding pair with a cyanine. The cyanines can be bound to a substrate directly or through one or more intermediate species. Conjugated species including conjugated particles can be used for the detection and/or quantitation of a target analyte.

Exemplary methods of use include cytometric settings, sequencing of polynucleotides using for example singly or multiply-labeled nucleotides and/or dye terminators, microarray and nanoarray labeling, coding schemes, and energy transfer experiments. The cyanines may be used in bead-based assays and/or cellular assays. Other biological applications in which cyanines can be used include comparative genomic hybridization, transcriptomics, and proteomics, and as markers in microscopic applications. The cyanines of the invention can serve as donors, acceptors, or both, including in multiple energy transfer schemes in which cyanine(s) of the invention form one or more components.

The cyanines may be used in methods which screen for a property of interest. For example, the materials may be tested for increased fluorescent efficiency, for absorbance wavelength, emission wavelength, conductive properties, and other properties described herein. Cyanines can be used to increase the sensitivity range of photographic emulsions.

In some embodiments, methods of analyzing a sample for a target are provided, comprising providing a sample suspected of containing a target, contacting the sample with a conjugate comprising a cyanine and a probe or sensor under conditions in which the probe can bind to the target, if present, to form a detection complex, contacting the sample or a fraction thereof suspected of comprising the detection complex with an energy source that can be absorbed by or transferred to the compound, and determining if energy has been absorbed or transferred to the complex. Such assays may also include low affinity false targets in the conjugate and/or in the detection complex that can be displaced from the probe region by binding of the conjugate to the actual target.

The target analyte in such assays may be a biomolecule, for example a peptide or protein, a polynucleotide such as DNA or RNA, an antibody, saccharides, oligosaccharides, polysaccharides, etc. Alternatively, the target analyte may be a small molecule, and may be organic or inorganic.

In some embodiments, the sample or portion of the sample comprising or suspected of comprising the analyte can be any source of biological material, including cells, tissue or fluid, including bodily fluids, and the deposits left by that organism, including viruses, mycoplasma, and fossils. Typically, the sample is obtained as or dispersed in a predominantly aqueous medium. Nonlimiting examples of the sample include blood, urine, semen, milk, sputum, mucus, a buccal swab, a lavage, a vaginal swab, a rectal swab, an aspirate, a needle biopsy, a section of tissue obtained for example by surgery or autopsy, plasma, serum, spinal fluid, cerebrospinal fluid, amniotic fluid, lymph fluid, the external secretions of the skin, respiratory, intestinal, and genitourinary tracts, tears, saliva, tumors, organs, samples of in vitro cell culture constituents (including but not limited to conditioned medium resulting from the growth of cells in cell culture medium, putatively virally infected cells, recombinant cells, and cell components, including without limitation hybridoma supernatants producing human or murine antibodies and supernatants from cells producing fragments or modified forms of antibodies or other immunological or secreted proteins), a cellular lysate, and a recombinant library comprising polynucleotide sequences.

The sample can be a positive control sample which is known to contain the analyte. A negative control sample can also be used which, although not expected to contain the analyte is suspected of containing it, and is tested in order to confirm the lack of contamination by the target analyte of the reagents used in a given assay, as well as to determine whether a given set of assay conditions produces false positives (a positive signal even in the absence of analyte in the sample). The sample can be diluted, dissolved, suspended, purified, extracted or otherwise treated to solubilize or resuspend any target analyte present or to render it accessible to reagents.

### Excitation and Detection

Any instrument that provides a wavelength that can excite the cyanine and/or a species with which the cyanine can exchange energy and is shorter than the emission wavelength(s) to be detected can be used for excitation. Commercially available devices can provide suitable excitation wavelengths as well as suitable detection components. Any electromagnetic emission wavelength that can be produced and detected can be used.

Exemplary excitation sources include a broadband UV light source such as a deuterium lamp with an appropriate filter, the output of a white light source such as a xenon lamp or a deuterium lamp after passing through a monochromator to extract out the desired wavelength(s), a continuous wave (cw) gas laser, a solid state diode laser, or any of the pulsed lasers. Emitted light can be detected through any suitable device or technique; many suitable approaches are known in the art.

Incident light wavelengths useful for excitation can include 300 nm to 1000 nm wavelength light. Exemplary useful incident light wavelengths include, but are not limited to, wavelengths of at least about 300, 350, 400, 450, 500, 550, 600, 700, 800 or 900 nm, and may be less than about 1000, 900, 800, 700, 600, 550 or 500 nm. Exemplary useful incident light in the region of 450 nm to 500 nm, 500 nm to 550 nm, 550 nm to 600 nm, 600 nm to 700 nm, and 700 nm to 1000 nm. In certain embodiments, the complexes form an excited state upon illumination with incident light including a wavelength of about 488 nm, about 532 nm, about 594 nm and/or about 633 nm. Additionally, useful incident light wavelengths can include, but are not limited to, 488 nm, 532 nm, 594 nm and 633 nm wavelength light.

Any apparatus that can detect an emission produced from a cyanine or a species to which energy has been transferred may be used, including without limitation microscopes, spectrophotometers, flow cytometers, which may be hydrodynamically focused, imaging systems, imaging flow cytometers, and plate-based imaging systems. Nonlimiting examples of systems useful with the present methods include the Guava® EasyCyte™, the Guava® EasyCyte™ Mini, the Guava® PCA™, the Guava® PCA™-96, the Guava® EasyCyte™ Plus, FACS™ Aria, FACS™ Canto, Beckman Coulter Quanta™, Amnis ImageStream™, Molecular Devices ImageXpress™ apparatuses, and similar devices. Other apparatuses, including plate loading, plate washing, plate rocking, and similar devices useful for handling any assay components may be used.

### Fluorescence Resonance Energy Transfer Dye Pairs

The broad excitation and emission peaks of the fluorescent dyes provided by this disclosure enable good energy transfer between the dyes and enable them to be used in fluorescence resonance energy transfer (FRET) assays. FRET between the dye pairs of this disclosure allows the construction of a series of probes which can be utilized in flow cytometry and other imaging systems such as microscopy, fluorometers, spectrophotometers, and high content imaging etc. The FRET probes provided by this disclosure can also be used in Western blots followed by imaging, or in microarray based instruments for DNA or other biomolecule detection. The application of FRET between the dye pairs of this disclosure provides a new method of making tandem antibody/biomolecular probes.

In some embodiments, a FRET dye pair is provided which comprises a first fluorescent compound coupled to a first biomolecular segment and a second fluorescent compound coupled to a second biomolecular segment. The first fluorescent compound has a first excitation spectrum and a first emission spectrum. The second fluorescent compound has a second excitation spectrum and a second emission spectrum. The first emission spectrum of the first compound at least partially overlaps the second excitation spectrum of the second fluorescent compound. The first and second biomolecular segments can be on a same biomolecule. Alternatively, the first biomolecular segment is on a first biomolecule and the second biomolecular segment is on a second biomolecule different from the first biomolecule. The biomolecules can be any species that are produced by or obtained from a living organism, including cell or bacterial cultures. Exemplary biomolecules include proteins, peptides, polynucleotides, polysaccharides, antibodies, triglycerides, lipoproteins, and lectins. In some embodiments, the first and second biomolecules may comprise protein-protein, protein-oligosaccharide, oligosaccharide-oligosaccharide, protein-ligand.

One or both of the first and second fluorescent compounds may have the general formula I:

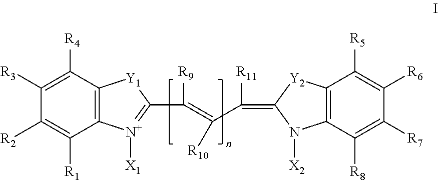

where:

R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;

R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;

Y₁ and Y₂ are independently selected from the group consisting of O, N, S, and —CR′R″— where R′ and R″ are independently H or C₁-C₁₈ alkyl;

X₁ and X₂ are independently selected from the group consisting of optionally substituted alkyl, optionally substituted heteroalkyl, and optionally substituted alkylaryl, wherein at least one of X₁ and X₂ is substituted alkylaryl comprising on the aryl component a substituted alkyl or heteroalkyl comprising a carboxylic acid substituent; and

n is 1, 2, or 3.

In some preferred embodiments, one or both of the first and second fluorescent compounds may have the general formula I where X₁ represents a group having the formula II:

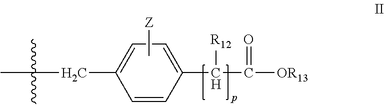

where Z is selected from the group consisting of H, SO₃H, optionally substituted alkyl, and optionally substituted phenyl; p is a number from 1 to 18; R₁₂ is H or C₁-C₁₈ alkyl, and R₁₃ is selected from the group consisting of the formulas III-a, III-b, III-c, III-d, and III-e:

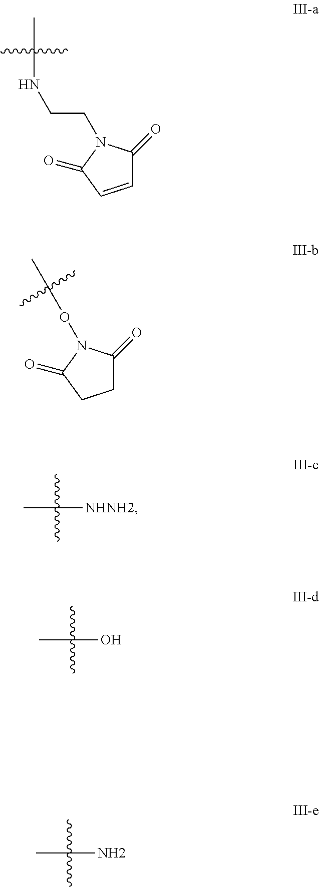

X₂ is the same as X₁, or a group of the formula IV below:

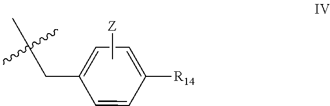

where R₁₄ is an optionally substituted alkyl or optionally substituted phenyl group, and Z is selected from the group consisting of H, SO₃H, optionally substituted alkyl, and optionally substituted phenyl.

In some preferred embodiments one or both of the first and second fluorescent compounds have the general formula I where X₁ is a group of the formula II-a:

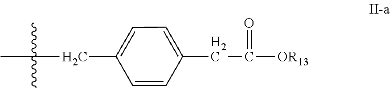

where R₁₃ is selected from the group consisting of the formulas III-a, III-b, III-c, III-d, and III-e shown above, and

X₂ is the same as X₁, or a group of

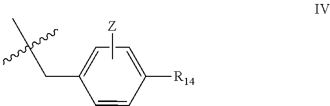

where R₁₄ is an optionally substituted alkyl or optionally substituted phenyl group, and Z is selected from the group consisting of H, SO₃H, optionally substituted alkyl, and optionally substituted phenyl.

In some preferred embodiments one or both of the first and second fluorescent compounds have the general formula I where one or both of X₁ and X₂ are a group of the formula II-a, n is 1, 2, or 3, R₃ and R₆ are independently H or SO₃H, and R₁-R₂ and R₆-R₁₁ are independently H.

In some preferred embodiments one or both of the first and second fluorescent compounds have the general formula I where n is 1, 2 or 3, R₃ and R₄, and R₅ and R₆ taken together respectively form a 6-membered ring optionally substituted by SO₃H or a derivative thereof, and R₁-R₂ and R₇-R₁₁ are independently H.

In some preferred embodiments one or both of the first and second fluorescent compounds have the structures provided in Table 1.

By way of example, an exemplary FRET dye pair comprises a first fluorescent compound having the formula N-1 or N-2 and a second fluorescent compound having the formula N-5 or N-6:

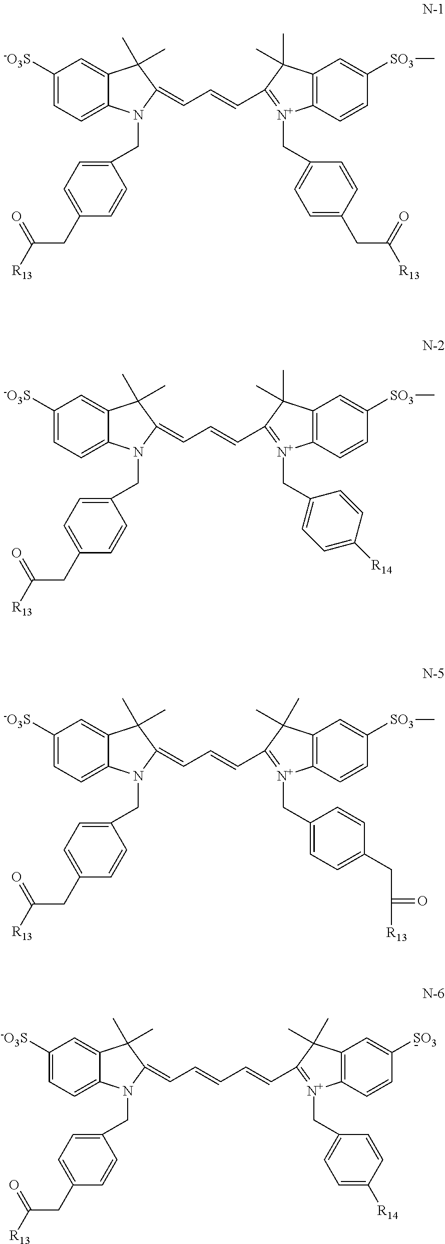

where R₁₃ is selected from the group consisting of the formulas III-a, III-b, III-c, III-d, and III-e, and R₁₄ is an optionally substituted alkyl or optionally substituted phenyl group.

The absorption spectrum of N-1 is shown in FIG. 18. The NHS esters of N-1 or N-2 (where R₁₃ is III-1D) are highly soluble and have been found to give rise to fluorescent conjugates that are excitable by light with a wavelength of about 532 nm and 488 nm and emit at about 577 nm. Thus, N-1 or N-2 is excitable by both green lasers (about 532 nm) and blue lasers (about 488 nm) and is detectable in the yellow channel (about 580 nm) of most flow cytometers. N-1 or N-2 and a FRET dye pair containing N-1 or N-2 can be used in both blue and green laser based instruments.

The absorption and emission spectrum of N-5 is also shown in FIG. 18. The NHS esters of N-5 or N-6 (where R₁₃ is III-1D) are highly soluble and have been found to give rise to fluorescent conjugates that are excitable by light with a wavelength of about 638 nm and emit at about 676 nm. Thus, N-5 or N-6 is excitable by red lasers (about 638 nm) and emits in the red channel (about 676 nm) of most flow cytometers. It is not detectable when only blue laser based excitation is employed.

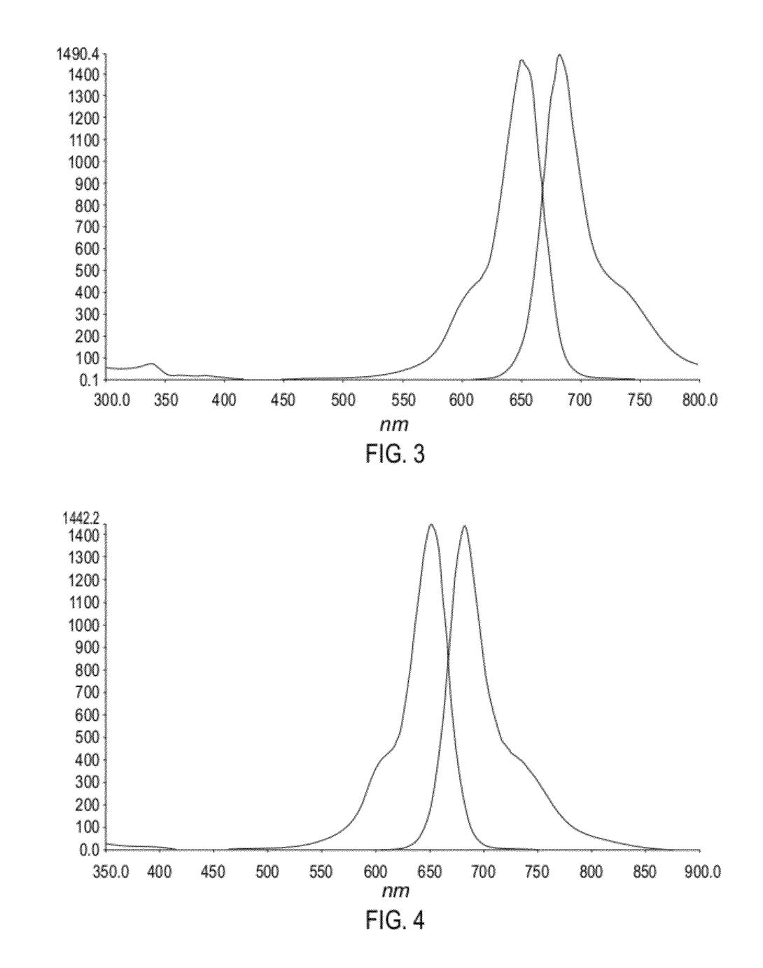

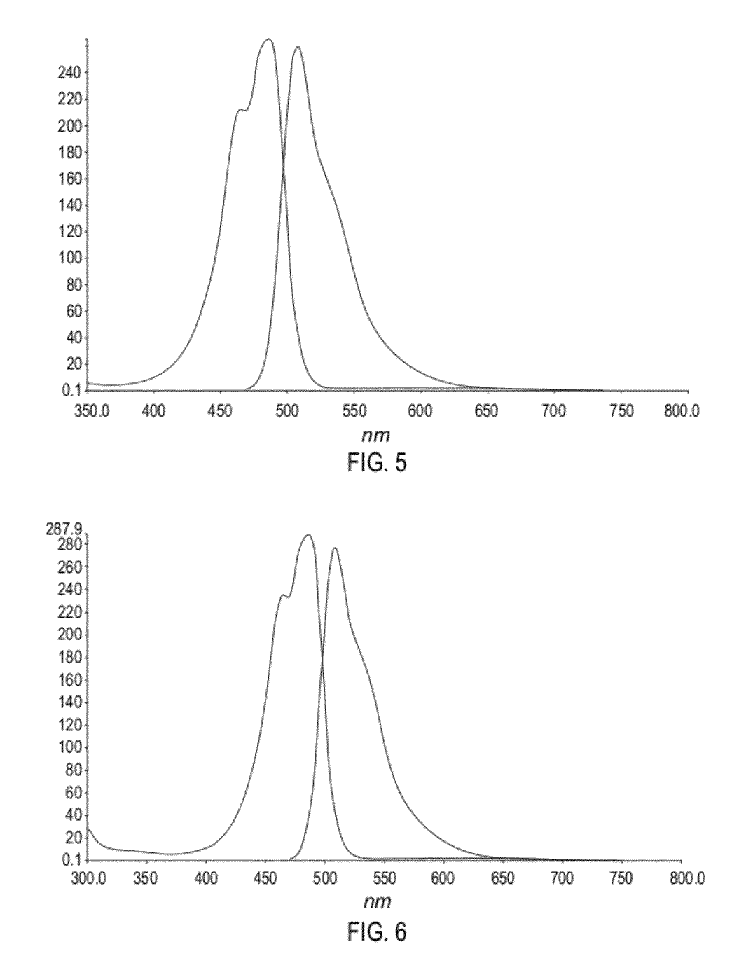

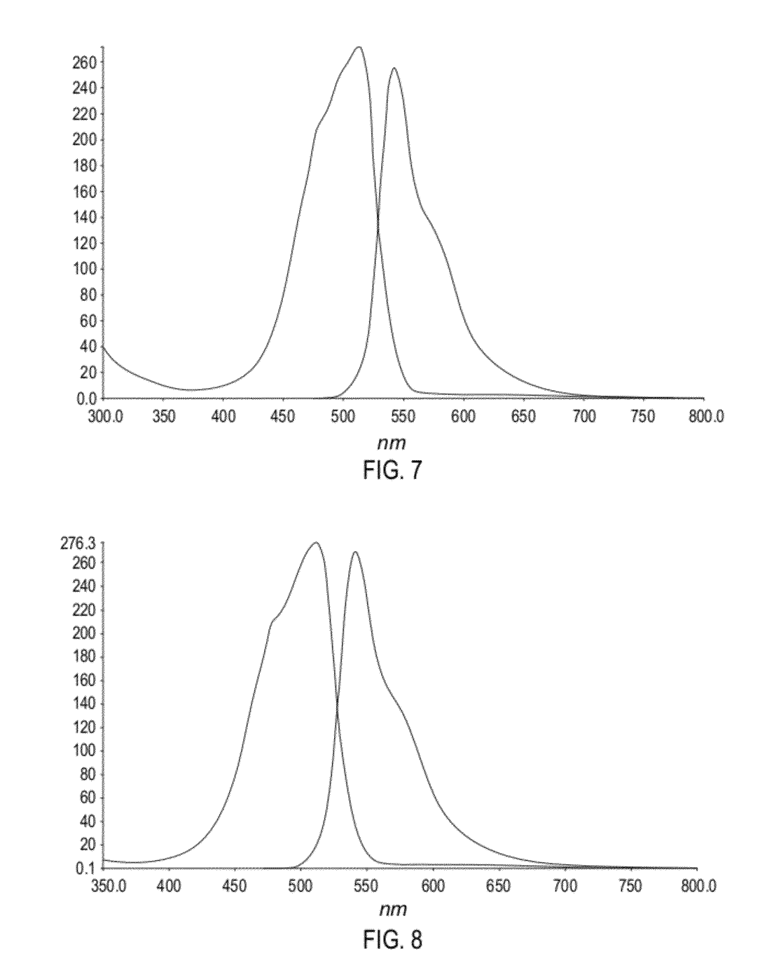

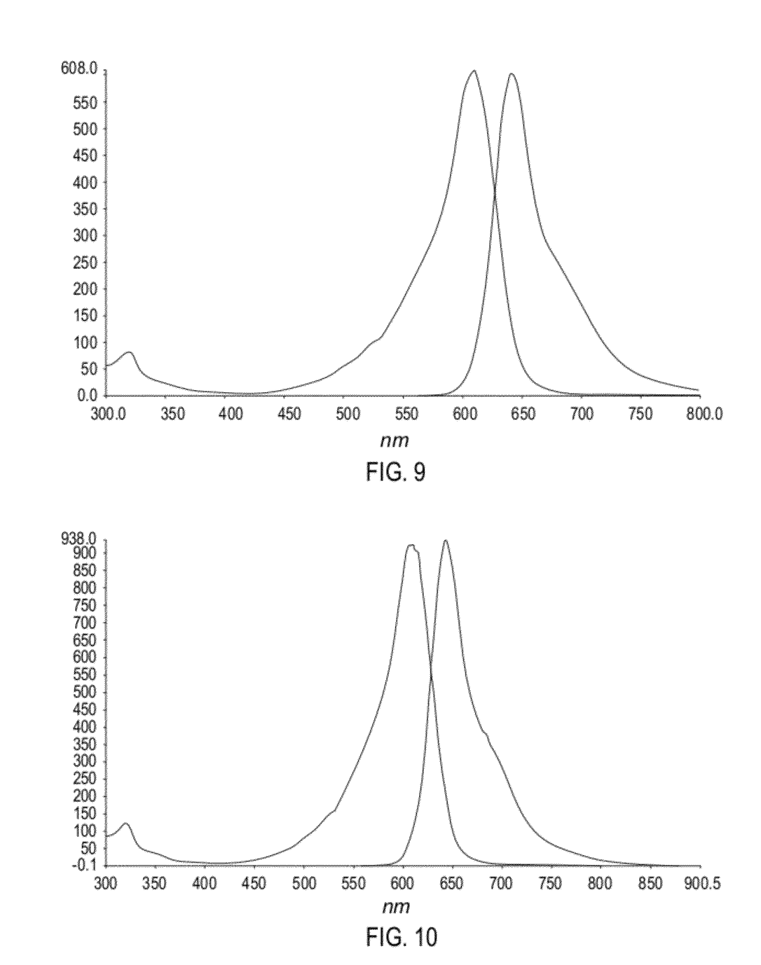

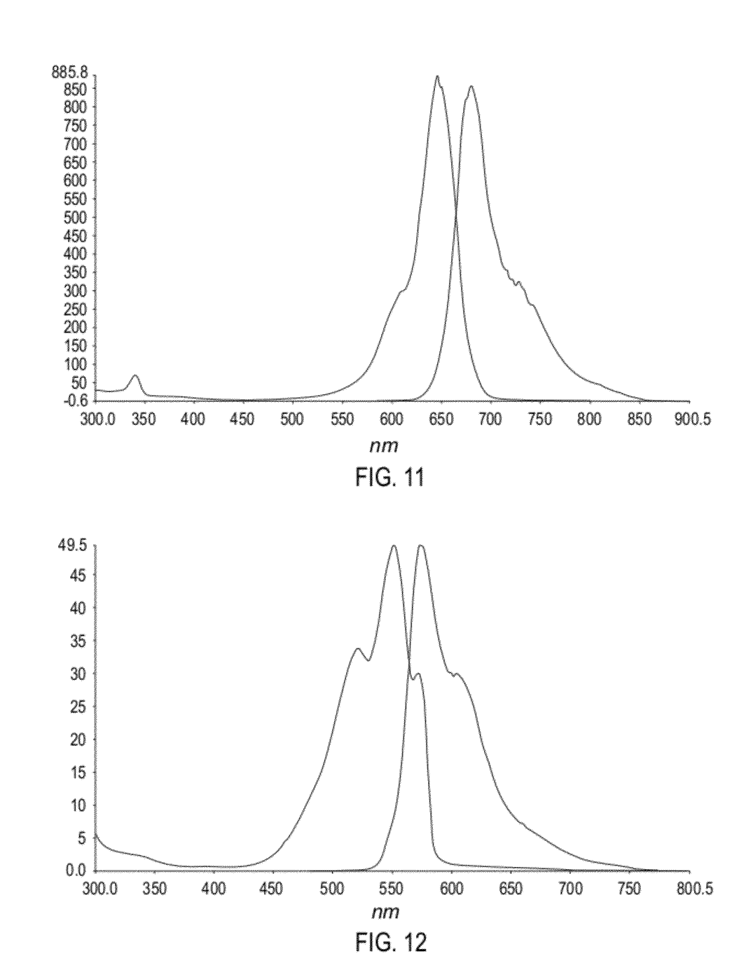

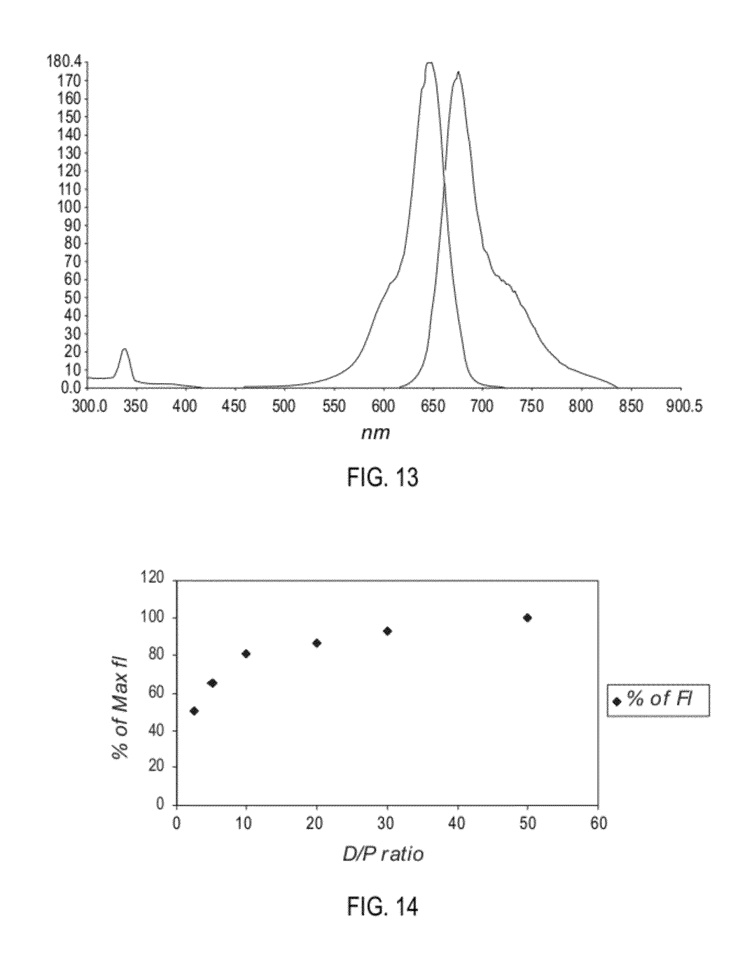

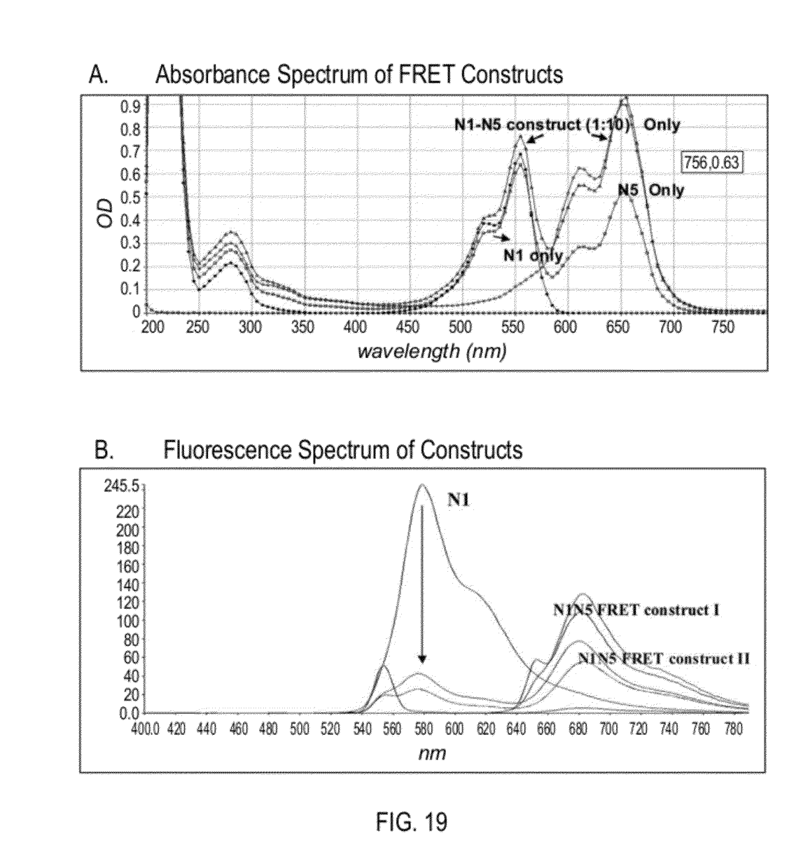

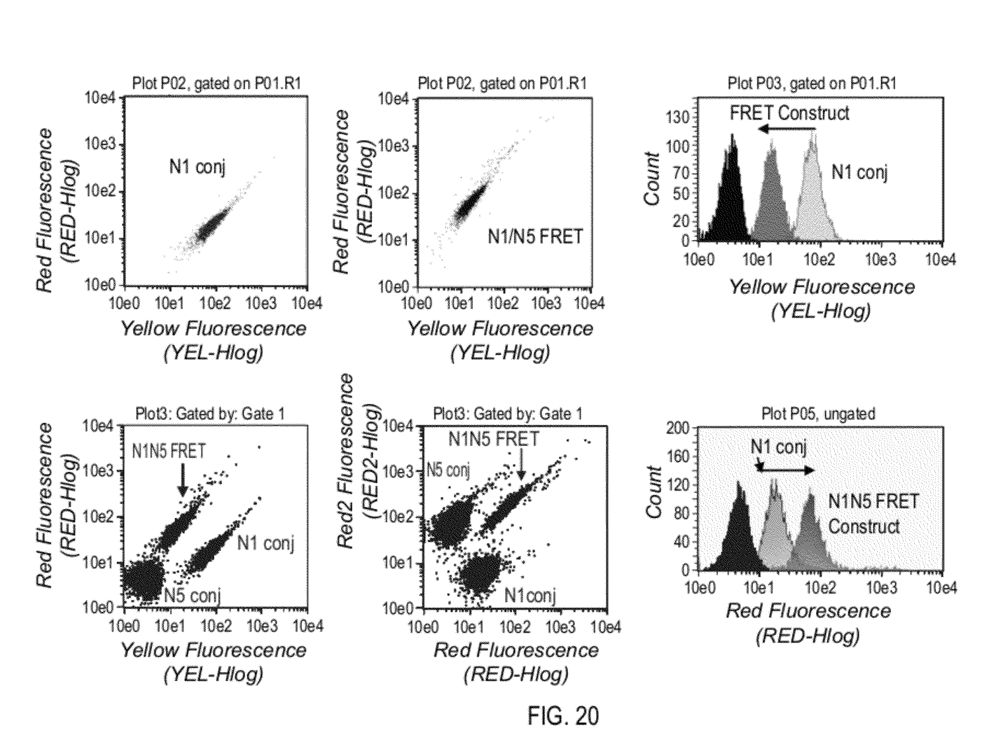

FIG. 19 shows absorption spectra of FRET constructs between N-1 and N-5. FIG. 20 shows fluorescence spectra of antibody-N-1 alone and FRET construct of antibody-N-1/N-5. Data in FIG. 3 clearly demonstrates decrease in fluorescence at 555 nm from N-1 in the FRET construct and increase in fluorescence at ˜676 nm due to FRET interactions. The lower panel is a fluorescence spectrum of a FRET construct when excited at 555 nm.

In another exemplary embodiment, a FRET dye pair comprises a first fluorescent compound having the formula N-5 or N-6 and a second fluorescent compound having the formula N-9 or N-10:

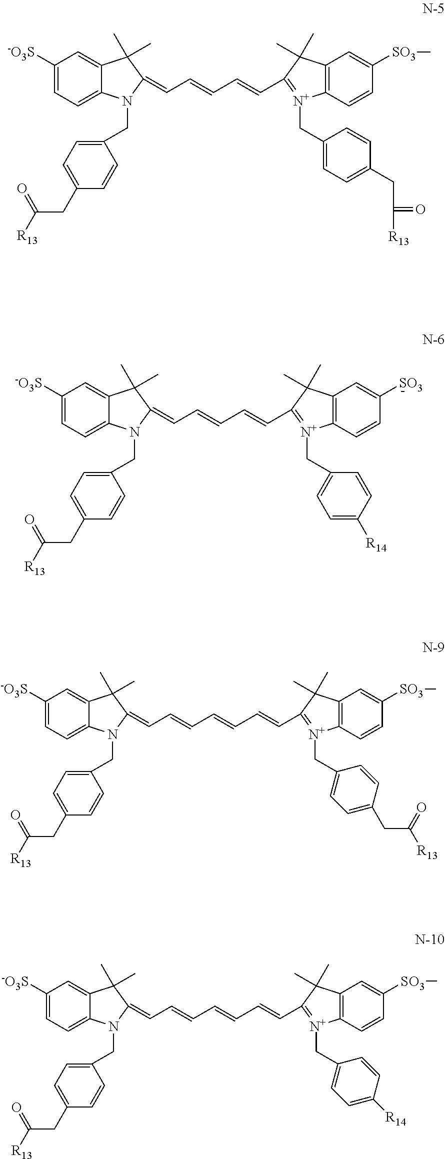

where R₁₃ is selected from the group consisting of the formulas III-a, III-b, III-c, III-d, and III-e, and R₁₄ is an optionally substituted alkyl or optionally substituted phenyl group.

Numerous other FRET dye pair combinations are possible where one or both of the first and second fluorescent compounds have the general formula I. In choosing the first and second fluorescent compounds, the emission spectrum of the first compound should at least partially overlap or preferably substantially overlap the excitation spectrum of the second fluorescent compound. The distance between the dipoles of the first and second fluorescent compounds are generally within about 2-8 nm (Foerster distance). In FRET when a donor dye and an acceptor dye are brought sufficiently close to each other a change in spectral response will take place. No change in spectral response indicates that there is absence of binding as donor fluorophore and acceptor fluorophores fluoresce normally.

In some embodiments, provided is a novel tandem probe which comprises a probe capable of binding to a binding partner, a first fluorescent compound coupled to the probe, and a second fluorescent compound coupled to the probe. The first fluorescent compound has a first excitation spectrum and a first emission spectrum. The second fluorescent compound has a second excitation spectrum and a second emission spectrum. The first emission spectrum of the first compound at least partially overlaps the second excitation spectrum of the second fluorescent compound. The probes may comprise a polynucleotide having a nucleic acid sequence which can bind to a corresponding binding partner. The polynucleotide regions of the probes may include DNA, and/or RNA, and/or synthetic nucleotide analogs. Binding partners can be any targets or analytes including such as cells, cell fragments, and cell surface molecules, for example immune system molecules, receptors, and markers indicative of specific cell populations or subpopulations.

One or both of the first and second fluorescent compounds may have the general formula I or an isomer, ester, amide, acid halide, acid anhydride, and/or salt thereof, or a mixture of any thereof:

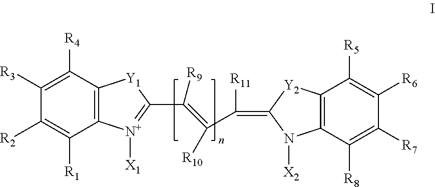

where:

R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;

R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;

Y₁ and Y₂ are independently selected from the group consisting of O, N, S, and —CR′R″— where R′ and R″ are independently H or C₁-C₁₈ alkyl;

X₁ and X₂ are independently selected from the group consisting of optionally substituted alkyl, optionally substituted heteroalkyl, and optionally substituted alkylaryl, wherein at least one of X₁ and X₂ is substituted alkylaryl comprising on the aryl component a substituted alkyl or heteroalkyl comprising a carboxylic acid substituent; and

n is 1, 2, or 3.

A method of making the new tandem probe is also provided. The method involves incubating a probe such as an antibody with a FRET dye pair at selected ratios. For example, a probe such as a non-fluorescent antibody may be incubated with a first fluorescent compound and a second fluorescent compound at a selected ratio, wherein the first fluorescent compound has a first excitation spectrum and a first emission spectrum, the second fluorescent compound has a second excitation spectrum and a second emission spectrum, and the first emission spectrum of the first compound at least partially overlaps the second excitation spectrum of the second fluorescent compound. One or both of the first and second fluorescent compounds may have the general formula I as described in greater detail above. The method allows for simplified synthesis of long stoke-shift tandem probes for flow cytometry. Antibody probes created using FRET dye pairs of this disclosure can also be used as sensors of environments that affect protein folding and hence the FRET of the probes. Conventional methods of making tandem probes such as PE-Cy5 are extremely complex, involve multiple steps, and have very low yields.

The energy transfer capability of the fluorescent compounds of this disclosure also allows transfer of energy to quenchers such as QSY-7, BHQ-2 etc., and creates substrates attached to the fluorescent compounds and quenchers. Within the close conformation the intensity of fluorescence emitted by the fluorescent compound is reduced or quenched due to the FRET energy transfer to the quencher. When the FRET to the quencher is disturbed e.g. by protease cleavage the biomolecule coupled with the fluorescent compound will become fluorescent and allow for detection. Therefore, in some embodiments, a conjugate is provided which comprises a fluorescent compound coupled to a first molecular segment and a non-fluorescent compound or a quencher coupled to a second molecular segment, where the fluorescent compound has an excitation spectrum and an emission spectrum, and the quencher absorbs energy with a spectrum that substantially overlaps the emission spectrum of the fluorescent compound.

In some embodiments, a method of detecting the proximity of one molecular segment to another molecular segment is provided. According to this method, a first fluorescent compound is coupled to a first molecular segment, and a second fluorescent compound is coupled to a second molecular segment. The first fluorescent compound has a first excitation spectrum and a first emission spectrum, the second fluorescent compound has a second excitation spectrum and a second emission spectrum, and the second excitation spectrum of the second fluorescent compound at least partially overlaps the first emission spectrum of the first compound. The first fluorescent compound is caused to be excited by illumination with an excitation beam having a spectrum that is at least partially overlaps the first excitation spectrum. The presence or absence of fluorescence that is characteristic of the second emission spectrum is detected. The proximity of the first molecular segment to the second molecular segment can be determined based on the presence or absence of the fluorescence that is characteristic of the second emission spectrum. If the first and second compounds are close to each other within the Foerster distance, a change in spectral response will take place.

### Cyanine Based Amine Reactive Viability Dyes

The cyanine compounds having the general formula I have good water solubility, show brightness and photo stability, and exhibit low non-specific binding, making them highly suitable for cellular viability measurements as amine reactive viability dyes.

The cyanine-based amine reactive viability dyes provided by this disclosure may be used to measure the integrity of cell membranes and the percentage or proportion of intact cells in a sample containing both intact cells and dead or damaged cells. The measurement is based on the principle that an intact cell has fewer exposed proteins thus fewer amino groups on the cell surface. If a cell membrane is compromised or damaged, a larger number of intracellular amino groups are exposed and the cell depicts a high level of staining with amine reactive fluorescent dyes.

In some embodiments, a method of determining the integrity of cell membranes is provided in which cells in a sample are incubated with a fluorescent cyanine compound having the general formula I. The cyanine compound is coupled to the cells and caused to emit fluorescence by e.g. directing an excitation beam to the sample. The intensity of the fluorescence emitted by the cyanine compound can be detected and compared with a predetermined value. The integrity of the cell membranes can be determined based on the comparison.

The fluorescence can be detected as described above with a variety of detection systems including flow cytometry, microscopy, microfluidic imaging, fluorometry, fluorescence and absorbance readers etc. The predetermined value of intensity can be provided by e.g. measuring live cells that are known to be intact. If the comparison shows that the detected intensity value is the same as the predetermined value then the cells can be determined as live or having intact membranes. If the comparison shows that the detected intensity value is substantially greater than the predetermined value then the cells can be determined as dead or having damaged membranes.

In some embodiments, a method is provided to determine the percentage or proportion of intact cells in a sample containing both intact cells and dead or damaged cells. Cells may be subject to death due to development or disease or caused by treatment with external agents or due to various other environmental reasons. According to the provided method, a sample containing cells with intact membranes and cells with damaged membranes is incubated with a fluorescent cyanine compound having the general formula I. The cyanine compound is coupled to cells with intact membranes and cells with damaged membranes respectively, and caused to emit fluorescence. The fluorescence emitted by the cyanine compound is detected and the difference of the intensity of the fluorescence ascertained. The proportion of the cells with intact membranes in the sample can be determined based on the difference of the intensity of the fluorescence.

The cyanine-based amine reactive viability dyes provided by this disclosure may have the general formula I:

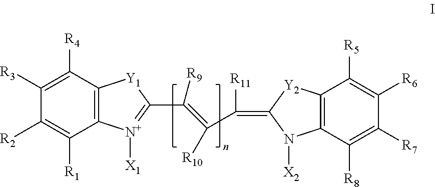

where:

R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;

R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;

Y₁ and Y₂ are independently selected from the group consisting of O, N, S, and —CR′R″— where R′ and R″ are independently H or C₁-C₁₈ alkyl;

X₁ and X₂ are independently selected from the group consisting of optionally substituted alkyl, optionally substituted heteroalkyl, and optionally substituted alkylaryl, wherein at least one of X₁ and X₂ is substituted alkylaryl comprising on the aryl component a substituted alkyl or heteroalkyl comprising a carboxylic acid substituent; and

n is 1, 2, or 3.

By way of example, compounds that are particularly suitable as amine reactive viability dyes have the following formulas:

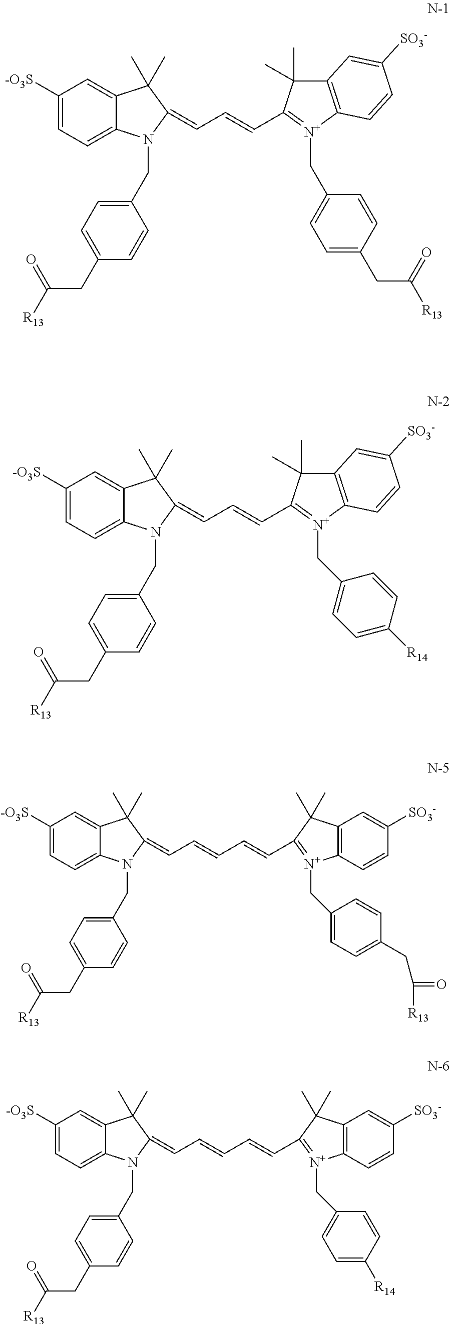

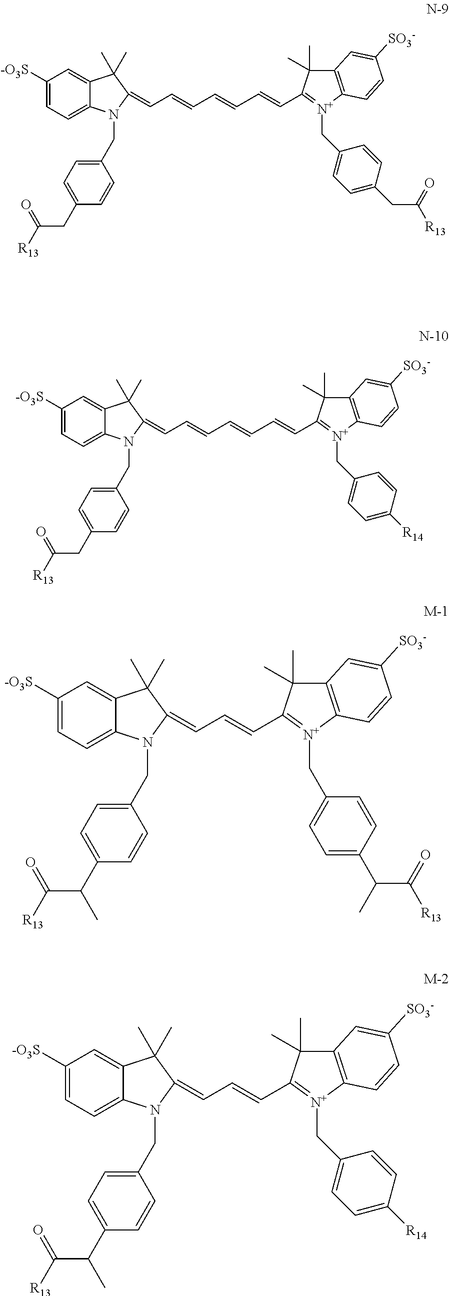

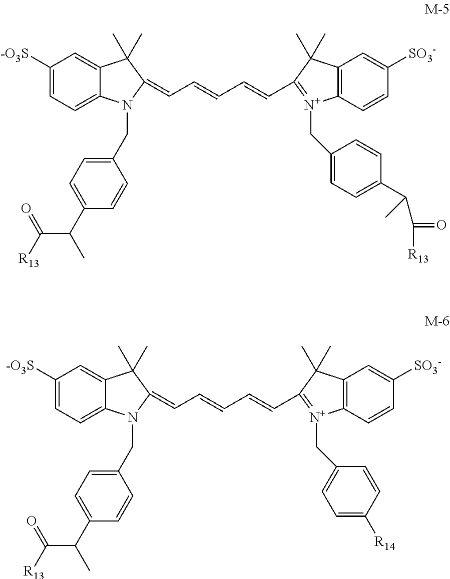

where R₁₃ is selected from the group consisting of the formulas III-a, III-b, III-c, III-d, and III-e:

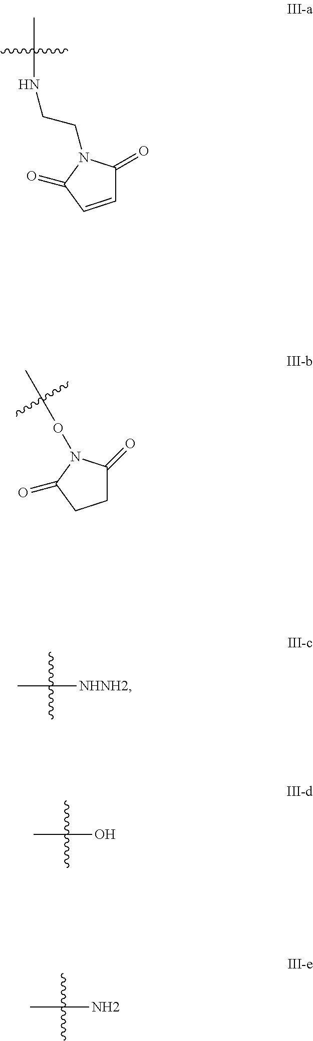

R₁₄ is an optionally substituted alkyl or optionally substituted phenyl group.

It should be noted that the above exemplary compounds are provided for illustration purpose only. Any suitable compounds having the general formula I, including those listed Tables 1-5, can be used as amine reactive viability dyes.

Advantageously, the viability dyes provided by this disclosure can be used in cellular viability measurement where wash steps are required. The dyes can also be used with no wash steps. The dyes may be used where fixation of biological samples or permeabilization of cells is required, or fixation and permeabilization of samples are required for an assay. The intensity differences in fluorescence from the intact and damaged cells are preserved following the fixation and/or permeabilization of the sample. In certain assays, permeabilization of cells may be needed to make cells membranes permeant to allow probes, dyes, or other chemicals passing through for binding an intracellular analyte. Permeabilization of cells can be done physically such as in microinjection, by electrical breakdown, or by mechanical manipulation. Alternatively, cell membranes can be permeabilized by treatment with fixatives or chemical agents. Permeabilization and fixation of cell samples are well known in the art.

Another advantage of the cyanine-based amine reactive viability dyes provided by this disclosure is that they can be excited by the sources that are commonly found in flow cytometry or other imaging systems. Their emission can be detected in the detection windows commonly found in these instruments. For example, Compound N-1 or N-2 as a viability dye can be used with flow cytometers equipped with blue (488 nm) and/or green (˜532-555) light sources. Compound N-5 or N-6 as a viability dye can be used with flow cytometers equipped with a red (˜638 nm) light source.

The viability dyes provided by this disclosure may be used in combination with other cell typing or antibody markers in other colors. The probes may be labeled with other fluorescent dyes to enable multiplexed detection of the cells.

In an exemplary experiment protocol, a sample of cells can be prepared in a single cell suspension. A working solution can be prepared from a dye solution comprising a dye provided by this disclosure. The sample and working solutions are mixed and incubation of the dye with cells can be carried out at the room temperature. After incubation, excess dyes that are not coupled to cells may be removed. The cells can be washed and re-suspended for measurement. If fixation and/or permeabilization are required for an assay, the cells may be re-suspended in a permeabilization reagent, and/or incubated on ice. The cell pellets can be then washed and re-suspended for measurement.

### EXAMPLES

The following examples are set forth so as to provide those of ordinary skill in the art with a complete description of how to make and use the present invention, and are not intended to limit the scope of what is regarded as the invention. Efforts have been made to ensure accuracy with respect to numbers used (e.g., amounts, temperature, etc.) but some experimental error and deviation should be accounted for. In some instances, although the reactions are shown as producing a particular form of the compound, the compound may be protonated at one or more of the acidic positions, as one or more salts, or as mixtures of any thereof. Unless otherwise indicated, parts are parts by weight, temperature is degree centigrade and pressure is at or near atmospheric, and all materials are commercially available.

### Example 1

### Preparation of 2,3,3-trimethyl-5-sulfoindolium, Potassium Salt (Compound No. 1)

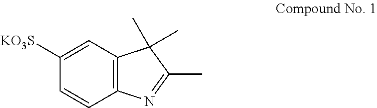

A mixture of 4-Hydrazinobenzenesulfonic acid (20.0 g, 106.0 mmol) and 3-Methyl-2-butanone (36.0 mL, 336.0 mmol) in glacial acetic acid (50 mL) was heated to reflux for 3 h. During this period, the reaction became homogenous and turned into dark red. The mixture was cooled to room temperature and the dark red solid was collected by filtration and dried under vacuum. The resulting solid was dissolved in methanol (200 mL) and a solution of KOH/IPA (2M) was added until basic. The yellow solid was filtered off and dried under vacuum overnight to furnish compound No. 1 (23.6 g, 80%, (M+H¹=240.1).

### Example 2

### Preparation of Compound No. 2

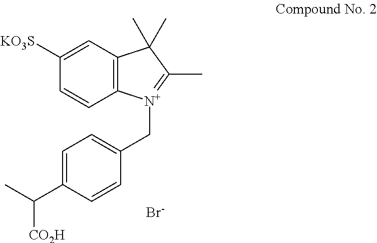

A mixture of compound No. 1 (1.0 g, 3.6 mmol) and 2-[4-(bromomethyl)phenyl]propanoic (0.88 g, 3.6 mmol) in 1,2-dichlorobenzene (20 mL) was heated to 110° C. for over night. The solvent was decanted. To the purple residue was added isopropyl alcohol (IPA) and stirred. The purple solid was filtered off and dried to give compound No. 2 (1.35 g, 72%, M+H¹=402.2).

### Example 3

### Preparation of Compound No. 3

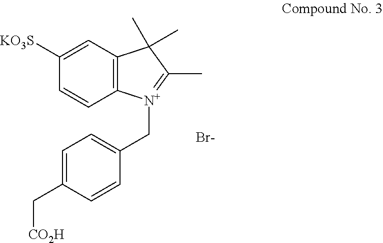

A mixture of compound No. 1 (5.0 g, 18.0 mmol) and 4-(bromomethyl)phenyl acetic acid (5.0 g, 21.83 mmol) in 1,2-dichlorobenzene (10.0 mL) was heated to 135° C. for 2 h. The solvent was decanted and the solid was dried to give compound No. 3 as a dark pink solid (6.92 g, 75.8%, M+H¹=388.1).

### Example 4

### Preparation of Compound No. 4 (Structure M-1 in Table 2)

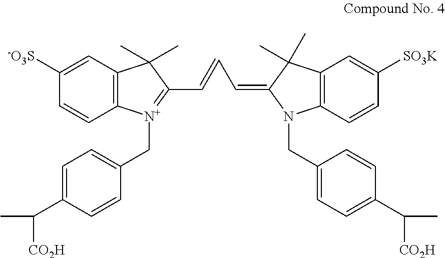

A mixture of compound No. 2 (0.120 g, 0.23 mmol) and triethyl orthoformate (0.20 mL, 1.2 mmol) in pyridine (3.0 mL) was heated to reflux for 1 h. The mixture was concentrated to give a dark pink residue. The purification of this crude product by reversed phase HPLC (Acetonitrile/water, 0.1% TFA) furnished compound No. 4 as a pink solid (0.130 g, 66%, M+H¹=813.1, Ex=560 nm, Em=579 nm in Methanol).

### Example 5

### Preparation of Compound No. 5 (Structure M-1 in Table 2)

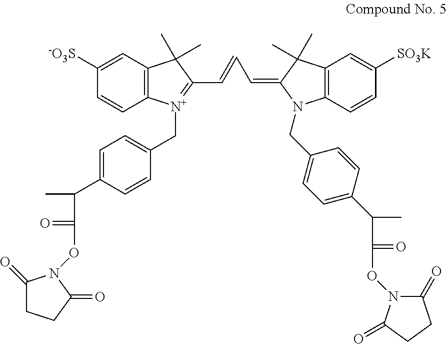

A mixture of compound No. 4 (0.04 g, 0.06 mmol), and N,N′-disuccinimidyl carbonate (0.10 g, 0.39 mmol), in a mixture of pyridine (0.1 mL) and DMF (2 mL) was heated to 55° C. for 2 h. The mixture was washed with ether, dichloromethane then dried in speed vac. overnight to give compound No. 5 as a pink solid (0.045 g, 77.5%, M+H¹=1007.2, Ex=560 nm, Em=579 nm in Methanol).

### Example 6

### Preparation of Compound No. 6 (Structure M-5 in Table 2)

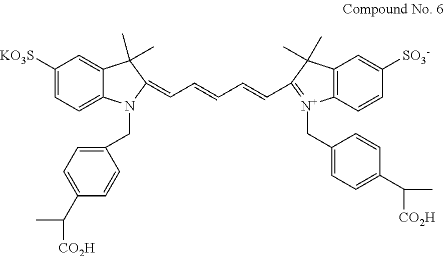

A mixture of compound No. 2 (0.250 g, 0.48 mmol), 1,1,3,3-tetramethoxypropane (0.340 mL, mmol), Acetic acid (0.120 mL, mmol), and acetic anhydride (0.160 mL, 0.98 mmol) in 1-methyl-2-pyrrolidnone (2 mL) was heated to 50° C. for over night. The mixture was concentrated under reduced pressure to give a dark blue residue. The purification of this crude product by reversed phase HPLC (Acetonitrile/water, 0.1% TFA) provided compound No. 6 as a dark blue solid (0.165 g, 39.2%, M+H¹=839.8, Ex=650 nm, Em=682 nm in Methanol).

### Example 7

### Preparation of Compound No. 7 (Structure M-5 in Table 2)

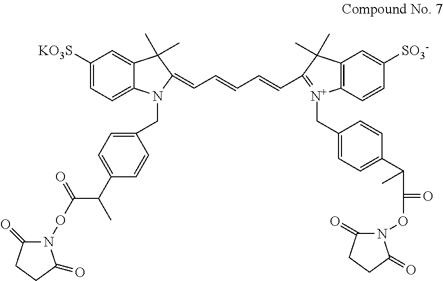

A mixture of compound No. 6 (0.030 g, 0.04 mmol), N,N′-disuccinimidyl carbonate (0.06 g, 0.74 mmol), and pyridine (0.10 mL) in DMF (2 mL) was stirred at 55° C. for over night. The mixture was washed with ether, dichloromethane and dried vacuum to furnish compound No. 7 as a dark blue solid (0.035 g, 95%, M+H¹=1007.2, Ex=650 nm, Em=682 nm in Methanol).

### Example 8

### Preparation of Compound No. 8

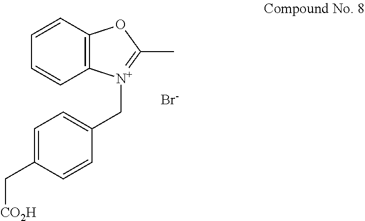

A mixture of 4-(bromomethyl)phenyl acetic acid (1.0 g, 4.37 mmol) and 2-methylbenzoxazole (1.0 mL, 8.42 mmol) was heated neat to 140° C. for 30 min. The melt was cooled to room temperature and was collected and dried to give compound No. 8 (1.25 g, 79%, M+H¹=282).

### Example 9

### Preparation of Compound No. 9 (Structure H-1 in Table 4)

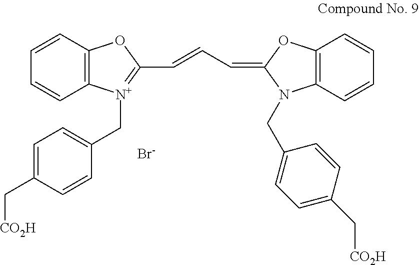

A mixture of compound No. 8 (1.10 g, 3.05 mmol) and triethyl orthoformate (0.35 mL, 2.1 mmol) in pyridine (8.0 mL) was heated to 120° C. for 3 h. The mixture was concentrated to give a dark pink residue. The purification of this crude product by reversed phase HPLC (Acetonitrile/water, 0.1% TFA) furnished compound No. 9 as a pink solid (0.120 g, 80.6%, M+H¹=574.3, Ex=489 nm, Em=507 nm in Methanol).

### Example 10

### Preparation of Compound No. 10 (Structure H-1 in Table 4)

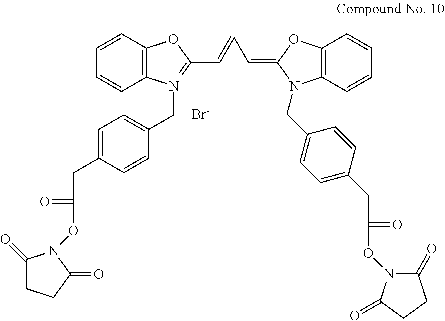

A mixture of compound No. 9 (0.045 g, 0.07 mmol) and N,N′-disuccinimidyl carbonate (0.12 g, 0.47 mmol) in pyridine (0.1 mL) and DMF (2 mL) was heated to 55° C. for 2 h. The mixture was washed with ether, dichloromethane and dried vacuum to furnish compound No. 10 as a pink solid (0.048 g, 85.7%, M+H¹=768.1, Ex=490 nm, Em=508 nm in Methanol).

### Example 11

### Preparation of Compound No. 11 (Structure H-17 in Table 4)

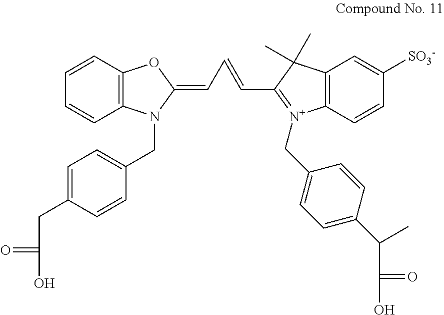

A mixture of compound No. 8 (0.180 g, 0.50 mmol) and N,N′-diphenylformamidine (0.11 g, 0.54 mmol) in acetic anhyride (3.0 mL) and acetic acid (3 mL) was heated to 100° C. for 1 h then was cooled to room temperature. To this mixture was added acetic anhydride (3 mL) and pyridine (3 mL) and was heated to 100° C. for 1 h. The mixture was concentrated to give the crude product. The purification of this crude product by reversed phase HPLC (Acetonitrile/water, 0.1% TFA) furnished compound No. 11 as a pink solid (0.25 g, 72.5%, M+H¹=693.2, Ex=510 nm, Em=541 nm in Methanol).

### Example 12

### Preparation of Compound No. 12 (Structure H-17 in Table 4)

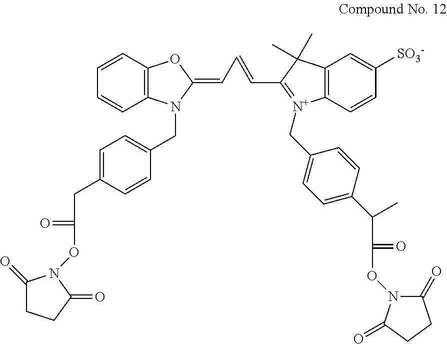

A mixture of compound No. 11 (0.05 g, 0.07 mmol) and N,N′-disuccinimidyl carbonate (0.10 g, 0.39 mmol) in pyridine (0.1 mL) and DMF (2 mL) was heated to 55° C. for 2 h. The mixture was washed with ether, dichloromethane and dried vacuum to furnish compound No. 12 as a pink solid (0.06 g, 85.9%, M+H¹=887.4, Ex=510 nm, Em=541 nm in Methanol).

### Example 13

### Preparation of Compound No. 13 (Structure H-5 in Table 4)

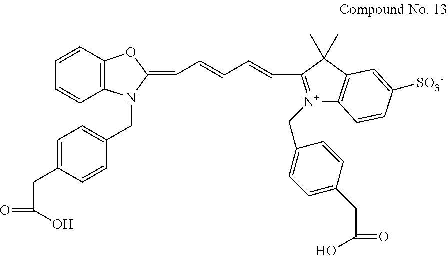

A mixture of compound No. 8 (0.36 g, 0.99 mmol) and malonaldehyde dianilide hydrochloride (0.29 g, 1.10 mmol) in acetic anhyride (6.0 mL) and acetic acid (6 mL) was heated to 115° C. for 1 h then was cooled to room temperature. To this mixture was added compound No. 3 (0.55 g, 1.1 mmol), acetic anhydride (6 mL), and pyridine (12 mL), and heated to 115° C. for 1 h. The mixture was concentrated to give the crude product. The purification of this crude product by reversed phase HPLC (Acetonitrile/water, 0.1% TFA) furnished compound No. 13 as a pinkish blue solid (0.415 g, 59.5%, M+H¹=899.2, Ex=609 nm, Em=641 nm in Methanol).

### Example 14

### Preparation of Compound No. 14 (Structure H-5 in Table 4)

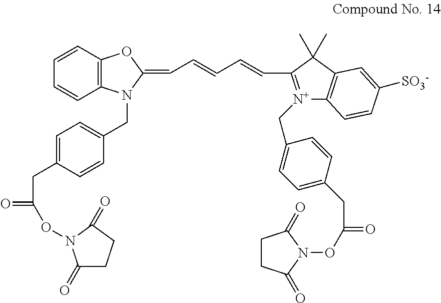

A mixture of compound No. 13 (0.025 g, 0.04 mmol), N,N′-dicyclohexylcarbodiimide (0.074 g, 0.36 mmol), and N-hydroxysuccinimide (0.083 g, 0.72 mmol) in DMF (2 mL) was stirred at room temperature overnight. The mixture was washed with ether, ethyl acetate then dried in speed vac overnight to furnish compound No. 14 as a pinkish blue solid (0.023 g, 71.9%, M+H¹=899.2, Ex=609 nm, Em=642 nm in Methanol).

### Example 15

### Preparation of Compound No. 15

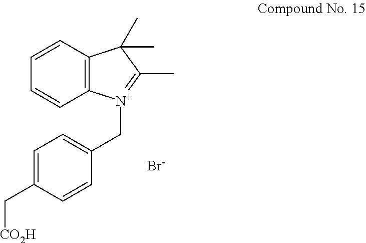

A mixture of 2,3,3-trimethylindolenine (1.3 mL, 8.10 mmol) and 4-(bromomethyl)phenyl acetic acid (1.0 g, 5.40 mmol) in 1,2-dichlorobenzene (10 mL) was heated to 140° C. for 3 h. The mixture was cooled to room temperature and was diluted with ether. The purple solid product was obtained by filtration and dried to give compound No. 15 (1.43 g, 84.3%, M+H¹=308.1).

### Example 16

### Preparation of Compound No. 16 (Structure N-10 in Table 1)

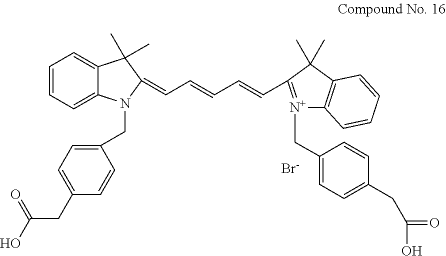

A mixture of compound No. 18 (0.50 g, 1.29 mmol), 1,1,3,3-tetramethoxypropane (0.32 mL, 1.93 mmol), acetic acid (0.150 mL, 2.58 mmol), and acetic anhydride (0.73 mL, 7.74 mmol) in 1-methyl-2-pyrrolidnone (2 mL) was heated to 50° C. for overnight. The mixture was concentrated under reduced pressure to give a dark blue residue. The purification of this crude product by reversed phase HPLC (Acetonitrile/water, 0.1% TFA) provided compound No. 16 as a dark blue solid (0.56 g, 59.4%, M+H¹=652.4, Ex=650 nm, Em=679 nm in Methanol).

### Example 17

### Preparation of Compound No. 17

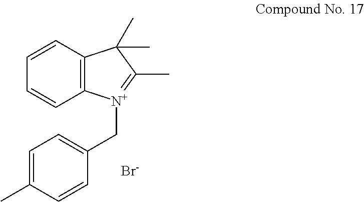

A mixture of 2,3,3-trimethylindolenine (1.3 mL, 8.10 mmol) and 4-methyl benzyl bromide (1.0 g, 5.40 mmol) in 1,2-dichlorobenzene (5 mL) was heated to 140° C. for 1 h. The mixture was cooled to room temperature and diluted with ether. The purple solid product was obtained by filtration and dried to give compound No. 17 (0.263 g, 76.5%, M+H¹=265.0).

### Example 18

### Preparation of Compound No. 18

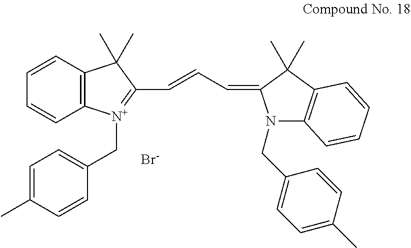

A mixture of compound No. 17 (0.25 g, 0.73 mmol) and triethyl orthoformate (0.60 mL, 3.63 mmol) in pyridine (4.0 mL) was heated to reflux for 2 h. The mixture was concentrated to give a dark pink residue. The purification of this crude product by reversed phase HPLC (Acetonitrile/water, 0.1% TFA) furnished compound No. 18 as a pink solid (0.35 g, 78.7%, M+H¹=538.2, Ex=551 nm, Em=574 nm in Methanol).

### Example 19

### Preparation of Compound No. 19

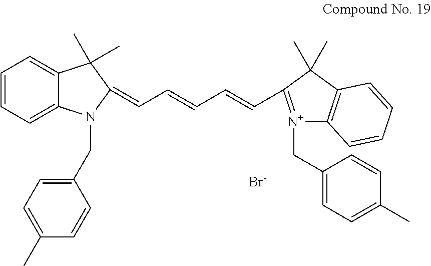

A mixture of compound No. 17 (0.390 g, 1.14 mmol), 1,1,3,3-tetramethoxypropane (0.280 mL, 1.7 mmol), acetic acid (0.130 mL, 2.26 mmol), and acetic anhydride (0.64 mL, 6.78 mmol) in 1-methyl-2-pyrrolidnone (2 mL) was heated to 50° C. for overnight. The mixture was concentrated under reduced pressure to give a dark blue residue. The purification of this crude product by reversed phase HPLC (Acetonitrile/water, 0.1% TFA) provided compound No. 19 as a dark blue solid (0.36 g, 49.6%, M+H¹=564.3, Ex=650 nm, Em=674 nm in Methanol).

### Example 20

### Use of Dyes in Biomolecular Conjugates

Synthesis of Conjugates: Goat-anti mouse antibodies (or equivalent antibodies) were labeled with NHS esters of fluorescent cyanine dyes in a 50 mM sodium carbonate buffer, pH8.0 by adding different D/P ratios. The mixture was incubated at room temperature. After 1.5-2 hrs the conjugates were removed and purified by gel filtration. Clear separation of conjugates from dyes was observed. Fractions containing conjugated protein were pooled together.

Absorbance Analysis: Absorbance analysis was performed on a Spectramax system and fluorescence analysis was performed on a Perkin Elmer fluorimeter.

Cellular Evaluation: Jurkat cells (50,000) were incubated with ˜1 ug of CD45 primary antibody or isotype control in a total vol of 20 uL for 20 min at RT. This was next diluted to 200 uL, centrifuged and supernatant removed. Cells were next bought up in 10 uL vol of buffer containing PBS, 0.08% azide and 1% BSA. Fluorescently labeled Secondary antibody (0.5-2 ug per test) was next added in a volume of 10 uL and the mix incubated for 20 min. The wells were next bought up to a volume of 200 uL, using buffer above, centrifuged. The pellet was next re-suspended in 200 uL of PBS, 0.08% azide, 1% BSA and samples were analyzed by flow cytometry. Flow cytometry was performed in a Guava EasyCyte 8HT system equipped with a blue and red laser and on a green PCA-96 system with a green laser.

FIG. 14 shows the impact of different Dye to Protein (D/P) ratios on the fluorescence of M1 antibody conjugates. The data shows that when treated with different D/P ratios there was no quenching or loss of fluorescence as more and more dyes wer added to the conjugate.

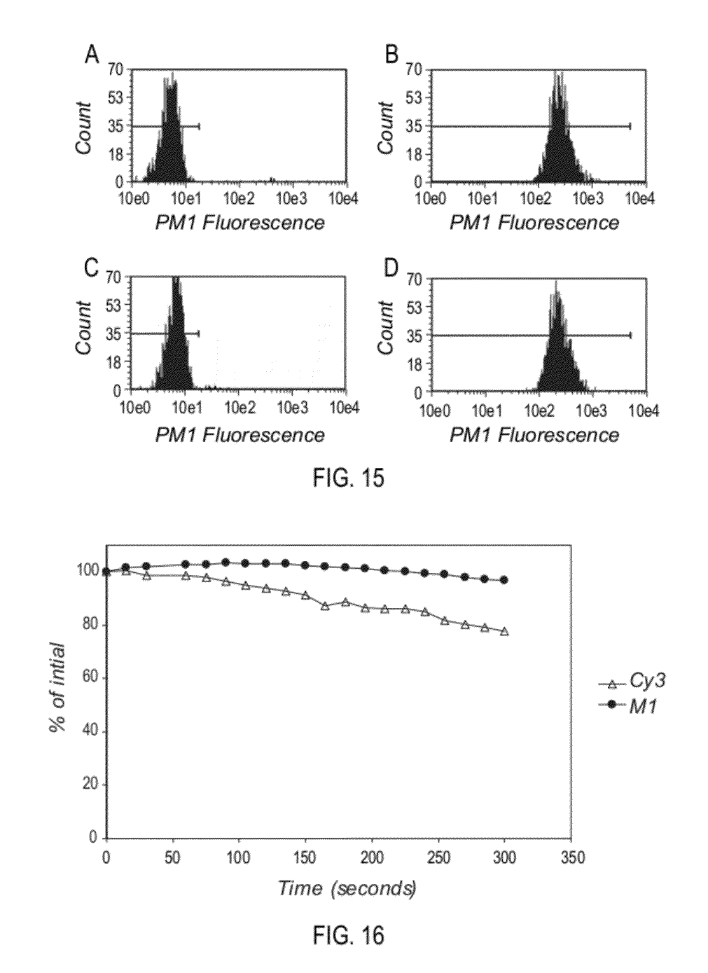

FIG. 15 shows the utility of M1 conjugates in cellular applications. Antibody conjugates of M1 and N1 were compared for use as secondary antibodies for cellular staining on Jurkat cells using isotype control (A and C) and CD45 primary antibodies (B and D). Superior S/N was observed for M1 conjugates (B) and N1 conjugates (D) when used as a secondary antibody and analyzed by flow cytometry. Both conjugates gave good separation and can be used for excitation with a green laser.

FIG. 16 shows the photobleaching characteristics of M1 antibody conjugates. Antibody conjugates of M1 and Cy3 were compared for use as secondary antibodies for Jurkat cells using CD45 primary antibodies. Stained cells were analyzed by fluorescent microcscopy. M1 conjugates demonstrated superior photostability compared to Cy3 conjugates for fluorescence visualization.

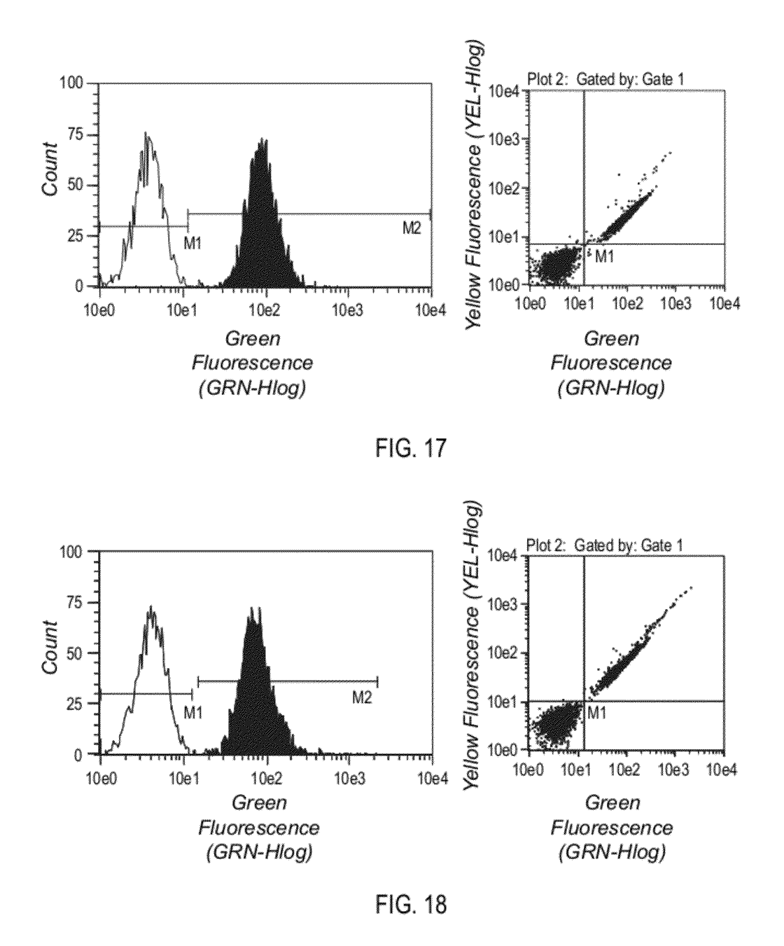

FIG. 17 illustrates an example where H-1 NHS was used to conjugate and create secondary labeled antibodies which can be excited by a blue laser (e.g. 488 nm). Detection of the antibody conjugated with the dye can be performed in the green channel of most optical instrumentation (˜510-530 nm). The antibody was used to probe Jurkat samples stained with unlabeled CD45 followed by secondary antibody and detected with flow cytometry. The conjugates demonstrate good separation and detection in the green channel of the flow cytometer.

FIG. 18 illustrates a further example where H-3 NHS was used to conjugate and create secondary labeled antibodies which can be excited by a blue laser (e.g. 488 nm). H-3 NHS was used to conjugate and create secondary labeled antibodies. The antibody was used to probe Jurkat samples stained with unlabeled CD45 followed by secondary antibody. The conjugates demonstrate good separation and detection in both the green and yellow channels of the flow cytometer.

### Example 21

### Synthesis of FRET Conjugates

Goat-anti mouse antibodies were labeled with both Compound N-1 and Compound N-5 in a buffer (sodium carbonate, 50 mM, pH 8.0) by adding N-1 followed immediately by the addition of N-5. The conjugate was incubated at the room temperature. After 1.5-2 hours the conjugates were removed and purified by gel filtration. Clear separation of conjugates from dyes was observed. Fractions containing conjugated protein were pooled together.

The absorbance analysis was performed on a Spectramax system from Molecular Devices and the fluorescence analysis was performed on a fluorometer from Perkin Elmer.

FIG. 19 shows the absorbance and fluorescence spectra of the FRET constructs. Panel A shows the absorbance spectrum when N1 alone was used to conjugate to the antibody (peak max ˜555 nm), N5 alone was used to conjugate to the antibody (peak max of 651 nm) and when both dyes were used to label the antibodies (2 peaks at ˜555 nm and ˜650 nm). The fluorescence data in Panel B shows that the resulting FRET construct can be excited at 555 nm and gives emission at both 576 nm and at 676 nm. Hence energy transfer has taken place between the dyes. The dyes thus demonstrate capability to energy transfer and for their use in energy transfer experiments as FRET pairs.

Cellular Evaluation: Jurkat cells (˜50,000) were incubated with ˜1 ug of CD45 primary antibody (or isotype control) in a total volume of 20 uL for 20 min at the room temperature. The mixture was next diluted to 200 uL and centrifuged. The supernatant was removed. Cells were next brought up in 10 uL vol of a buffer containing phosphate-buffered saline (PBS), 0.08% azide and 1% bovine serum albumin (BSA). Secondary antibody (0.5-2 ug per test) was next added in a volume of 10 uL and the mixture was incubated for 20 min. The wells were next brought up to a volume of 200 uL using the above buffer and centrifuged. The pellet was next re-suspended in 200 uL of PBS, 0.08% azide, and 1% BSA, and the samples were analyzed by flow cytometry. Flow cytometry was performed on Guava EasyCyte 8HT system equipped with a blue and a red laser and on a PCA-96 system equipped with a green laser.

### Example 22

### Detection of FRET by Flow Cytometry

FIG. 20 illustrates detection of CD45 and isotype on Jurkat cells using goat anti-mouse antibody constructs. Jurkat cells were stained with CD45 primary antibody followed by the following secondary antibodies labeled by (1) Compound N-1 alone, (2) Compound N-5 alone, and (3) both N-1 and N-5. FRET was clearly detectable as shown in FIG. 20 and was distinguishable in cases where only one fluorophore was present. The characteristics of fluorescence demonstrate that N1 and N5 can energy transfer to each other and be used in experiments where FRET is required as is evident from an increase in red fluorescene and decrease in yellow fluorescence.

### Example 23

### Antibody-FRET Pair as a Tandem Probe

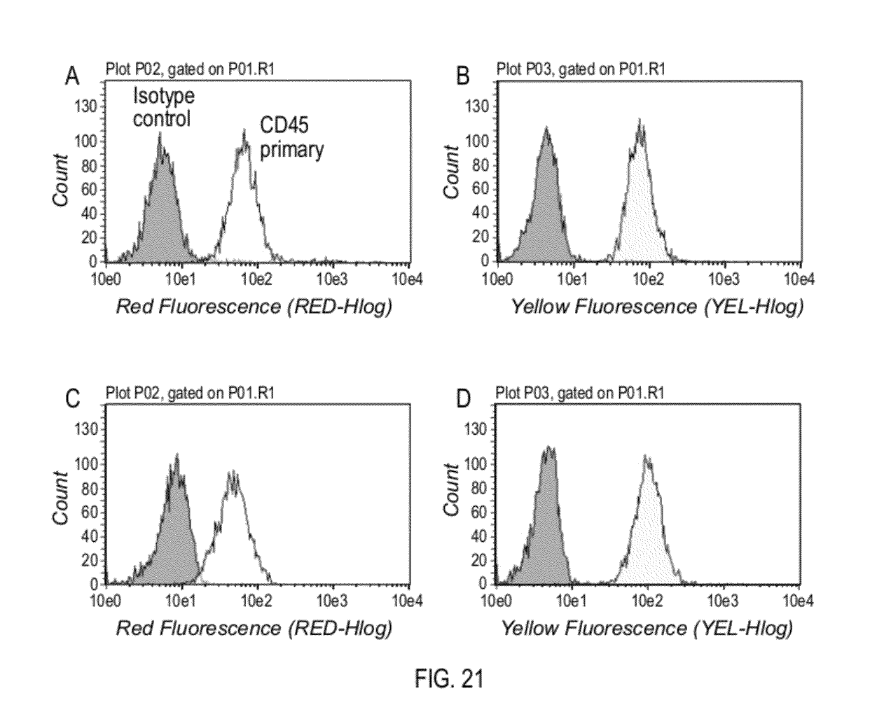

FIG. 21 illustrates the performance of antibody-FRET pair as a tandem probe. Comparison of performance of FRET probe to conventional tandem probes: Our strategy to synthesize antibody with high Stoke's shift using FRET is valuable in flow cytometry. Limited fluors are available in the Red window of flow cytometry systems (from blue lasers) due to the difficulty of making tandem conjugates like PECyS and the limited yield of these conjugates. In the example, Jurkat cells were treated with isotype control and primary CD45 antibody. These were then incubated with the same goat anti-mouse secondary antibody conjugated to multiple fluorophores, washed and subject to flow cytometry. Panels A-D represent data for a blue laser (488 nm) flow cytometer. Plot A represent probing with the N1-N5 FRET construct, B utilizes antibody from N3 only, C is data from a PE-Cy5 tandem antibody and D from GAM Cy3 conjugate. The N1-N5 FRET Probe gave better separation of populations and was easier and quicker to conjugate than PECy5 tandem used in this experiment. The N1-N5 constructs can be used with both blue and green laser flow cytometers.

### Example 24

### Protocol for Cell Viability Detection

Volumes and buffers suggested in this protocol are only representative. Protocol can be adapted to a wide variety of volumes and reaction conditions.

Prepare working solution of a dye (originally in DMSO, methanol etc.) and bring it to 1.25-150 nM in buffers such as phosphate-buffered saline (PBS) before preparation of the working solution. Pipet 100 uL of prepared cells in biological buffer or media in a reaction vessel such as a tube or microwell plate. Add 100 uL of working solution to 100 uL cell sample; mix well and incubate at the room temperature for 15 minutes. Remove supernatant without disturbing the pellet. Wash the cells with PBS with bovine serum albumin (BSA) (range 0.2-10%) per well; centrifuge plate at 1000 rpm for 5 minutes; remove the supernatant by aspiration without disturbing the cell pellet.

Procedures that Require Fixation May Use the Following Protocol

Resuspend the cell pellet in 100 uL 2% paraformaldehyde (PFA) in PBS per well. Gently mix and Incubate on ice for 10 minutes. Add 100 uL of PBS per well and wash cells. Resuspend the cell pellet in 100 uL permeabilization buffer per well. Incubate on ice for 5 minutes. Wash cells and resuspend in 200 uL PBS with BSA for analysis on flow cytometers. Samples can also be analyzed by microscopy, high content imaging, fluorometry or spectrophotometry.

### Example 25

### Cells Viability Detection

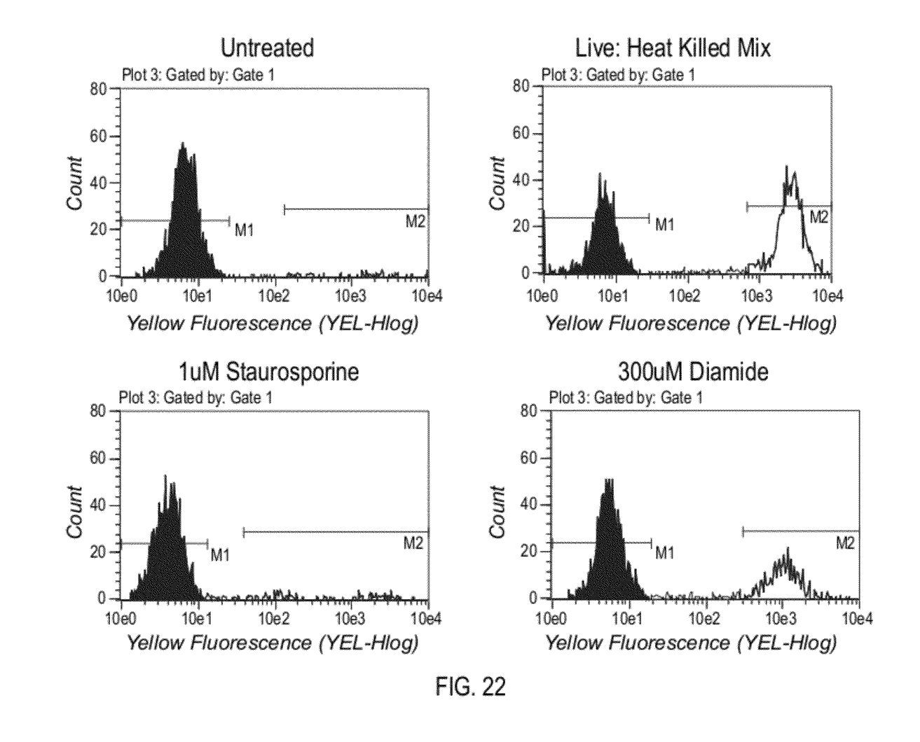

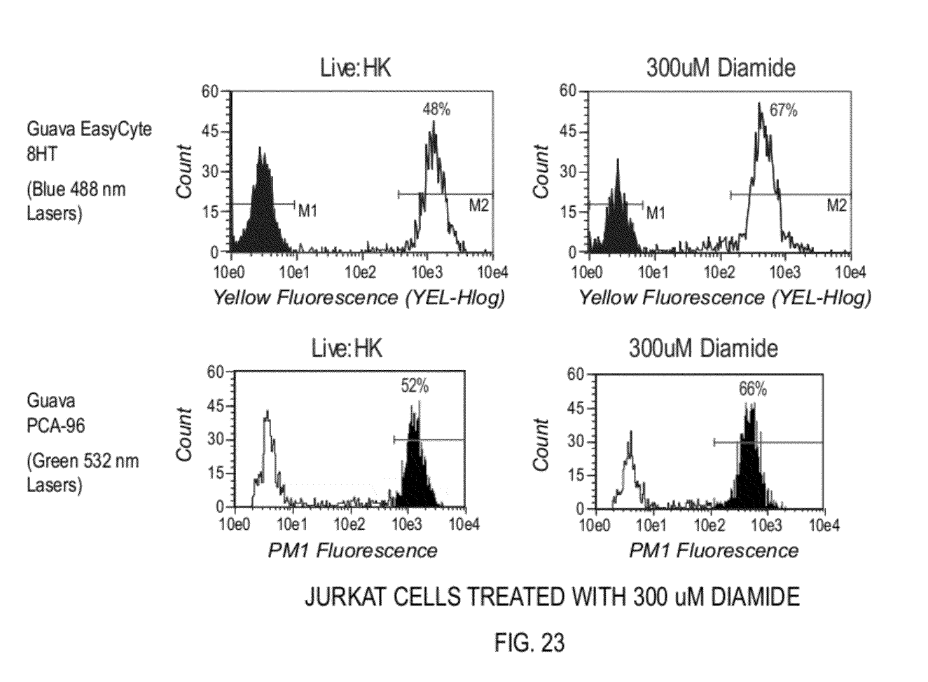

Compound N-1 was used as viability dye to detect live and dead cell populations in samples using flow cytometry. Cells were killed by heat or treated with diamide or staurosporine inducers. FIG. 22 shows that live and dead cell populations can be immediately distinguished by using Compound N-1 as a viability dye. FIG. 23 shows that the same dye N-1 can be used as a viability dye in both blue laser based instruments (488 nm) and green laser (˜532 nm) or possibly yellow laser based (˜555-560 nm) instruments.

### Example 26

### Cells Viability Detection

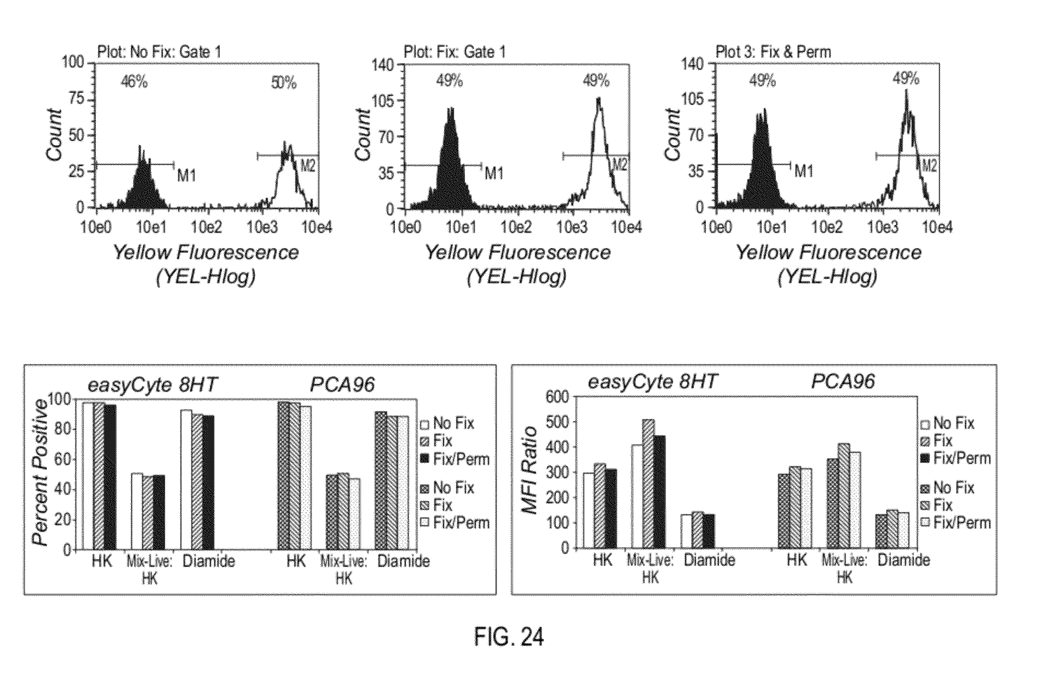

Compound N-1 was used as a viability dye. Fixation and permeabilization of sample were performed. The mixture containing live, heat killed, and diamide treated Jurkat cells were stained with Compound N-1 followed by fixation for 10 minutes and permeabilization for 5 minutes on ice as described above. Data was acquired on the EasyCyte 8HT or PCA-96 (Millipore Corporation, MA). FIG. 24 shows that equivalent percentage of populations was obtained and there was no loss of percentages of cell detected and no significant difference in fluorescence of populations after the fixation and permeabilization treatments. Hence the compound can be used in procedures where intracellular staining is required with fix and perm procedures.

### Example 27

### Cells Viability Detection

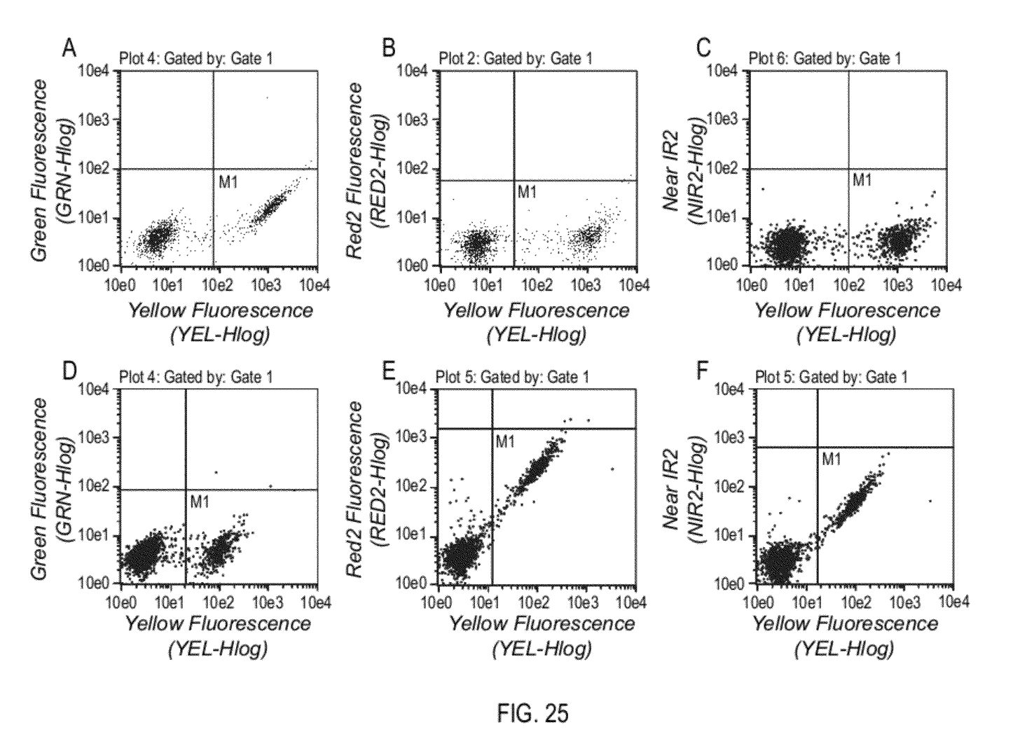

FIG. 25 shows that Compound I is one of the few options for using as an amine reactive viability dye in the yellow channel from a blue and green laser instruments. In the example shown, the dye can provide excellent separation of live and dead cells in a mix as shown in A, B and C in the yellow channel. Plots A, B and C show the fluorescence bleedthrough of the dye in adjacent channels. The data shows that minimal fluorescence is seen in the Green, Red2 and NIR2 channel when the dye is used as a viability dye. Plots D, E and F demonstrate that the closest comparable dye Invitrogen Red fluorescent reactive dye which shows good separation in the yellow channel for live and dead cells. However it shows extensive bleedthrough in the Red2, NIR2, limiting the multiplexing in experiments where amine reactive dyes and other fluorescent probes need to be used. Hence the use of Compound I as a viability dye provides better options for multiplexing experiments.

### Example 28

### Cells Viability Detection

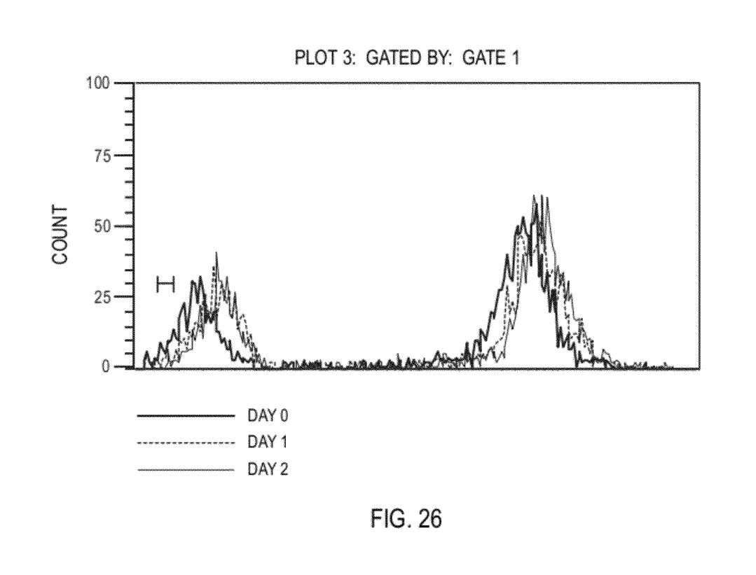

Compound N-1 was used in viability determination in which fixation was performed. Jurkat cells were untreated, heat-killed, or treated with 300 μM diamide respectively, and then stained with Compound N-1 viability dye followed by fixation for 10 minutes on ice. The samples were stored at a temperature of 2-8° C. and analyzed at 0, 24, and 48 hour post fixation respectively. FIG. 26 shows that the percentage of cells and the fluorescence detected were unchanged at 48 hour after fixation.

### Example 29

### Cells Viability Detection

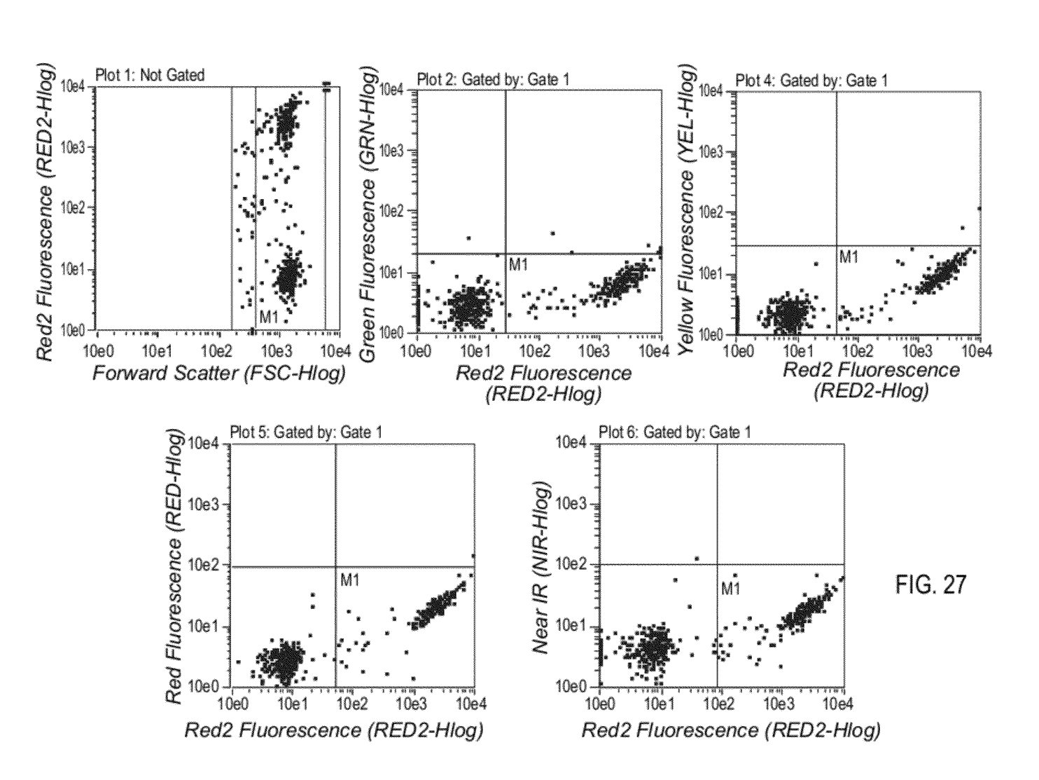

FIG. 27 shows an example using Compound N-5 as a viability dye in combination with a flow cytometer equipped with a red laser (638 nm). The sample contained cells untreated, killed by heat, or treated with inducer diamide. FIG. 27 shows that Compound N-5 as a viability dye has less bleed through in adjacent channels (in all channels <620 nm), hence it can be used in multi-parameter analysis with other fluorophores.

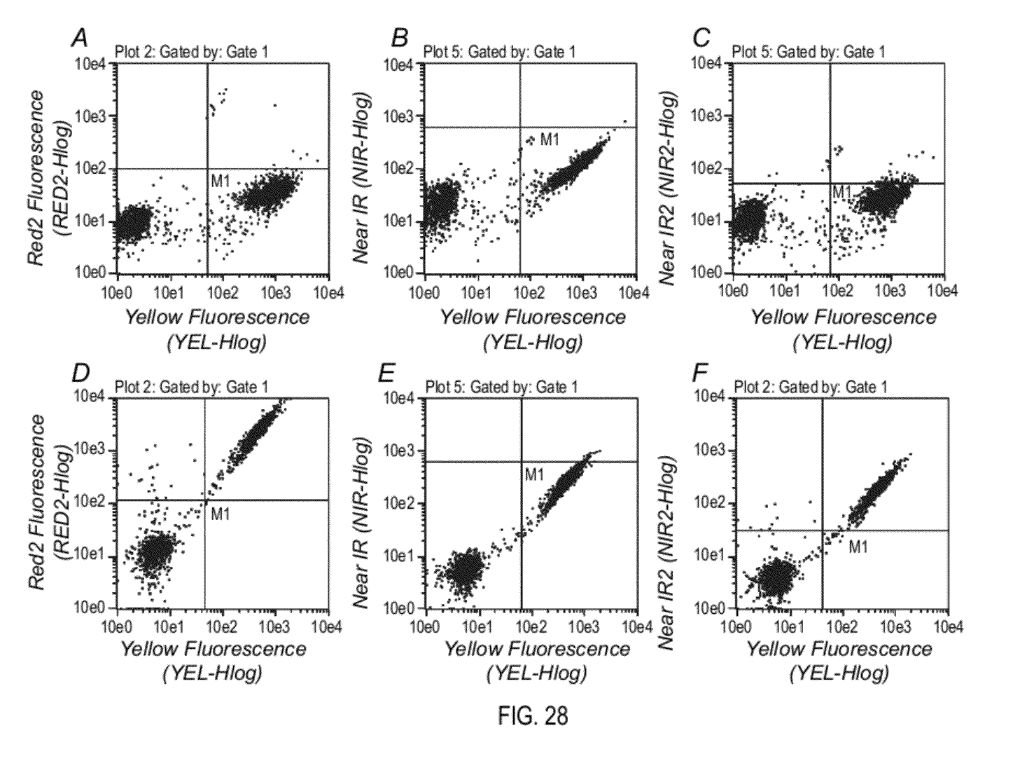

FIG. 28 shows that Compound 12 (H3) is a useful dye for cellular viability experiments. The dye has an excitation max of 510 nm and an emission max of 541 nm. The dye is excitable by both blue and green lasers and can be used on both instruments. In the example shown, the dye can provide excellent separation of live and dead cells in a mix as shown in A, B and C in the yellow channel. Plots A, B and C show the fluorescence of the dye in adjacent channels and show that minimal fluorescence is seen in the Red2 and NIR2 channel and also leave the NIR channel usable with the dye. Plots D, E and F demonstrate that the closest comparable dye Invitrogen Red fluorescent reactive dye which shows good separation in the yellow channel for live and dead cells. However it shows extensive bleedthrough in the Red2, NIR2 and NIR channel limiting the multiplexing in experiments where amine reactive dyes and other fluorescent probes need to be used. The dyes of this invention are thus better for multiplexing especially with fluorescent probes that emit in the red region of the spectrum.

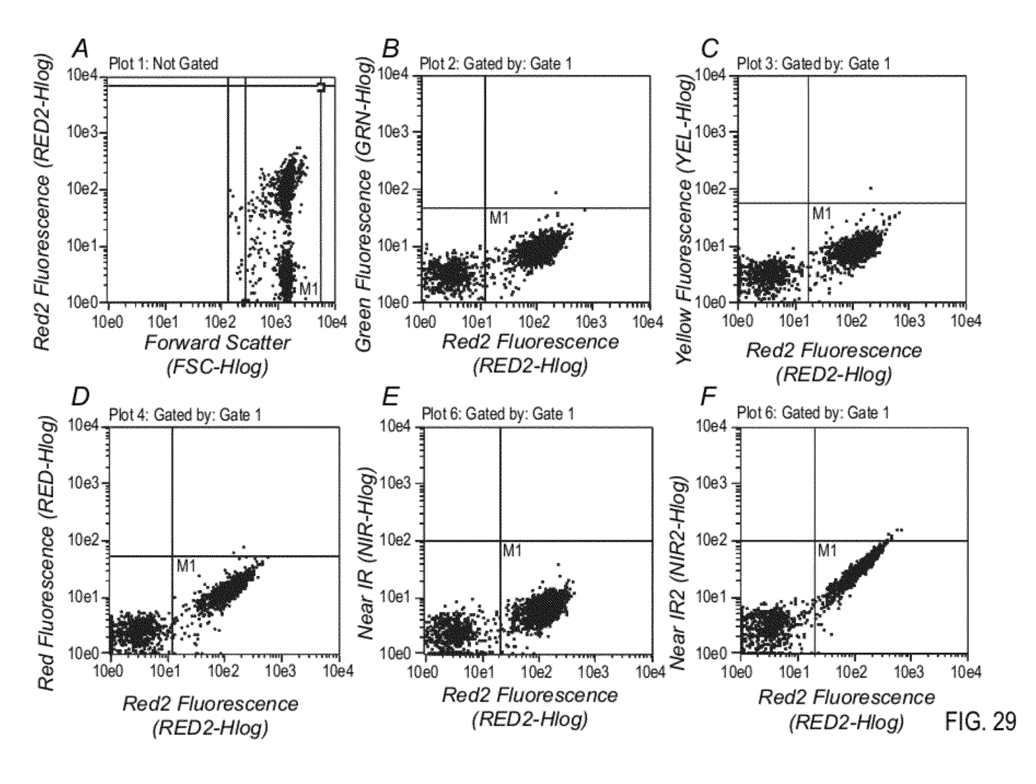

FIG. 29 shows that Compound 14 (H5) is an excellent dye for viability assays. The dye has an absorption max of 609 nm and emission max of 642 nm in methanol. A mix of live and dead cells was stained with Compound 14 using the method as described above. The dye fluoresces in the Red2 channel from a red laser (˜676 nm) and can clearly distinguish live (low fluorescence) and dead cells (high fluorescence). In addition, it shows low bleedthrough in green, yellow, red, Near IR from the blue laser and in the NIR2 channel from the red laser. This makes it an ideal dye to mix with other fluorescently labeled antibodies or fluorescent markers to create highly multiplexed assays. Other commercial dyes that fluoresce in this window show bleed through in adjacent channels when used as a viability marker.

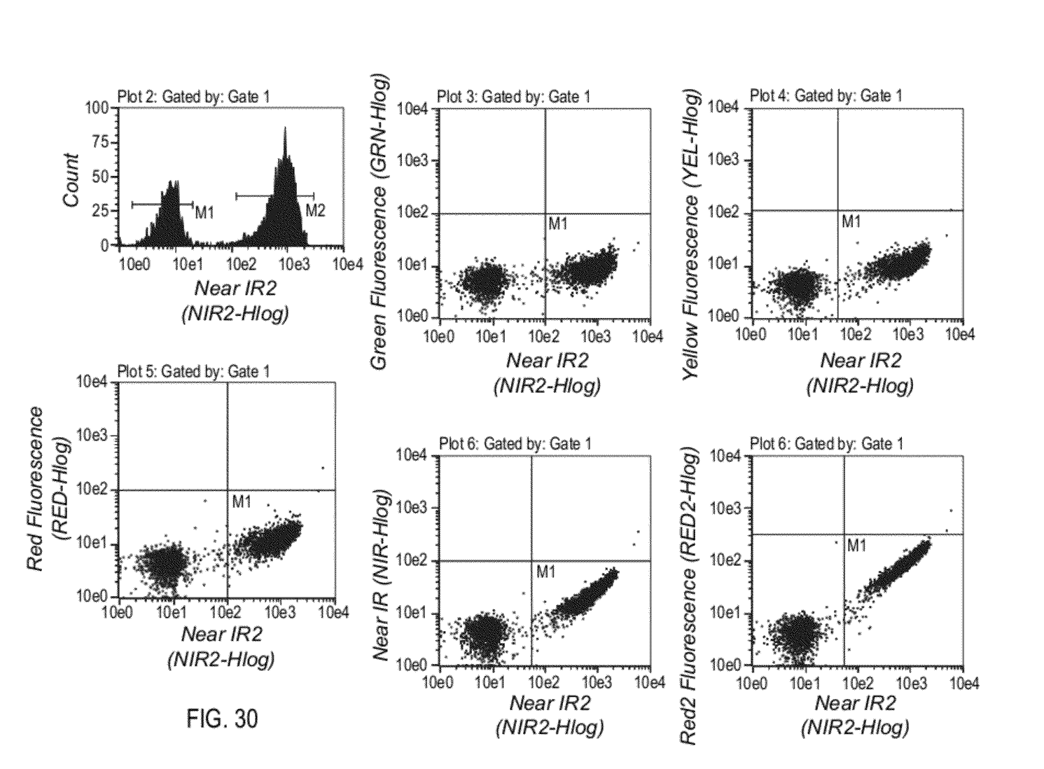

FIG. 30 shows that N7 is a good dye for use as a viability dye in the Near-IR channel from a red laser. In the example above, a mix of live and dead cells were stained with N7. The dye can clearly distinguish live (low fluorescence) and dead cells (high fluorescence). In addition, it shows low bleedthrough in green (˜525 nm), yellow (˜576 nm), red (˜676 nm), Near IR (>750 nm) from the blue laser (˜488 nm) and Red2 (˜676 nm) channel from the red laser. This makes it an ideal dye to mix with other fluorescent markers to create multiplexed assays especially since there are fewer conjugates available to use in the NIR2 channels. This dye allows the use of the NIR2 channel for viability leaving all the other channels free in a multiplexed assay.

### Example 30

### Use as Dyes for Cell Painting Applications

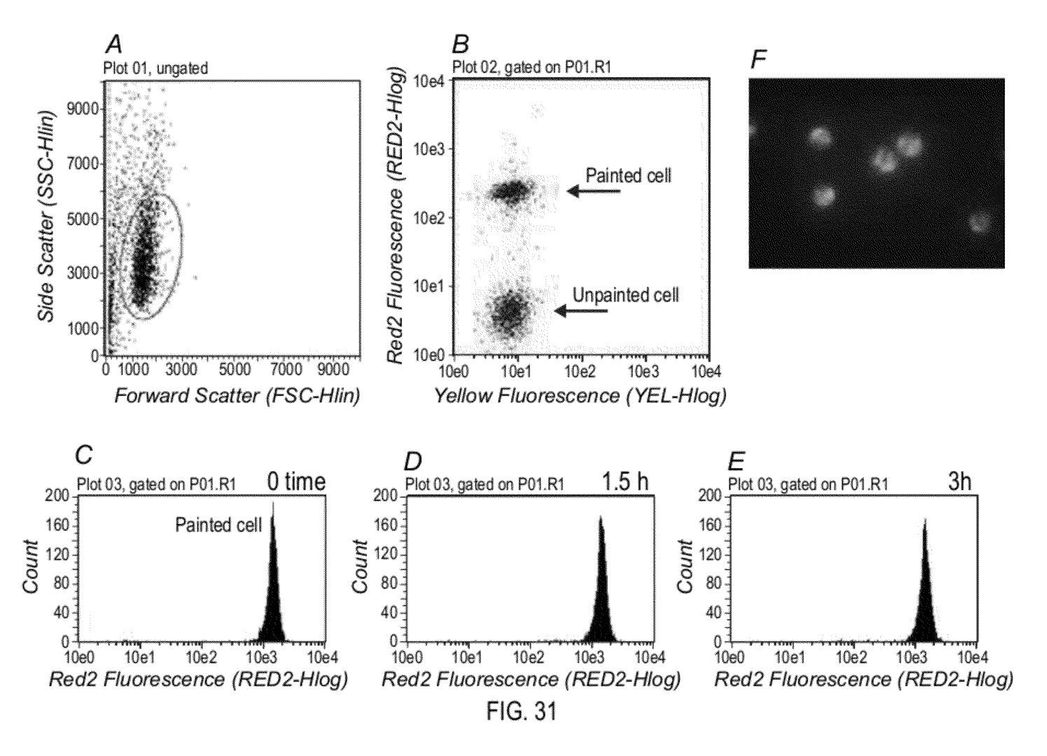

Compound 19 demonstrated good utility for labeling and painting cells. The fluorescent characteristics of this dye (Ex 650 nm, Em 674 nm in methanol) and its emission in the Red2 region off a red laser and its low bleedthrough in adjacent channels make it a useful dye for cell painting applications where additional probes can be in channels from a blue laser and painted cells can be analyzed for multiple impacts. FIG. 31 shows that painted cells had good retention of dye for the 3 h time period studied (C to E). Microscopy reveals that the dye was localized intracellularly (F). Similarly compound 18 (Ex 551 nm, Em 574 nm in methanol) also showed good utility for painting cells and these cells fluoresced in the yellow channel from the blue laser. Compound 18 demonstrates utility for cell painting applications where pairing with probes from a red laser is required or a green fluorescent probe from the blue 488 nm laser is required.

Those skilled in the art will appreciate that various other modifications may be made within the spirit and scope of the invention. All these or other variations and modifications are contemplated by the inventors and within the scope of the invention.

What is claimed is:

1. A compound of the general formula I: 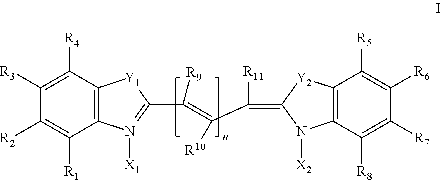
where:
R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;
R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;
Y₁ and Y₂ are independently selected from the group consisting of O, N, S, and —CR′R″— where R′ and R″ are independently H or C₁-C₁₈ alkyl, and at least one of Y₁ and Y₂ is O, S, or N;
X₁ and X₂ are independently selected from the group consisting of optionally substituted alkyl, optionally substituted heteroalkyl, and optionally substituted alkylaryl, wherein at least one of X₁ and X₂ is substituted alkylaryl comprising on the aryl component a substituted alkyl or heteroalkyl comprising a carboxylic acid substituent or derivative thereof; and
n is 1, 2, or 3,
or an isomer, ester, amide, acid halide, acid anhydride, and/or salt thereof.

2. The compound of claim 1 wherein one of Y₁ and Y₂ is O, and one or both of X₁ and X₂ is or are substituted alkylaryl which comprises on the aryl component a substituted alkyl or heteroalkyl substituent comprising a carboxylic acid substituent, or an isomer, ester, amide, acid halide, and/or salt thereof, or a mixture of any thereof.

3. The compound of claim 1 wherein both of Y₁ and Y₂ are O, and one or both of X₁ and X₂ is or are substituted alkylaryl comprising on the aryl component a substituted alkyl or heteroalkyl substituent comprising a carboxylic acid substituent, or an isomer, ester, amide, acid halide, and/or salt thereof, or a mixture of any thereof.

4. The compound of claim 1 wherein one or both of Y₁ and Y₂ is or are O, and at least one of R₁ to R₈ is SO₃H, or an isomer, ester, amide, acid halide, and/or salt thereof, or a mixture of any thereof.

5. The compound of claim 1 wherein one or both of Y₁ and Y₂ is or are O, and at least one of R₃ and R₆ is —SO₃H, or an isomer, ester, amide, acid halide, and/or salt thereof, or a mixture of any thereof.

6. The compound of claim 1 wherein one or both of Y₁ and Y₂ is or are O, and R₃ and R₄, and R₅ and R₆ taken together respectively form a 6-membered ring optionally substituted by SO₃H or a derivative thereof, and R₁-R₂ and R₇-R₁₁ are independently H.

7. The compound of claim 1 wherein one of Y₁ and Y₂ is O, and one of Y₁ and Y₂ is C(CH₃)₂.

8. The compound of claim 1 wherein both of Y₁ and Y₂ are O, and both of X₁ and X₂ are the same and represent a group of the formula II: 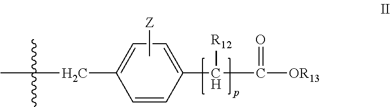
where Z is selected from the group consisting of H, SO₃H, optionally substituted alkyl, and optionally substituted phenyl; p is a number from 1 to 18; R₁₂ is H or —CH₃, and R₁₃ is selected from the group consisting of the formulas III-a, III-b, III-c, III-d, and III-e:

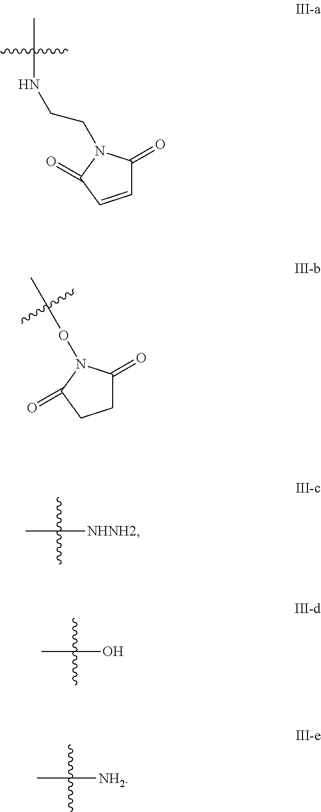

9. The compound of claim 1 wherein both of Y₁ and Y₂ are O, one of X₁ and X₂ is a group of the formula II: 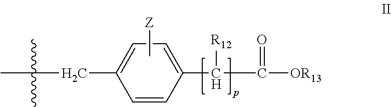
where Z is selected from the group consisting of H, SO₃H, optionally substituted alkyl, and optionally substituted phenyl; p is a number from 1 to 18; R₁₂ is H or —CH₃, and R₁₃ is selected from the group consisting of the formulas III-a, III-b, III-c, III-d, and III-e: 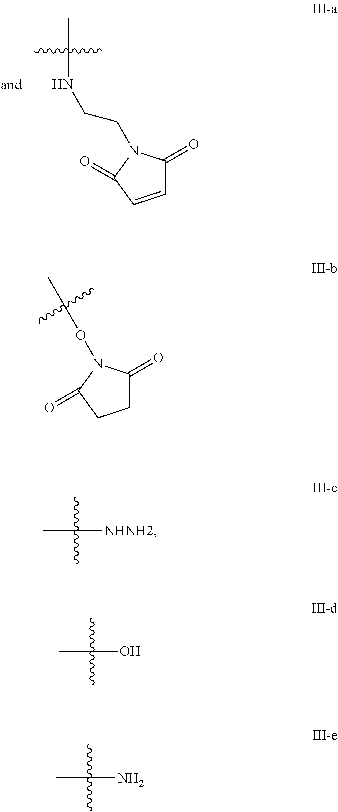
one of X₁ and X₂ is a group of the formula IV: 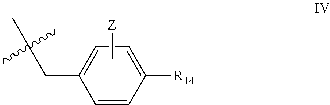
where R₁₄ is an optionally substituted alkyl or optionally substituted phenyl group, and Z is selected from the group consisting of H, SO₃H, optionally substituted alkyl, and optionally substituted phenyl.

10. The compound of claim 1 wherein one of Y₁ and Y₂ is O, one of Y₁ and Y₂ is —C(CH₃)₂—, and X₁ and X₂ are the same and represent a group of the formula II: 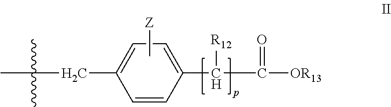
where Z is selected from the group consisting of H, SO₃H, optionally substituted alkyl, and optionally substituted phenyl; p is a number from 1 to 18; R₁₂ is H or —CH₃, and R₁₃ is selected from the group consisting of the formulas III-a, III-b, III-c, III-d, and III-e:

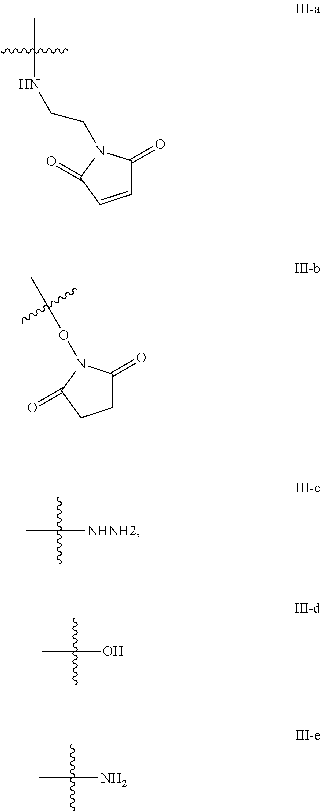

11. The compound of claim 1 wherein one of Y₁ and Y₂ is O, one of Y₁ and Y₂ is —C(CH₃)₂—, one of X₁ and X₂ is a group of the formula II: 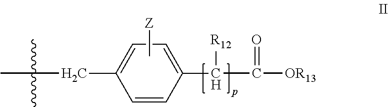
where Z is selected from the group consisting of H, SO₃H, optionally substituted alkyl, and optionally substituted phenyl; p is a number from 1 to 18; R₁₂ is H or —CH₃, and R₁₃ is selected from the group consisting of the formulas III-a, III-b, III-c, III-d, and III-e: 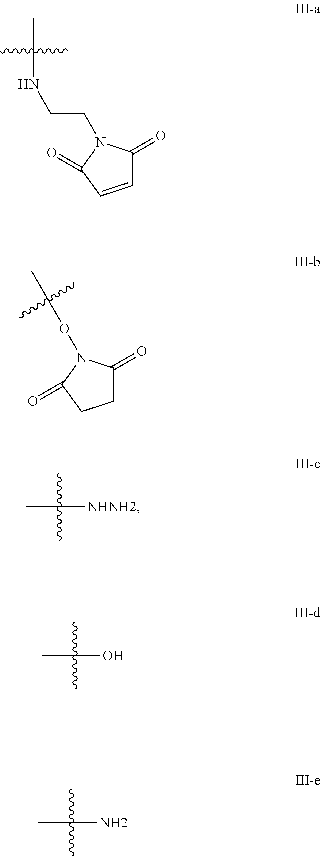
and
one of X₁ and X₂ is a group of the formula IV: 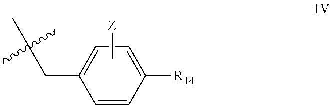
where R₁₄ is an optionally substituted alkyl or optionally substituted phenyl group, and Z is selected from the group consisting of H, SO₃H, optionally substituted alkyl, and optionally substituted phenyl.

12. The compound of claim 1, which is selected from the group consisting of: 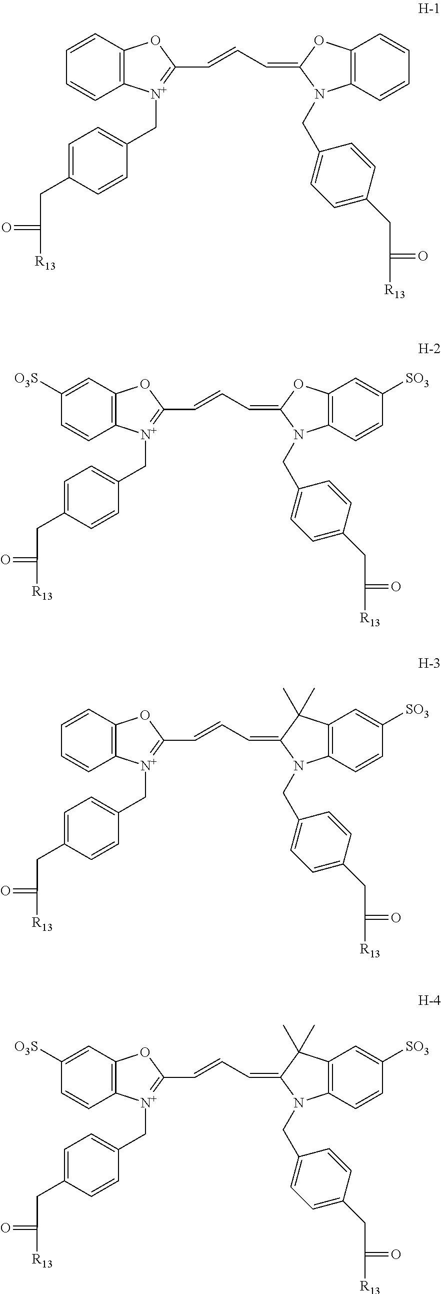 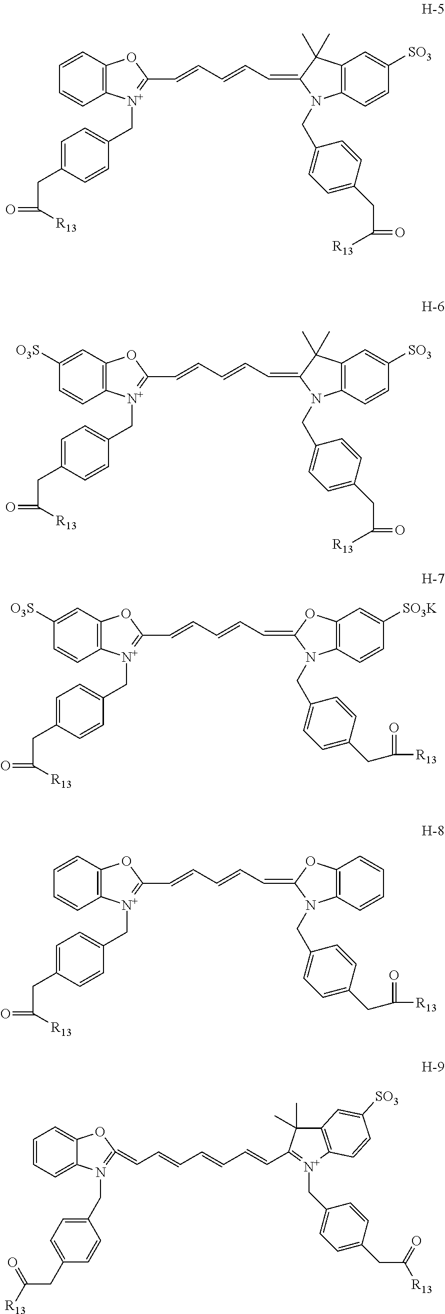 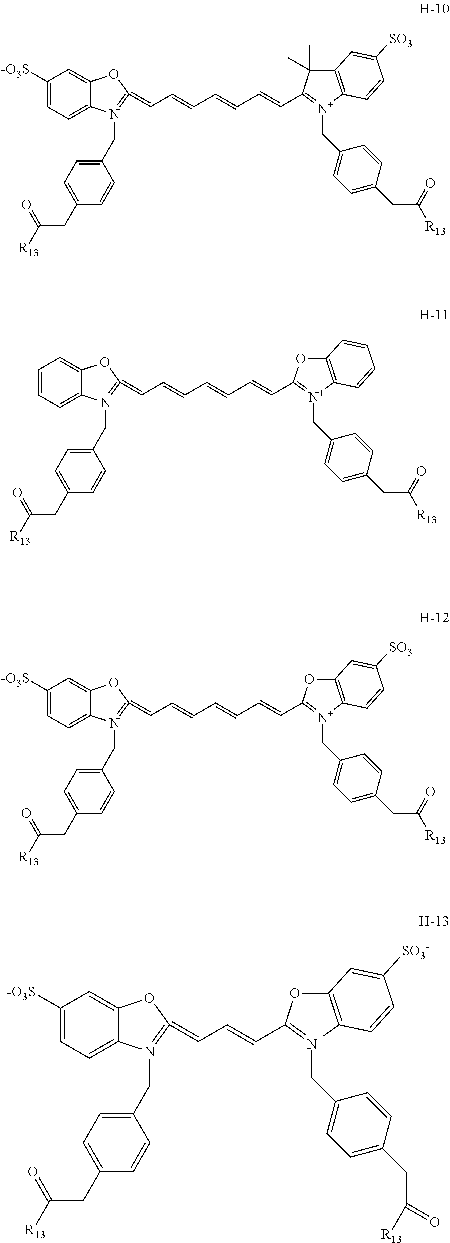 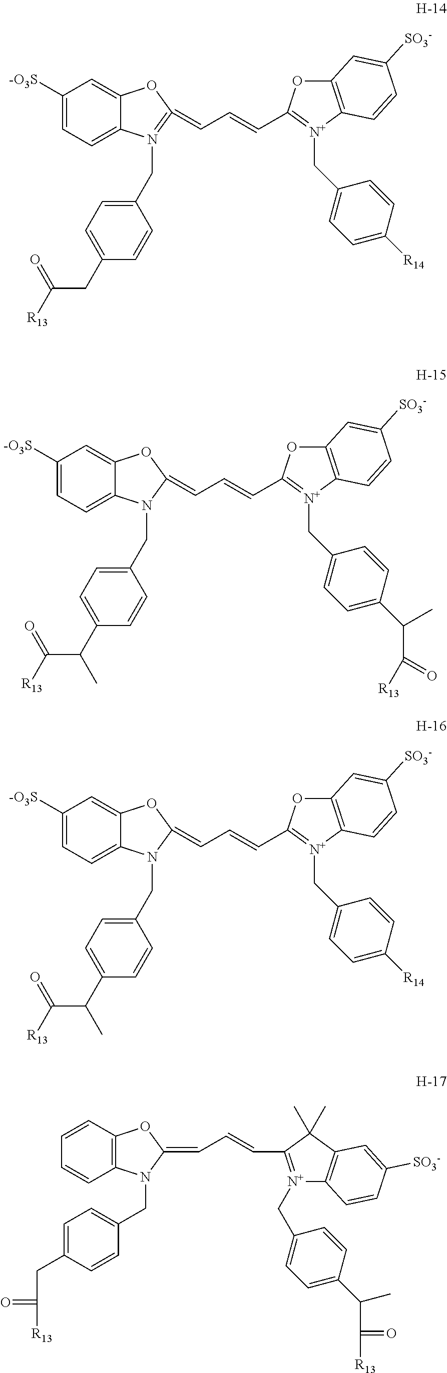
where R₁₃ is selected from the group consisting of the formulas of III-a, III-b, III-c, III-d, and III-e: 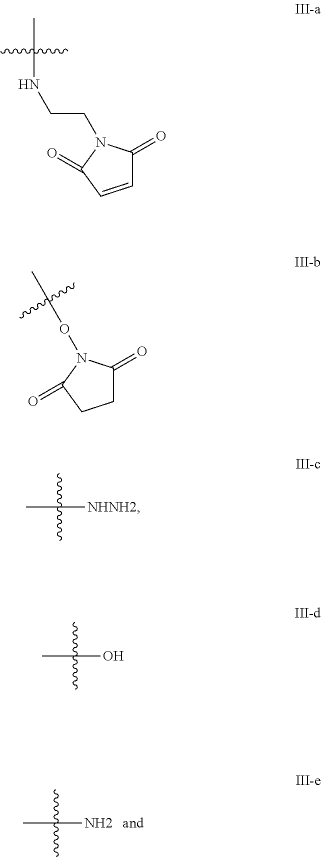
R₁₄ is an optionally substituted alkyl or optionally substituted phenyl group.

13. A compound of the general formula I: 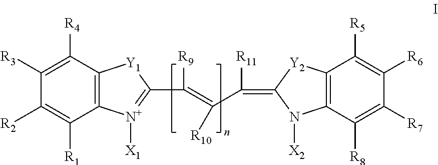
where:
R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;
R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;
Y₁ and Y₂ are independently selected from the group consisting of O, N, S, and —CR′R″— where R′ and R″ are independently H or C₁-C₁₈ alkyl;
X₁ is selected from the group consisting of NU-1 to NU-30: 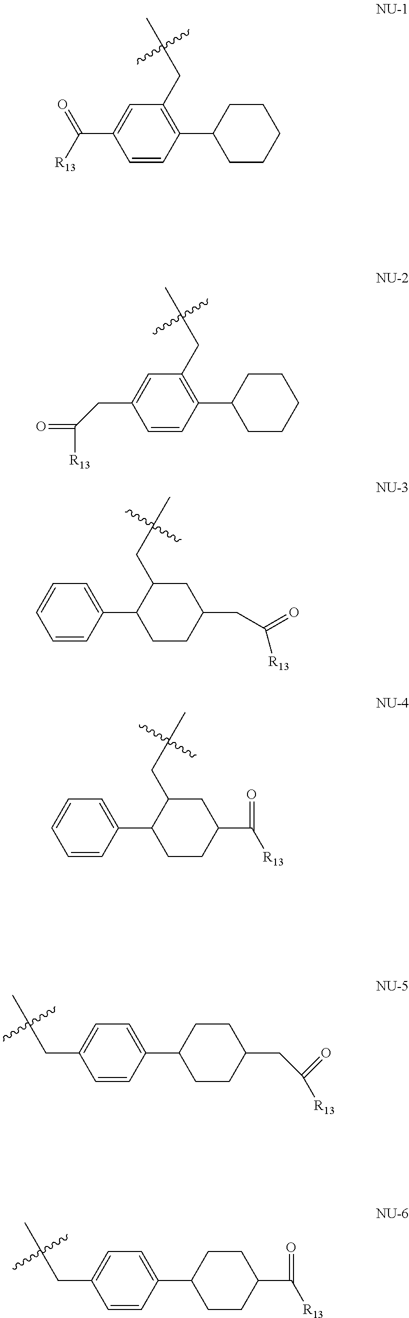 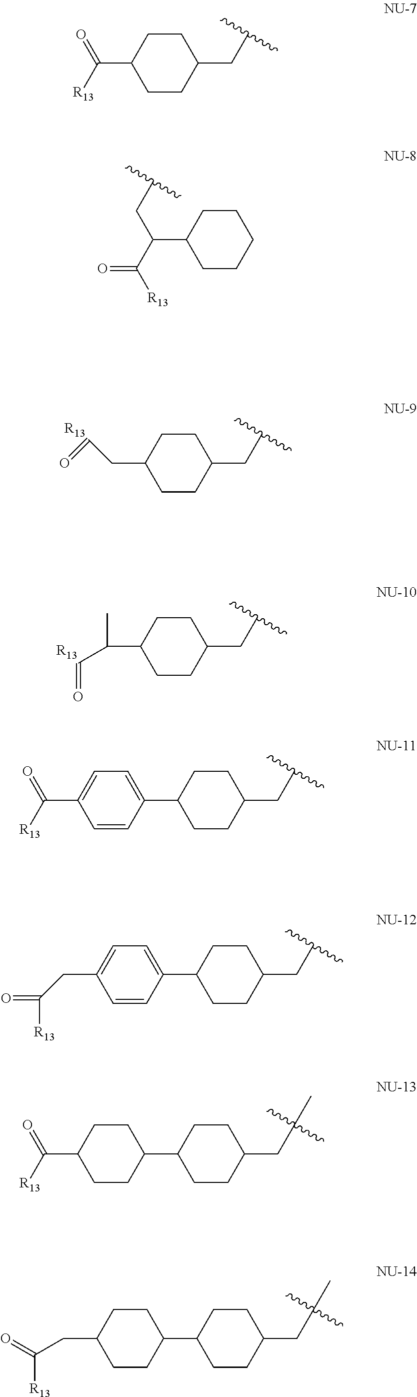 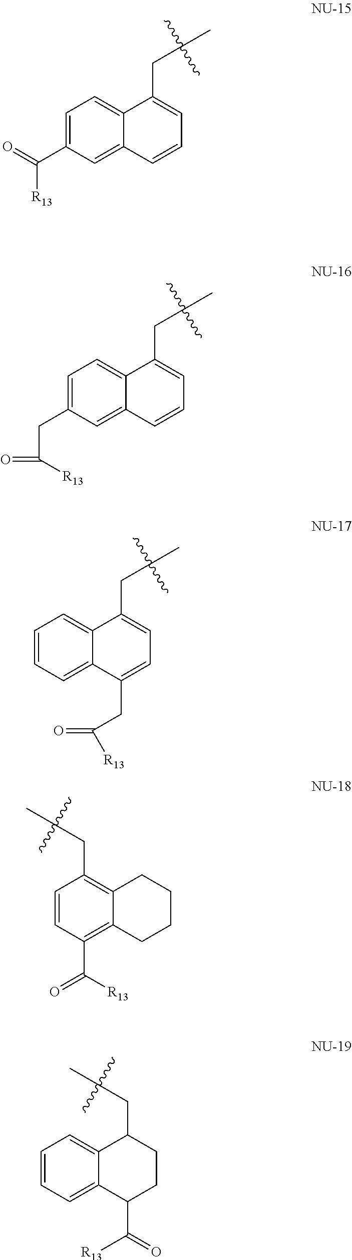 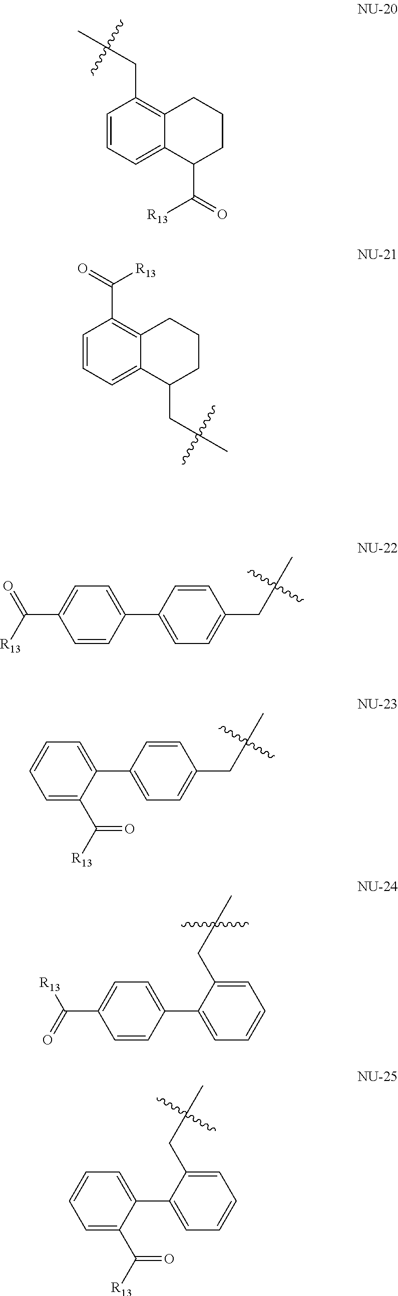 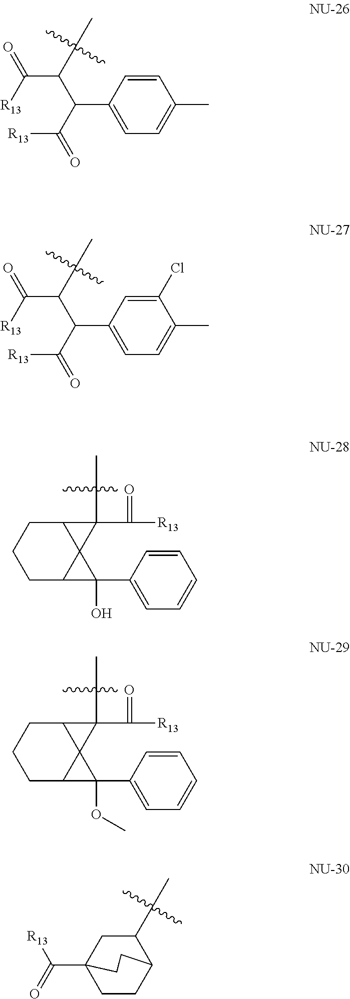
where R₁₃ is selected from the group consisting of the formulas of III-a, III-b, III-c, III-d, and III-e: 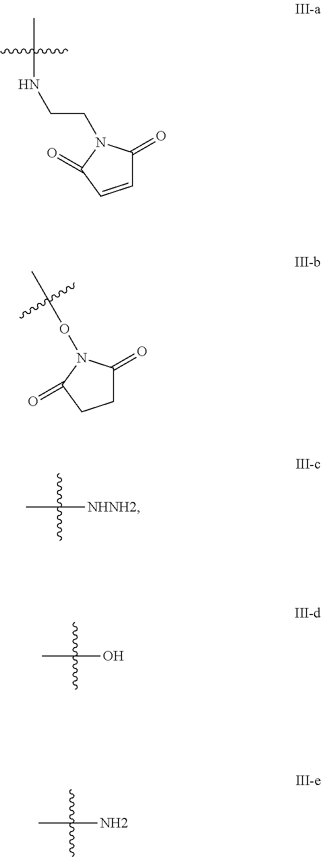
X₂ is the same as X₁, or a group of the formula IV below: 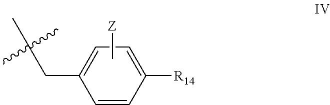
where R₁₄ is an optionally substituted alkyl or optionally substituted phenyl group, and Z is selected from the group consisting of H, SO₃H, optionally substituted alkyl, and optionally substituted phenyl; and
n is 1, 2, or 3,
or an isomer, ester, amide, acid halide, acid anhydride, and/or salt thereof.

14. The compound of claim 13 wherein X₂ is the same as X₁.

15. The compound of claim 13 wherein X₂ is a group of the formula IV. 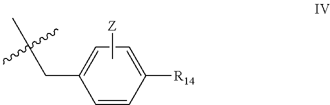
where R₁₄ is an optionally substituted alkyl or optionally substituted phenyl group, and Z is selected from the group consisting of H, SO₃H, optionally substituted alkyl, and optionally substituted phenyl.

16. The compound of claim 13 wherein Y₁ and Y₂ are independently O, N, S, or —CR′R″— where R′ and R″ are independently H or C₁-C₁₈ alkyl.

17. The compound of claim 13 wherein both of Y₁ and Y₂ are —CR′R″— where R′ and R″ are independently H or C₁-C₁₈ alkyl.

18. The compound of claim 13 wherein both of Y₁ and Y₂ are O.

19. The compound of claim 13 wherein one of Y₁ and Y₂ is O, N, or S, and one of Y₁ and Y₂ is —CR′R″— where R′ and R″ are independently H or C₁-C₁₈ alkyl.

20. The compound of claim 13 wherein at least one of R₁ to R₈ is SO₃H, or an isomer, ester, amide, acid halide, and/or salt thereof, or a mixture of any thereof.

21. The compound of claim 13 wherein at least one of R₃ and R₆ is —SO₃H, or an isomer, ester, amide, acid halide, and/or salt thereof, or a mixture of any thereof.

22. The compound of claim 13 wherein R₃ and R₄, and R₅ and R₆ taken together respectively form a 6-membered ring optionally substituted by SO₃H or a derivative thereof, and R₁-R₂ and R₇-R₁₁ are independently H.

23. The compound of claim 13 which has a structure of the formula of I-a: 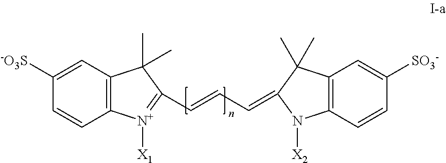
where n is 1, 2, or 3, and X₁ and X₂ are the same and selected from the group consisting of NU-1 to NU-30 defined as above.

24. The compound of claim 13 which has a structure of the formula of I-a: 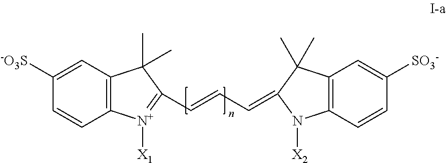
where n is 1, 2, or 3, X₁ is selected from the group consisting of NU-1 to NU-30 defined as above, and X₂ is a group having the formula IV as defined.

25. The compound of claim 13 which has a structure of the formula I-b: 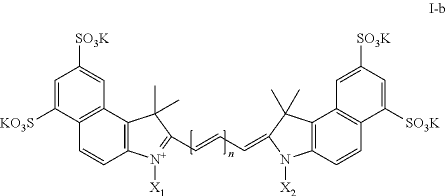
where n is 1, 2, or 3, and X₁ and X₂ are the same and selected from the group consisting of NU-1 to NU-30 defined as above.

26. The compound of claim 13 which has a structure of the formula I-b: 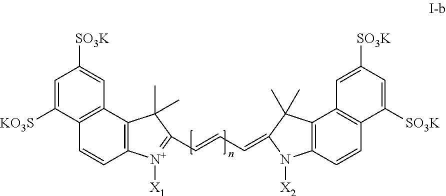
where n is 1, 2, or 3, X₁ is selected from the group consisting of NU-1 to NU-30 defined as above, and X₂ is a group having the formula IV defined above.

27. A dye pair comprising:
a first fluorescent compound coupled to a first biomolecular segment;
a second fluorescent compound coupled to a second biomolecular segment;
wherein said first fluorescent compound has a first excitation spectrum and a first emission spectrum, said second fluorescent compound has a second excitation spectrum and a second emission spectrum, and said first emission spectrum of the first compound at least partially overlaps the second excitation spectrum of the second fluorescent compound.

28. The dye pair of claim 27 wherein the first and second biomolecular segments are on a same biomolecule.

29. The dye pair of claim 28 wherein said biomolecule comprises a protein.

30. The dye pair of claim 28 wherein said biomolecule comprises an antibody.

31. The dye pair of claim 27 wherein the first biomolecular segment is on a first biomolecule and the second biomolecular segment is on a second biomolecule different from the first biomolecule.

32. The dye pair of claim 31 wherein the first and second biomolecules comprise protein-protein, protein-oligosaccharide, oligosaccharide-oligosaccharide, protein-ligand.

33. The dye pair of claim 27 wherein at least one of the first and second fluorescent compounds has the general formula I, 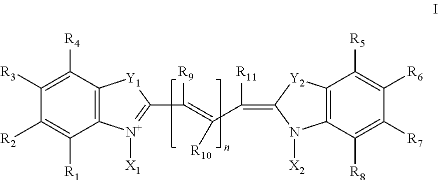
where:
R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;
R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;
Y₁ and Y₂ are independently selected from the group consisting of O, N, S, and —CR′R″— where R′ and R″ are independently H or C₁-C₁₈ alkyl,
X₁ and X₂ are independently selected from the group consisting of optionally substituted alkyl, optionally substituted heteroalkyl, and optionally substituted alkylaryl, wherein at least one of X₁ and X₂ is substituted alkylaryl comprising on the aryl component a substituted alkyl or heteroalkyl comprising a carboxylic acid substituent or derivative thereof; and
n is 1, 2, or 3.

34. The dye pair of claim 27 wherein the first fluorescent compound has the formula of N-1 or N-2, and the second fluorescent compound has the formula of N-5 or N-6: 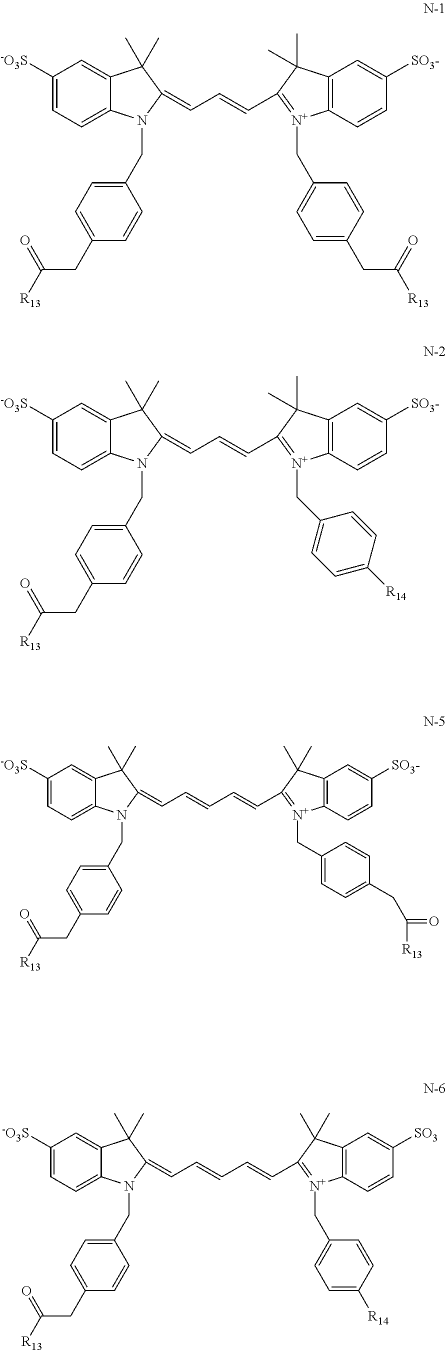
where R₁₃ is selected from the group consisting of the formulas III-a, III-b, III-c, III-d, and III-e, and R₁₄ is an optionally substituted alkyl or optionally substituted phenyl group,

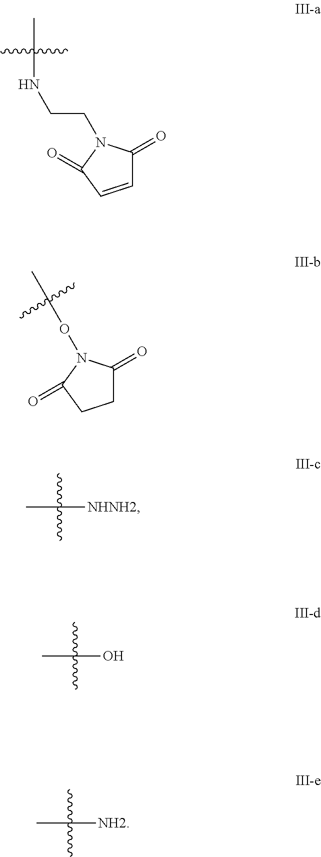

35. The dye pair of claim 27 wherein the first fluorescent compound has the formula of N-5 or N-6, and the second fluorescent compound has the formula of N-9 or N-10: 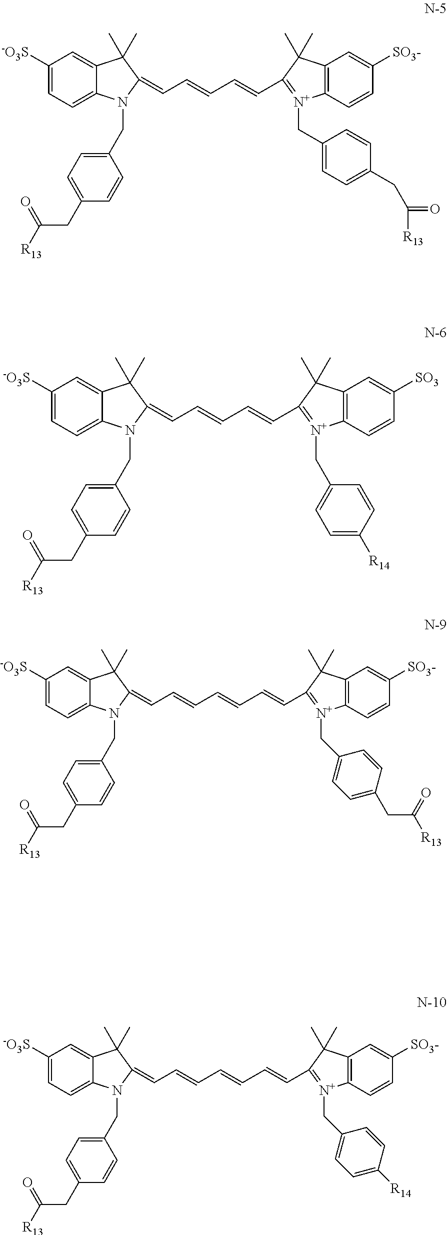
where R₁₃ is selected from the group consisting of the formulas III-a, III-b, III-c, III-d, and III-e, and R₁₄ is an optionally substituted alkyl or optionally substituted phenyl group,

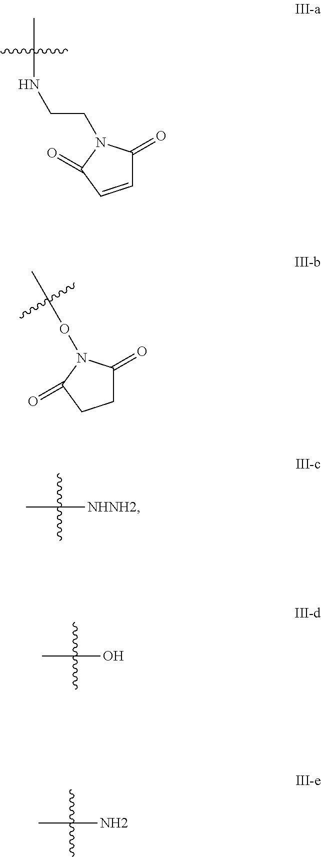

36. A method of preparing tandem probe comprising the step of coupling a first fluorescent compound and a second fluorescent compound to a probe simultaneously, wherein said first fluorescent compound has a first excitation spectrum and a first emission spectrum, said second fluorescent compound has a second excitation spectrum and a second emission spectrum, and said first emission spectrum of the first compound at least partially overlaps the second excitation spectrum of the second fluorescent compound.

37. The method of claim 36 wherein the probe comprises a biomolecule.

38. The method of claim 36 wherein the probe comprises a non-fluorescent protein or biomolecule.

39. The method of claim 36 wherein the probe comprises a non-fluorescent antibody.

40. A method of determining a proportion of cells with intact membranes in a sample containing cells with damaged membranes and cells with intact membranes, comprising the steps of:
incubating a fluorescent cyanine compound having the general formula I with a sample containing cells with intact membranes and cells with damaged membranes, thereby the cyanine compound is coupled to the intact cells and the damaged cells respectively;
causing the cyanine compound coupled to the intact cells and the cyanine compound coupled to the damaged cells to emit fluorescence;
detecting the fluorescence emitted by the cyanine compound coupled to the intact cells and the damaged cells;
determining a difference in intensity of the fluorescence detected; and
determining the proportion of the cells with intact membranes and damaged membranes in the sample based on the difference in the intensity of the fluorescence; 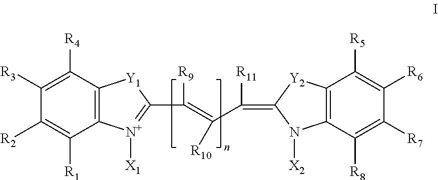
where:
R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;
R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;
Y₁ and Y₂ are independently selected from the group consisting of O, N, S, and —CR′R″— where R′ and R″ are independently H or C₁-C₁₈ alkyl;
X₁ and X₂ are independently selected from the group consisting of optionally substituted alkyl, optionally substituted heteroalkyl, and optionally substituted alkylaryl, wherein at least one of X₁ and X₂ is substituted alkylaryl comprising on the aryl component a substituted alkyl or heteroalkyl comprising a carboxylic acid substituent or derivative thereof; and
n is 1, 2, or 3,
or an isomer, ester, amide, acid halide, acid anhydride, and/or salt thereof.

41. The method of claim 40 wherein the step of detecting the fluorescence is carried out by flow cytometry, microscopy, imaging, or fluorescence plate readers.

42. The method of claim 40 further comprising the step of permeabilizing the cells in the sample.

43. The method of claim 40 further comprising the step of fixing the cells in the sample.

44. The method of claim 40 further comprising the step of coupling the cells in the sample with a single or multiple additional probes labeled with a fluorescent moiety to detect cellular physiology, extra- or intra-cellular protein or biomolecule.

45. The method of claim 40 wherein the detection with the cyanine dye involves wash steps or no wash-steps.

46. A conjugate comprising a compound of the general formula I: 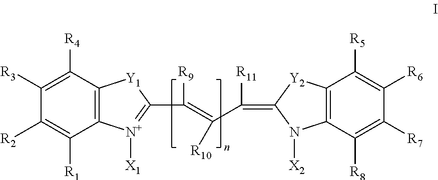
where:
R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;
R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;
Y₁ and Y₂ are independently selected from the group consisting of O, N, S, and —CR′R″— where R′ and R″ are independently H or C₁-C₁₈ alkyl, and at least one of Y₁ and Y₂ is O, S, or N;
X₁ and X₂ are independently selected from the group consisting of optionally substituted alkyl, optionally substituted heteroalkyl, and optionally substituted alkylaryl, wherein at least one of X₁ and X₂ is substituted alkylaryl comprising on the aryl component a substituted alkyl or heteroalkyl comprising a carboxylic acid substituent or derivative thereof; and
n is 1, 2, or 3,
or an isomer, ester, amide, acid halide, acid anhydride, and/or salt thereof;
wherein the compound is coupled to a species selected from a biomolecule, a synthetic dye, a substrate, a probe, a linker, a target, a low affinity false target, a member of a binding pair, a small molecule, a polymer, an inert surface, a microparticle, a nanoparticle, and/or an optically active species.

47. The conjugate of claim 46 wherein the biomolecule comprises proteins, peptides, polynucleotides, polysaccharides, antibodies, antibody fragments, nucleic acid, triglycerides, lipoproteins, and lectins.

48. A conjugate comprising a compound of the general formula I 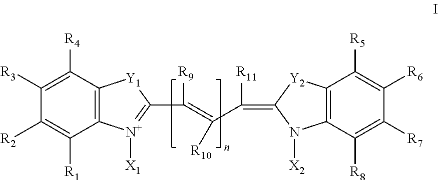
where:
R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;
R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;
Y₁ and Y₂ are independently selected from the group consisting of O, N, S, and —CR′R″— where R′ and R″ are independently H or C₁-C₁₈ alkyl;
X₁ is selected from the group consisting of NU-1 to NU-30: 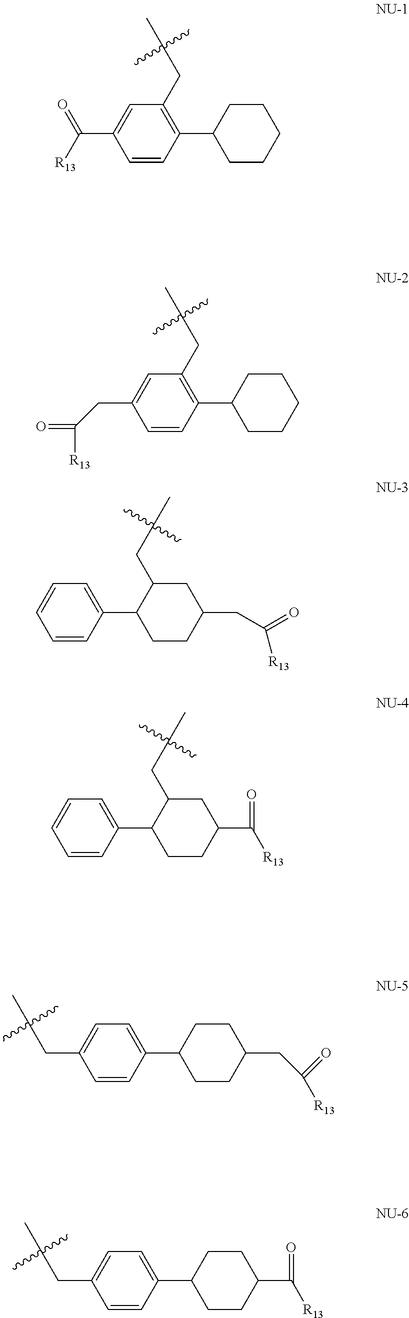 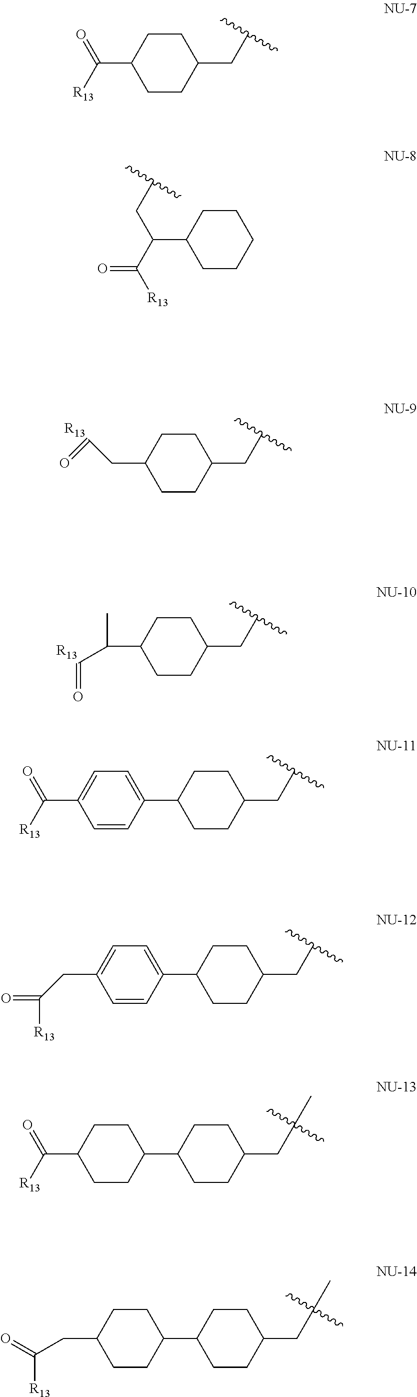 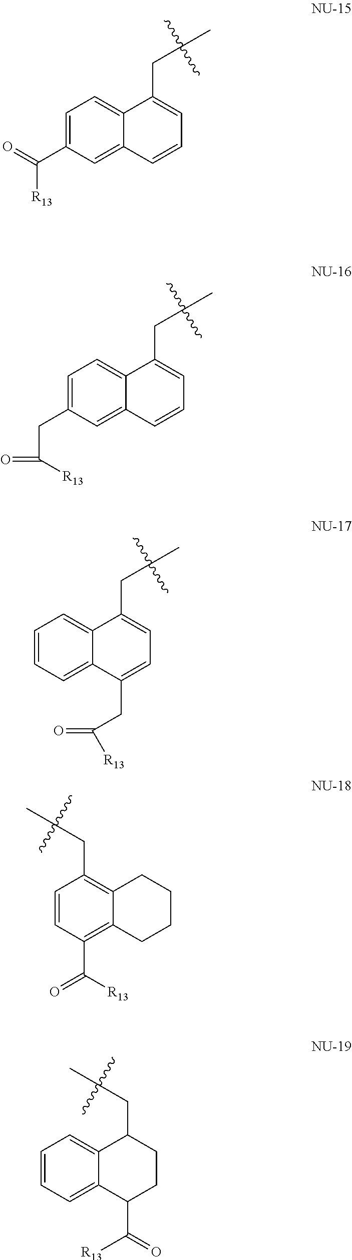 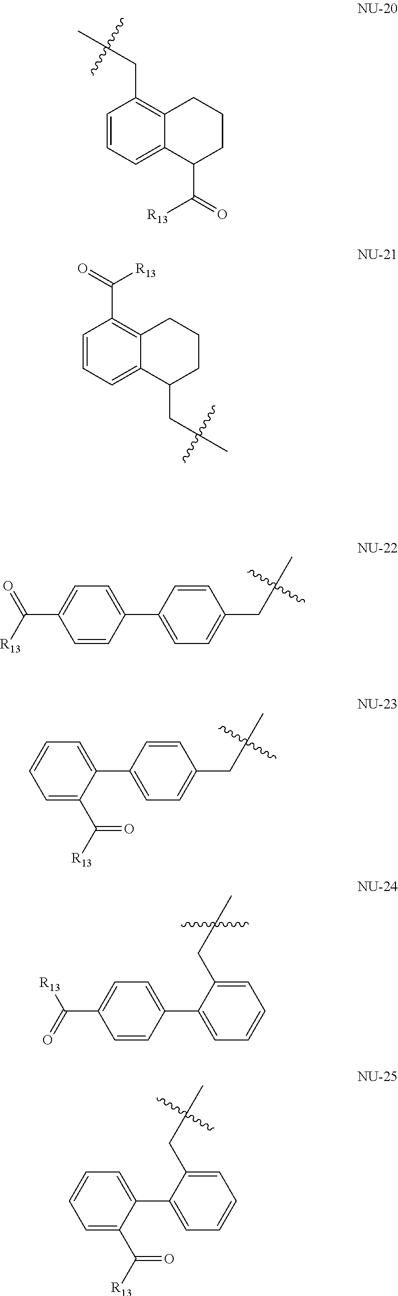 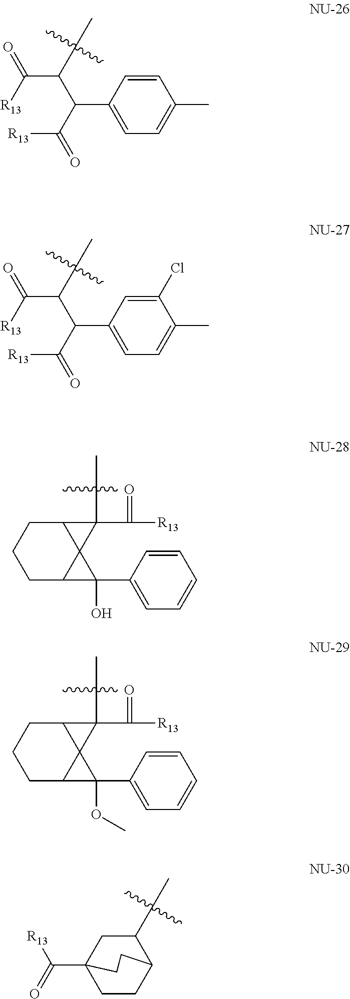
where R₁₃ is selected from the group consisting of the formulas of III-a, III-b, III-c, III-d, and III-e: 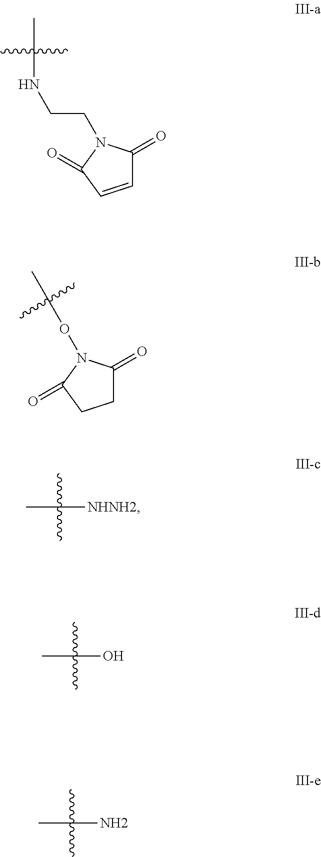
X₂ is the same as X₁, or a group of the formula IV below: 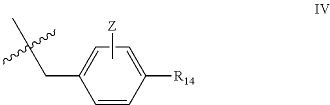
where R₁₄ is an optionally substituted alkyl or optionally substituted phenyl group, and Z is selected from the group consisting of H, SO₃H, optionally substituted alkyl, and optionally substituted phenyl; and
n is 1, 2, or 3,
or an isomer, ester, amide, acid halide, acid anhydride, and/or salt thereof;
wherein the compound is coupled to a species selected from a biomolecule, a synthetic dye, a substrate, a probe, a linker, a target, a low affinity false target, a member of a binding pair, a small molecule, a polymer, an inert surface, a microparticle, a nanoparticle, and/or an optically active species.

49. The conjugate of claim 48 wherein the biomolecule comprises proteins, peptides, polynucleotides, polysaccharides, antibodies, antibody fragments, nucleic acid, triglycerides, lipoproteins, and lectins.

50. A conjugate comprising a compound of the general formula I: 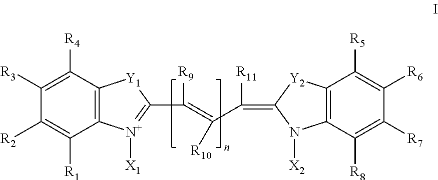
where:
R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;
R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;
Y₁ and Y₂ are independently selected from the group consisting of O, N, S, and —CR′R″— where R′ and R″ are independently H or C₁-C₁₈ alkyl;
X₁ is a group of the formula II-b: 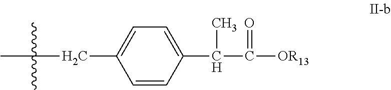
where R₁₃ is selected from the group consisting of the formulas of III-a, III-b, III-c, III-d, and III-e: 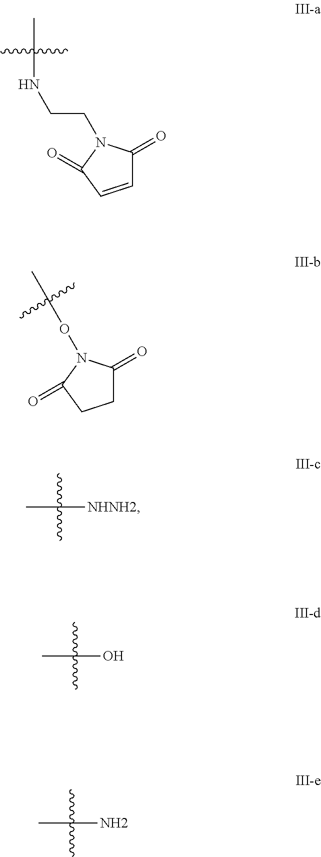
X₂ is the same as X₁, or a group of the formula IV below: 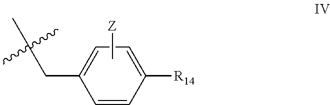
where R₁₄ is an optionally substituted alkyl or optionally substituted phenyl group, and Z is selected from the group consisting of H, SO₃H, optionally substituted alkyl, and optionally substituted phenyl;
wherein the compound is coupled to a species selected from a biomolecule, a synthetic dye, a substrate, a probe, a linker, a target, a low affinity false target, a member of a binding pair, a small molecule, a polymer, an inert surface, a microparticle, a nanoparticle, and/or an optically active species.

51. The conjugate of claim 50 wherein the biomolecule comprises proteins, peptides, polynucleotides, polysaccharides, antibodies, antibody fragments, nucleic acid, triglycerides, lipoproteins, and lectins.

52. A conjugate comprising a compound of the general formula I: 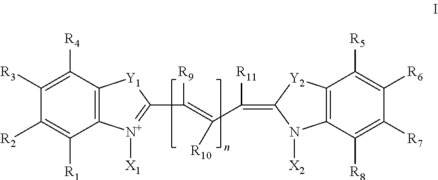
where:
R₁ to R₈ are independently selected from the group consisting of H, SO₃H, optionally substituted alkyl, or optionally substituted heteroalkyl, wherein any two adjacent members of R₁ to R₈ taken together can form an optionally substituted 5-7 membered mono- or poly-unsaturated fused ring optionally containing one or more ring heteroatoms;
R₉, R₁₀, and R₁₁ are independently selected from the group consisting of H, alkyl, alkoxy, heteroalkyl, heteroalkyloxy, —CN, or wherein any two adjacent members of R₉, R₁₀, and R₁₁ may be covalently joined to form an optionally substituted 4-7 membered mono- or poly-unsaturated ring optionally containing one or more ring heteroatoms;
Y₁ and Y₂ are independently selected from the group consisting of O, N, S, and —CR′R″— where R′ and R″ are independently H or C₁-C₁₈ alkyl;
X₁ is a group of the formula II-a: 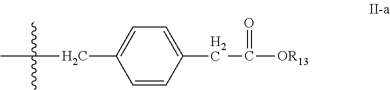
where R₁₃ is selected from the group consisting of the formulas of III-a, III-b, III-c, III-d, and III-e: 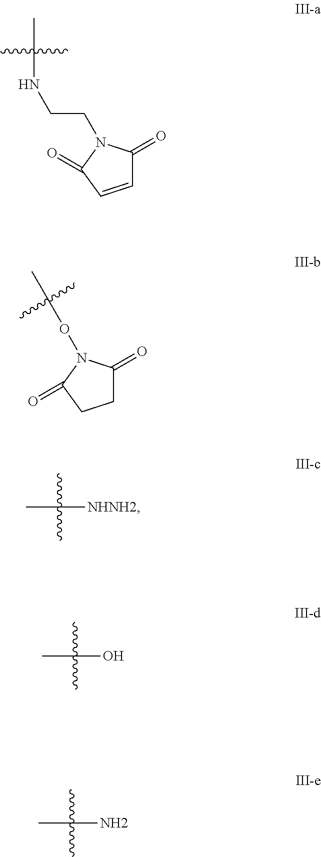
X₂ is the same as X₁, or a group of the formula IV below: 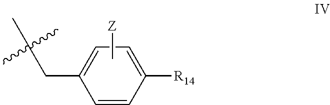
where R₁₄ is an optionally substituted alkyl or optionally substituted phenyl group, and Z is selected from the group consisting of H, SO₃H, optionally substituted alkyl, and optionally substituted phenyl; and
n is zero, 1, 2, or 3,
or an isomer, ester, amide, acid halide, acid anhydride, and/or salt thereof,
provided that when Y₁ and Y₂ are both —C(CH₃)₂—, R₁₃ does not represent III-d or ester thereof;
wherein the compound is coupled to a species selected from a biomolecule, a synthetic dye, a substrate, a probe, a linker, a target, a low affinity false target, a member of a binding pair, a small molecule, a polymer, an inert surface, a microparticle, a nanoparticle, and/or an optically active species.

53. The conjugate of claim 52 wherein the biomolecule comprises proteins, peptides, polynucleotides, polysaccharides, antibodies, antibody fragments, nucleic acid, triglycerides, lipoproteins, and lectins.
